(19) United States
(12) Patent Application Publication
(10) Pub. No.: US 2024/0269318 A1
(43) Pub. Date: Aug. 15, 2024

(54) NANO-DELIVERY SYSTEMS COMPRISING MODIFIED LIPIDS AND USE THEREOF

(71) Applicant: TECHNION RESEARCH & DEVELOPMENT FOUNDATION LIMITED, Haifa (IL)

(72) Inventors: Avi SCHROEDER, Haifa (IL); Mor SELA, Haifa (IL)

(21) Appl. No.: 18/626,620

(22) Filed: Apr. 4, 2024

Related U.S. Application Data
(63) Continuation of application No. PCT/IL2022/051055, filed on Oct. 3, 2022.
(60) Provisional application No. 63/251,739, filed on Oct. 4, 2021.

(51) Int. Cl. A61K 47/69; A61P 25/16; B82Y 5/00

(52) U.S. Cl. CPC A61K 47/6911; A61P 25/16; B82Y 5/00

(57) ABSTRACT

In one aspect of the invention, there is provided a nanoparticle comprising a core and a shell, wherein the core comprises a bioactive compound, and the shell comprises a lipid layer comprising a first modified lipid and a additional lipid, wherein the first modified lipid is bound to a targeted moiety via a spacer, and wherein the additional lipid comprises a polymer-bound lipid. The present invention further provides pharmaceutical compositions and methods for therapeutic and/or diagnostic use.

### CROSS-REFERENCE TO RELATED APPLICATIONS

This application is a Bypass Continuation of a PCT International Application No. PCT/IL2022/051055, having International filing date of Oct. 3, 2022, which claims the benefit of priority of U.S. Patent Application No. 63/251,739, filed Oct. 4, 2021, the contents of which are incorporated by reference in their entirety.

### REFERENCE TO AN ELECTRONIC SEQUENCE LISTING

The contents of the electronic sequence listing (TECH-P-0261-PCT.xml; Size: 4,409 bytes; and Date of Creation: Apr. 4, 2024) is herein incorporated by reference in its entirety.

### FIELD OF INVENTION

The present invention is in the field of nano-delivery systems comprising chemically modified lipids, and use thereof such as for delivery of an active agent to a brain of a subject.

### BACKGROUND OF THE INVENTION

The blood-brain barrier (BBB) is the main obstacle when treating brain disorders. There are different types of brain disorders: brain cancer, Epilepsy, other seizure disorders, mental disorders, stroke and Transient Ischemic Attack (TIA), and central nervous system (CNS) diseases. The BBB comprises endothelial cells forming tight junctions and separating the blood from the brain's extracellular fluid. The permeability of the BBB is selective for only small molecules such as nutrients and water. Therefore, the barrier prevents the crossing of therapeutic drugs required for treating brain disorders.

Neurodegenerative diseases, the fourth leading cause of death in the developed world after heart diseases, cancer, and stroke, affect millions worldwide, but it is more common among the aging population. Neurodegenerative diseases share common features such as cerebral deposits of misfolded protein aggregates and extensive neuronal loss and synaptic abnormalities. People who suffer from these debilitating disorders lose their skilled movements, feelings, cognitive, and memory abilities.

Today, no treatment can cure neurodegenerative diseases, and patients are confined to symptoms-reducing therapies, such as medications and surgery, to reduce complications and function as normally as possible. Thereby, the need for new therapeutic approaches for brain disorders and neurodegenerative diseases and the limitations caused by the BBB are advancing the use of nanotechnology to establish targeted drug delivery to the CNS. Nanoparticles can be highly suitable drug carriers to the brain owing to their physical and chemical properties.

However, the most effective nanoparticles developed for the CNS target site still accumulate significantly in other body regions, such as the liver and kidney. Therefore, there is a need for developing drug delivery systems that can efficiently overcome the BBB and target specific neuron cells and so minimize the off-target effect and might enhance the potential therapeutic value.

### SUMMARY OF THE INVENTION

According to one aspect, there is provided a conjugate comprising a lipid covalently bound to a targeting moiety having a binding affinity to a CNS receptor, wherein: the targeting moiety is bound to the lipid via a spacer; and the targeting moiety has a molecular weight (MW) of less than 1000 Da.

In one embodiment, the targeting moiety is selected from the group consisting of Cotinine, GABA, Caffeine, Aspartic acid, Ritalinic acid, Ketamine, Serotonin, Memantine and Cocaine, or any derivative, any isomer or metabolites thereof and any combination thereof.

In one embodiment, the spacer comprises a biocompatible polymer.

In one embodiment, the biocompatible polymer is PEG.

In one embodiment, the spacer has a molecular weight (MW) between 1000 and 5000 Dalton (Da).

In one embodiment, the conjugate comprises the targeting moiety bound to the spacer via an amide bond.

In one embodiment, a polar group of the lipid comprises a primary amine.

In one embodiment, the lipid comprises a phosphatidyl ethanolamine.

In another aspect, there is provided a nanoparticle comprising a core and a shell; the shell comprises a lipid layer, wherein the lipid layer comprises a phospholipid, a first modified lipid, a second modified lipid and a sterol; the core comprises a bioactive molecule, wherein: each of the first modified lipid and the second modified lipid independently comprises a polymer covalently bound to a lipid; the first modified lipid is covalently bound to a targeting moiety having a binding affinity to a CNS receptor; and a size of the nanoparticle is in a range between 80 and 150 nm.

In one embodiment, the polymer comprises a biocompatible polymer.

In one embodiment, the polymer is a polyether (e.g. PEG).

In one embodiment, the targeting moiety comprises any one of: (i) a protein selected from Lactoferrin, Transferrin, and Insulin, or a combination thereof; and (ii) a small molecule selected form Cotinine, GABA, Caffeine, Aspartic acid, Ritalinic acid, Ketamine, Serotonin, Memantine and Cocaine, or a combination of thereof.

In one embodiment, MW of the polymer of the first modified lipid is between 1000 and 5000 Da; and MW of the polymer of the second modified lipid is between 300 and 1000 Da.

In one embodiment, a molar ratio between the sterol and the phospholipid is between 1:1 and 1:10.

In one embodiment, a MW ratio between the polymer of the first modified lipid and the polymer of the second modified lipid is about 2:1.

In one embodiment, a concentration of the first modified lipid within the nanoparticle is between 0.5 and 10% mol.

In one embodiment, a molar ratio between the first modified lipid and the second modified lipid is between 2:1 and 1:2.

In one embodiment, the phospholipid is characterized by a Tm of less than about 45° C.

In one embodiment, the nanoparticle is a liposome.

In one embodiment, the first modified lipid is the conjugate of the invention.

In another aspect, there is provided a pharmaceutical composition, comprising the nanoparticle of the invention and a pharmaceutically acceptable carrier.

In one embodiment, the pharmaceutical composition is formulated for systemic or local administration.

In one embodiment, the pharmaceutical composition is for use in the prevention or treatment of a disease or disorder in a subject in need thereof.

In one embodiment, the disease or the disorder comprises a neurodegenerative disorder, a neuroinflammatory disorder, a proliferative disease, brain cancer, epilepsy and other seizure disorders, mental disorders, stroke and Transient Ischemic Attack (TIA) and central nervous system (CNS) diseases, or any combination thereof.

In another aspect, there is provided a method of preventing or treating a disease or disorder in said subject, the method comprising administering to said subject a therapeutically effective amount of the pharmaceutical composition of the invention.

In one embodiment, the disease or disorder is a brain disease or disorder.

In one embodiment, the disease or the disorder comprises a neurodegenerative disorder, a neuroinflammatory disorder, a proliferative disease, brain cancer, epilepsy and other seizure disorders, mental disorders, stroke and Transient Ischemic Attack (TIA) and central nervous system (CNS) diseases, or any combination thereof.

Further embodiments and the full scope of applicability of the present invention will become apparent from the detailed description given hereinafter. However, it should be understood that the detailed description and specific examples, while indicating preferred embodiments of the invention, are given by way of illustration only since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-C: Targeted small molecules nanoparticles fabrication and testing targeted nanoparticles uptake in vitro. A. Liposomes were constructed of 64.4% DPPC, 30% cholesterol, 2.5% DSPE-PEG-1000-Moiety, 2.5% DSPE-PEG-550 and 0.6% DSPE-Cy5 at 50 mM total lipid concentration. B. Data table of average diameter (nm), PDI and particle concentration of different liposomal formulations. C. hCMEC/d3 cells were incubated with media supplemented with liposomes labeled with Cy5 for 30 minutes, and then the cells were washed and analyzed using flow cytometry. Results are shown as mean±SEM. The statistical analysis was done using One-way ANOVA (n=4) ****p<0.0001.

FIGS. 2A-B: Biodistribution of targeted lipid nanoparticles in vivo. Targeted liposomes and control liposomes labeled with Cy5 were intravenously injected into healthy mice, and after 2 hr., a perfusion and brains extraction were done, A. The brains were imaged B., and the liposomes' accumulation was quantified using IVIS ex-vivo imaging. Results are shown as mean±SEM. The statistical analysis was done using a T-test (n=3) *P=0.0156.

FIGS. 3A-D: Targeting protein-nanoparticles fabrication. A. A scheme of the fabrication process of protein-targeted liposomes; the liposomes are constructed of DPPC (64.4%), cholesterol (30%), DSPE-PEG-2000-Protein moiety (2.5%), DSPE-PEG-1000-Methyl (2.5%), and DSPE-Cy5 (0.6%) at final 50 mM total lipid concentration. B. Average diameter, PDI, zeta potential, and particles concentration data table of the liposomes. C. Quantification of protein moiety units on the liposome surface using BCA analysis. D. Cryo-TEM imaging 1(I) gold nanoparticles (GNP)-Transferrin (TF) liposomes; (II) GNP with untargeted liposomes (negative control). (2) By Fiji imaging software analysis, a scatterplot of GNP-liposomes was created. Results are shown as mean±SEM.

FIGS. 4A-G: SynO4 antibody activity. A. A schematic illustration of a direct ELISA assay. B. Calibration curve of known SynO4 antibody concentrations. C. IC50 curve of SynO4 antibody inhibition by AS aggregates. D. SynO4 antibody was pre-treated at different temperatures (35, 45, 55, and 65° C.) for 1 hour and immediately measured by ELISA to create an IC50 curve. E. Maximal activity of SynO4 antibody F. SynO4 antibody pre-treated at different temperatures (35, 45, 55, and 65° C.) for 1 hour, stored overnight at 4° C., and the day after measured by ELISA to create IC50 curve. G. Maximal activity of SynO4 antibody after an overnight at 4° C. Results are shown as mean±SEM. The statistical analysis was done using One-Way ANOVA. N=3, ****p<0.0001.

FIGS. 5A-E: Characterization of TF targeted lipid nanoparticles loaded with SynO4 mAb. A schematic representation of TF-SynO4-liposomes fabrication. B. Quantifying of TF moiety units on the liposome surface using BCA analysis C. Quantifying SynO4 conc. in TF-liposomes by ELISA assay. D. Average diameter, PDI, and zeta potential data table of targeted and untargeted liposomes. E. In vitro drug release profile of TF-SynO4-liposomes compared to free SynO4 mAb in PBS over 48 hr. at 37° C. Results are shown as mean±SEM. The statistical analysis was done using T-test, n>3.

FIGS. 6A-D: In vitro BBB endothelial cellular uptake of protein targeted nanoparticles. A. A schematic representation of two different liposome formulations; I A liposome composes of the main lipid (DPPC), cholesterol, Cy5-DSPE, and PEG-lipids—half a long PEG-lipid-Transferrin and half a long PEG-lipid-Methyl; II A liposome composes of the main lipid (DPPC), cholesterol, Cy5-DSPE and PEG-lipids—half a long PEG-lipid-Transferrin and half a short PEG-lipid-Methyl. B. FACS analysis uptake of different liposomal formulations in brain endothelial cells (hCMEC/D3); there is a better uptake by the formulation that consists of a long PEG linker and a short PEG lipid. C. FACS analysis uptake of Insulin liposome, Transferrin liposome, and Lactoferrin liposome compared to control liposome without targeting; there is more uptake when the liposome consists of a protein targeted lipid. D. A scan of various molar ratios of liposome: protein moiety was done, and then the uptake of the different formulations was tested in hCMEC/D3 cells by FACS analysis. Results are shown as mean±SEM. The statistical analysis was done using One-Way ANOVA. N=3, *p=0.0365 **p=0.0032 ***p=0.0002 ****<p0.0001.

FIGS. 7A-B: Uptake of TF-liposomes in hCMEC/D3 cells and crossing of TF-liposomes in cell-based in vitro BBB model. A. Super-resolution images showing cellular uptake of TF liposomes in brain endothelial cells (hCMEC/D3). The liposomes were labeled by Cy5 dye (red), the transferrin receptors, which are expressed by the cells, were stained by an anti-Trf antibody (green) and the cell nucleus was stained by Hoechst dye (blue). B(i). A schematic representation of the BBB model development. A co-culture cell model containing BMECs and neuron cells; BMECs seeded on the apical side and neurons seeded on the plate bottom. The outside medium was taken for fluorescent measurements and for Cryo-TEM imaging. B(ii). Cy5-TF-lipo gradually crossed the designed in vitro BBB model over a period of 24 hr. B(iii). The Cryo-TEM image shows that the liposomes retain their shape after the crossing. Results are shown as mean±SEM.

FIGS. 8A-B: TF-SynO4 lipo uptake in neuron cells. A(i). A schematic representation of PD-SH-SY5Y cells. AS aggregates were seeded with differentiated SH-SY5Y cells and after 6 hr., the cells were washed and incubated by free SynO4 mAb and TF-SynO4 lipo overnight. A(ii). Super-resolution images showing the cellular uptake of the TF-SynO4-liposomes compared to free SynO4 mAb. The merged image shows a colocalization (yellow) between the mAb (red) and AS aggregates (green) after the treatment of liposomes. A(iii). The graph shows that when the SynO4 is delivered by liposomes, the detection between it and the aggregates is above 80% compared to the free antibody. A(iv). About ˜25% of antibody is released from TF-liposomes in SH-SY5Y cell culture overnight. B(i). A schematic representation of PD-primary cortical neurons. The cells were infected by the AAV virus to express mutant AS aggregates and after 3 days, they were washed and incubated with TF-SynO4 liposomes and free Syn-O4. B(ii). Confocal image showing the expression of AS aggregates by GFP (yellow), the cellular uptake of TF-lipo (pink,) and the SynO4 antibody (green); there was no fluorescent signal when the cells were treated by free mAb. B(iii). The imaging statistical analysis shows that when the antibody is administered alone, it penetrates the cells much less efficiently than when it is delivered by liposomes. Results are shown as mean=SEM. The statistical analysis was done using One-Way ANOVA. n>9, *p=0.0346, ****p<0.0001.

FIGS. 9A-B: Therapeutic efficacy of TF-SynO4 liposomes in neuron cells. A(i). A schematic representation of apoptosis assay in PD SH-SY5Y cells. The cells were infected by the AAV virus to express mutant AS aggregates and after 24 hr., they were washed and incubated by different treatments overnight; the following day an apoptosis kit was used, and the apoptosis/necrotic level was measured by FACS analysis. A(ii) The graph shows that transferrin TF-SynO4 liposomes treatment results in a significant reduction of apoptotic cells level compared to the other treatments, meaning that there is a potential therapeutic efficiency of the liposomes delivery system. B(i) dSTORM imaging of primary neurons, that were infected by the AAV virus to express mutant AS aggregates and incubated by TF-SynO4-liposomes and free SynO4. In the left, images of Cy5-TF-liposomes uptake (red) and AS aggregates are labeled by anti-AS antibody (green); in the right, images of Cy3-Syn-O4 uptake (green) and AS aggregates are labeled by anti-AS antibody (red). B(ii). dSTORM statistical graph analysis indicates that TF-SynO4 liposomes significantly reduce AS aggregates in PD-induced cells in comparison to free SynO4 treatment. Results are shown as mean±SEM. The statistical analysis was done using One-Way ANOVA. n>3, ***p=0.0003, ****p<0.0001.

FIGS. 10A-F: Viral PD model establishment. A. Frozen sections (SN and striatum) of healthy mice brains stained with anti-TH Ab (red) and DAPI (blue). Scale bar 1 mM. B. Frozen sections (SN) of PD injected mouse (to the right hemisphere) 2 weeks after injection stained with anti-AS (green) and DAPI (blue). Two zoom-in images to the right show aggregation of the AS protein and localization inside and around neurons. Scale bar 1 mM. C. Frozen sections (SN) of PD injected mouse (to the right hemisphere) 4 weeks after injection stained with anti-AS (green) and anti-TH (red). Scale bar 1 mM. D. Same image as in C showing only the anti-TH staining. The white rectangle indicated the SN compartment where the staining in the left (not injected part) shows more TH-positive neurons compared to the right side. Scale bar 1 mM. E. Zoom-in image of the AS expression of image C (scale bar 415 um); aggregates and fibrils are noticeable. F. Western blot analysis of brain protein extract (in different conditions mentioned below the image) of the right and the left hemispheres separately, 2 weeks after virus injection. The AS protein was stained using anti-AS Ab. There is strong staining of the AS protein at 17 kDa in the right brain extract and almost no expression in the left.

FIGS. 11A-C: SynO4 detection in PD-induced brain. A. Fluorescent histology images of the substantia nigra 24 hours after intravenous injection of 3 different treatments: 3 mg/kg syn-O4-liposomes, 7.5 mg/kg of free syn04 antibody, and no treatment group (control). The syn-O4 antibody is labeled with Cy5 (red), liposomes are labeled with Cy3 (green) and nuclei are stained with DAPI (blue). Co-localization of Cy5-syn-O4 and Cy3-liposomes is shown as a yellow signal. 2×2 tile images of ×63 magnification. B. Zoomed-in images of the top panel. ×63 magnification. C Whole PD-induced brain Elisa quantification of IgG1 (SynO4 isotype). Results are shown as mean±SEM. The statistical analysis was done using One-Way ANOVA. n=5, *p=0.04.

FIGS. 12A-D: Liposomes brain distribution. A. RT-PCR analysis of Trf1 receptor. Transferrin liposomes and untargeted liposomes labeled with Cy5 were intravenously injected into PD mice (8 weeks post injection) and into healthy mice; after 12 hr., a perfusion and brains extraction were done. B. Ex-vivo imaging of the brains. C. Liposomes' accumulation was quantified using IVIS ex-vivo imaging software. TF-lipo labeled with Cy5 were intravenously injected into PD mice (8 weeks post injection) and into healthy mice. After 12 hr. the brains were perfused, extracted, and dissociated into single cells. D. FACS analysis results of liposomes percentage amount in PD brains compared to health brains. E. Representative pie charts of brain cell types in PD brain (i) and in healthy brain (ii); each pie chart shows the Cy5 positive percentage and the Cy5 negative percentage. Results are shown as mean±SEM. The statistical analysis was done using One-Way ANOVA (n=5), *p=0.0285 **p=0.00067 ***p=0.0067 ****p<0.0001.

FIGS. 13A-B: Liposomes biodistribution. A. Transferrin liposomes and untargeted liposomes labeled with Cy5 were intravenously injected into PD mice (8 weeks post injection) and into healthy mice; after 12 hr., a perfusion and organs extraction were done. B. Ex-vivo imaging of the brains. C. liposomes' accumulation was quantified using IVIS ex-vivo imaging software. Results are shown as mean±SEM. The statistical analysis was done using One-Way ANOVA (n=5), **p=0.0054.

FIGS. 14A-G: SynO4 delivered by transferrin targeted liposomes capacity alpha synuclein aggregation in mouse Parkinson model. A. Healthy mice received unilateral AAV injection encoding human alpha synuclein. Mice were after injected every other day with the different treatments, free SynO4 (free Ab) and transferrin liposomes loaded with SynO4 (liposomes) for period 2 or 4 weeks. The brains were harvested after the treatment finished and compared with untreated group (PD) and a non-viral injected group (healthy). B. Brain sections of the different groups (healthy, PD, free Ab, liposomes) after 2 weeks of treatment. Sections were stained against aggregated alpha synuclein and dopaminergic neurons. C. Brain sections of the different groups (healthy, PD, free Ab, liposomes) after 4 weeks of treatment. Sections were stained against aggregated alpha synuclein and dopaminergic neurons. D. Quantification of the aggregates alpha synuclein in different brain sections after 2 weeks of treatment. E. Quantification of extra cytoplasmatic aggregates reduction (%) after 2 weeks of treatment. F. Quantification of the aggregated alpha synuclein in different brain sections after 4 weeks of treatment. G. Quantification of extra cytoplasmatic aggregates reduction (%) after 4 weeks of treatment. Results are shown as mean±SEM. One-Way ANOVA was used for statistical analysis. * p<0.05, **p<0.01, ***p<0.001, ****p<0.0001.

### DETAILED DESCRIPTION OF THE INVENTION

The present invention, in some embodiments thereof, provides a conjugate comprising a lipid, metabolite, or a derivative covalently bound to a targeting moiety via a spacer, wherein the targeting moiety has a binding affinity to a CNS receptor. In some embodiments, the targeting moiety comprises a small molecule characterized by molecular weight (MW) of less than 1000 Da capable of reversibly binding to a CNS receptor.

In another aspect of the invention, there is provided a nanoparticle comprising a core and a shell, wherein the core comprises a bioactive molecule; and the shell comprises a lipid layer, wherein the lipid layer comprises a phospholipid, a first modified lipid, a additional lipid, and a sterol. In some embodiments, the first modified lipid is or comprises the conjugate of the invention. The present invention further provides pharmaceutical compositions and therapeutic and/or diagnostic methods.

According to some embodiments, the invention is based, in part, on the surprising findings that liposomes comprising inter alia modified phospholipids conjugated to a targeting moiety (a small molecule, such as Memantine, or to a protein, such as Lactoferrin) can overcome the restrictive mechanisms of the blood-brain barrier as well as provide a nano-delivery system for targeted delivery of biologically active agents into the brain.

### Conjugates

In one aspect of the invention, there is provided a conjugate comprising a lipid and/or a derivative, or a metabolite thereof, covalently bound to a targeting moiety having a binding affinity to a CNS receptor. In some embodiments, the targeting moiety is covalently bound to the lipid or to a metabolite and/or to a derivative thereof via a spacer. In some embodiments, each lipid is bound to one or more targeting moieties, wherein the targeting moieties are the same or different.

In some embodiments, the lipid comprises a hydrophobic (e.g. hydrocarbon) tail and a polar group. In some embodiments, the polar group is hydrophilic (e.g. comprising a heteroatom). In some embodiments, the polar group is positively charged in an aqueous solution at a pH below 9, or below 8. In some embodiments, the lipid is an ionizable lipid, comprising an ionizable polar group. In some embodiments, the ionizable polar group is capable of undergoing protonation in an aqueous solution at a pH below 9, or below 8. In some embodiments, the ionizable polar group comprises an amine (e.g. a primary, a secondary, a tertiary amine and/or a heterocyclic amine).

In some embodiments, the lipid is or comprises one or more phospholipids. In some embodiments, the phospholipid is a liposome-forming lipid. As used herein, the term “liposome forming lipid” encompasses phospholipids which, upon dispersion or dissolution thereof in an aqueous solution at a temperature above a transition temperature (Tₘ), undergo self-assembly so as to form stable liposomes. As used herein, the term Tm refers to a temperature at which phospholipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the phospholipid is or comprises phosphatidyl ethanolamine (PE). In some embodiments, the phospholipid is or comprises distearyl phosphatidylethanolamine (DSPE). Other phospholipids are well-known in the art; some of them are described herein below.

In some embodiments, the spacer of the invention is or comprises a linear or a branched chain. In some embodiments, the spacer of the invention is or comprises a backbone comprising a linear or a branched chain.

In some embodiments, the linker of the invention comprises a biocompatible polymer. In some embodiments, the biocompatible polymer is at least partially biodegradable.

In some embodiments, the spacer comprises a polymer. In some embodiments, the spacer is covalently bound to the lipid. In some embodiments, the backbone (e.g., a polymer chain) is covalently bound to the lipid and to the targeting moiety. In some embodiments, the backbone of the spacer comprises a first end covalently bound to the lipid and a second end covalently bound to the targeting moiety.

In some embodiments, the polymer is a biocompatible polymer. In some embodiments, the biocompatible polymer is a biodegradable polymer. In some embodiments, the biocompatible polymer comprises polyglycol ether, a polyester, a polyamide or any combination or a co-polymer thereof.

In some embodiments, the biocompatible polymer is selected from the group consisting of a polyether, a polyacrylate or an ester thereof, a polyacrylamide, a polyester (e.g. polylactide, polyglycolate), a polyanhydride, a polyvinyl alcohol, a polysaccharide, a poly(N-vinylpyrrolidone), a polyoxazoline, a poly(amino acid), or any salt, any combination, or a co-polymer thereof. Each possibility represents a separate embodiment of the invention.

As used herein, the terms “peptide”, “polypeptide”, “polyamino acid” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide”, “polyamino acid” and “protein” apply to naturally occurring amino acid polymers including or consisting essentially of 21 naturally occurring amino acids. In one embodiment, the terms “peptide”, “polypeptide”, “polyamino acid” and “protein” apply to naturally occurring amino acid polymers including or consisting essentially of 21 naturally occurring amino acid residues bound to each other via alpha peptide bonds. In one embodiment, the terms “peptide”, “polypeptide”, “polyamino acid” and “protein” apply to naturally occurring amino acid polymers including or consisting essentially of 21 naturally occurring amino acid residues bound to each other via a primary amide bond. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between alpha amino group of the first amino acid and alpha carboxy group of the next following amino acid. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between (i) alpha amino group, or alpha carboxy group of the first amino acid, and (ii) a side chain amino group, or a side chain carboxy group of the next following amino acid. In one embodiment, the terms “peptide”, “polypeptide”, “polyamino acid” and “protein” apply to naturally occurring amino acid polymers including or consisting essentially of 21 naturally occurring amino acids. In another embodiment, the terms “peptide”, “polypeptide”, “polyamino acid” and “protein” apply to amino acid polymers in which one or more amino acid residue is an artificial chemical analogue of a corresponding naturally occurring amino acid.

The term “artificial chemical analogue” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In some embodiments, the biocompatible polymer is a hydrophilic polymer. In some embodiments, the biocompatible polymer is or comprises a polyether.

In some embodiments, the polyether comprises a backbone comprising a plurality of alkoxylate-based repeating units. In some embodiments, the polyether is represented by a general formula: —(RO)ₓ—, wherein R represents C1-C10 alkyl; and x is an integer ranging between 2 and 1000. In some embodiments, R represents an alkyl comprising between 1 and 10, between 1 and 2, between 2 and 4, between 4 and 10, between 2 and 5, between 1 and 5, between 5 and 10 carbon atoms, including any range between. In some embodiments, x is between 2 and 1000, between 2 and 100, between 2 and 10, between 2 and 50, between 50 and 100, between 2 and 200, between 100 and 200, between 200 and 500, between 500 and 1000, including any range between.

In some embodiments, the polyether is or comprises polyethylene glycol (PEG) or a derivative thereof.

Where appropriate, the abbreviation (PEG) is used in combination with a numeric suffix which indicates the average molecular weight of the PEG. A form of PEG or a PEG species is a PEG or PEG derivative with a specified average molecular weight.

As used herein, “PEG or derivatives thereof” refers to any compound including at least one polyethylene glycol moiety. PEGs exist in linear forms and branched forms comprising a multi-arm and/or grafted polyethylene glycols. A PEG derivative may further comprise a functional group. A PEG derivative may be mono-, di-, or multifunctional polyethylene glycol.

Exemplary functional groups include, but are not limited to, the following: a hydroxyl, a carboxyl, a thiol, an amine, a phosphate, a phosphonate, a sulfate, a sulfite, a sulfonate, a sulfoxide, a sulfone, an amide, an ester, a ketone, an aldehyde, a cyano, an alkyne, an azide, and an alkene, or a combination thereof.

In some embodiments, the polymer (and/or the spacer) has an MW between 800 to 5,000 Da, including any range between.

In some embodiments, the polymer (and/or the spacer) has an MW between 800 and 2,000 Da, between 800 and 1,000 Da, between 800 and 1,500 Da, between 800 and 900 Da, between 900 and 1,000 Da, between 1,000 and 1,100 Da, between 1000 and 1,200 Da, between 800 and 1,200 Da, between 1,000 and 3,000 Da, between 1,000 and 5,000 Da, between 1,000 and 7,000 Da, between 1,000 and 10,000 Da, between 2,000 and 3,000 Da, between 2,000 and 5,000 Da, between 2,000 and 7,000 Da, between 2,000 and 10,000 Da, between 3,000 and 5,000 Da, between 3,000 and 7,000 Da, between 3,000 and 10,000 Da, between 5,000 and 7,000 Da, between 5,000 and 10,000 Da, between 7,000 and 10,000 Da including any range between. Each possibility represents a separate embodiment.

According to some embodiments, the polymer (and/or the spacer) has an MW of at least 800 Da, at least 900 Da, at least 800 Da, at least 1,000 Da, at least 1,200 Da, at least 1,500 Da, at least 2,000 Da, including any range between. Each possibility represents a separate embodiment. According to some embodiments, the polymer (and/or the spacer) has an MW of at most 2,000 Da, at most 3,000 Da, at most 4,000 Da, at most 5,000 Da, and at most 7,000 Da. Each possibility represents a separate embodiment.

In some embodiments, the spacer of the invention further comprises a linker (e.g., an amide bond, an ester bond, a click reaction product, a thioester bond, a disulfide bond, a natural and/or unnatural amino acid, alkyl, a urea bond, including any derivative or a combination thereof). In some embodiments, the first end and/or the second end of the spacer each independently comprises one or more linkers.

In some embodiments, the first end is covalently bound to the lipid via a linker; and the second end is covalently bound to the targeting moiety via a linker, wherein each linker is as described herein. In some embodiments, the linker is or comprises a click reaction product (e.g., a covalent linkage such as a cyclization reaction product and/or a succinimide-thioether moiety formed via a click reaction).

Click reactions are well-known in the art and comprise inter alia Michael addition of maleimide and thiol (resulting in the formation of a succinimide-thioether); azide-alkyne cycloaddition; Diels-Alder reaction (e.g., direct and/or inverse electron demand Diels Alder); dibenzyl cyclooctyne 1,3-nitrone (or azide) cycloaddition; alkene tetrazole photo click reaction, etc.

In some embodiments, the conjugate of the invention is represented by Formula 1:

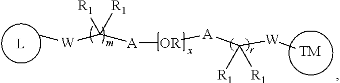

wherein L represents the lipid of the invention; TM represents the targeting moiety of the invention; each r and m independently represents an integer ranging from 0 to 10, including any range between; and x represents an integer ranging between 2 and 1000, including any range between; R represents C1-C10 alkyl; each R1 independently represents a substituent or H; each W independently represents one or more linkers, wherein each linker independently comprises a carbonyl derivative (e.g., —C(═O)NH—, —C(═O)O—, —C(═O)—, —C(═O)S—, —C(═NH)NH—, —C(═NH)O—, —C(═NH)S—), a disulfide bond, or a click reaction product; and each A independently represents a heteroatom (e.g., O, N, NH, or S), or is absent.

In some embodiments, the click reaction product comprises a moiety formed via a click reaction, wherein the click reaction is as described herein above. In some embodiments, the click reaction product comprises a product formed by any of: Michael addition of maleimide and thiol (resulting in the formation of a succinimide-thioether); azide-alkyne cycloaddition; Diels-Alder reaction (e.g., direct and/or inverse electron demand Diels Alder); dibenzyl cyclooctyne 1,3-nitrone (or azide) cycloaddition; alkene tetrazole photo click reaction, or any combination thereof.

In some embodiments, the lipid within the conjugate of the invention comprises a lipid (e.g., PE) and/or a derivative thereof. In some embodiments, a derivative of the lipid (e.g., a PE derivative) encompasses a PE-based conjugate. In some embodiments, a derivative of the lipid (e.g., a PE derivative) refers to a metabolite thereof. In some embodiments, a derivative of the lipid (e.g., a PE derivative) refers to PE covalently bound to the spacer via the amino group. Accordingly, in a non-limiting example, a derivative of PE comprises a deprotonated amine.

In some embodiments, the conjugate of the invention is represented by Formula 2:

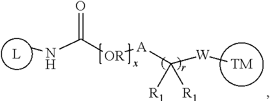

wherein L, TM, r, m, x, R, R1, W, and A are as described herein above. In some embodiments, R is ethyl, and the spacer comprises PEG. In some embodiments, the terminal repeating unit of the polymer (e.g., PEG) is covalently bound to the targeting moiety. In some embodiments, the terminal repeating unit of the polymer is covalently bound to the targeting moiety via an amide bond.

In some embodiments, the conjugate of the invention comprises one or more targeting moieties. In some embodiments, the targeting moiety comprises a molecule capable of binding to one or more central nervous system (CNS) receptor. In some embodiments, binding is a reversible binding. In some embodiments, binding is a non-covalent binding. In some embodiments, the targeting moiety is capable of binding to one or more CNS receptors so as to cross the BBB and undergo internalization into a brain.

In some embodiments, the targeting moiety is or comprises a protein. In some embodiments, the protein has a molecular weight of at least about five kilodaltons (kD). In some embodiments, the protein is selected from insulin, transferrin, a low-density lipoprotein, apolipoprotein A1, B, or E, or lactoferrin, or any combination thereof.

In some embodiments, the targeting moiety is a small molecule, having a MW less than 1,000 Daltons (Da). In some embodiments, the targeting moiety has a MW of between 100 and 1,000 Da, between 100 and 300 Da, between 100 and 500 Da, between 100 and 800 Da, between 300 and 500 Da, between 100 and 1,000 Da, between 500 and 800 Da, between 500 and 1,000 Da, between 800 and 1,000 Da, including any range between. Each possibility represents a separate embodiment.

In some embodiments, the targeting moiety has a MW less than 1,000 Da, less than 900 Da, less than 800 Da, less than 700 Da, less than 600 Da, less than 500 Da, less than 400 Da, less than 300 Da, less than 200 Da, less than 100 Da. Each possibility represents a separate embodiment. In some embodiments, the targeting moiety has a MW of more than 100 Da, more than 200 Da, more than 300 Da, more than 400 Da, more than 500 Da, more than 600 Da, more than 700 Da, more than 800 Da, or more than 900 Da. Each possibility represents a separate embodiment.

In some embodiments, the targeting moiety is a ligand having a binding affinity to the receptor of interest (e.g. a cell surface receptor, such as a CNS receptor). In some embodiments, the ligand is a polyamino acid. In some embodiments, the ligand is or comprises a small molecule recognizable by the target (e.g. cell receptor). In some embodiments, the ligand is or comprises a natural ligand of a cell receptor. In some embodiments, the ligand is or comprises a natural ligand of a cell surface receptor. In some embodiments, the natural ligand is a small molecule (e.g. a natural compound). In some embodiments, the ligand binds a target on the cell membrane. In some embodiments, the ligand binds an extracellular target on the cell membrane. In some embodiments, the ligand comprises a single specie or a plurality of chemically distinct species.

In some embodiments, the ligand hybridizes to its target. In some embodiments, the ligand is complementary to its target. In some embodiments, the ligand is an antibody or antigen binding fragment thereof. The structure of antibodies is well known and though a skilled artisan may not know to what target an antibody binds merely by its CDR sequences, the general structure of an antibody and its antigen binding region can be recognized by a skilled artisan.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, cither alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the antibody comprises a heavy chain and a light chain. In some embodiments, the antibody is a heavy chain only antibody. In some embodiments, the antibody is an antibody mimetic.

In some embodiments, the targeting moiety comprises a CNS receptor ligand and/or a metabolite and/or a derivative thereof. In some embodiments, the terms “derivative” and “metabolite” (e.g., when referring to the targeting moiety) are used herein interchangeably.

In some embodiments, a derivative of the CNS receptor ligand comprises a chemically modified ligand (e.g., aminated-, carboxylated-, thiolated-, esterified-, amidated derivative, or comprising an amine-, and/or carboxy-protecting group, etc.), a structural isomer, a tautomer, a stereoisomer, or a metabolite of the ligand. In some embodiments, a derivative of the CNS receptor ligand comprises an ester, an amide, and/or a carboxylated derivative of the corresponding ligand. In some embodiments, a derivative substantially maintains the binding affinity to a CNS receptor as compared to the ligand (e.g., a natural ligand of the particular CNS receptor). In some embodiments, a derivative of the CNS receptor ligand substantially maintains the functional properties of the corresponding ligand (e.g., a natural ligand of the particular CNS receptor).

In some embodiments, a CNS receptor is located in a brain of a subject. In some embodiments, a CNS receptor is a transporter capable of internalizing the conjugate into the brain of a subject. In some embodiments, a CNS receptor is selected from: Adenosine receptor, GABA-transporter, Glucose transporter, N-methyl-d-aspartate (NMDA) receptor, α-amino-3-hydroxy-5-methyl-4-isoxazolepropionic acid (AMPA) receptor, Nicotinic cholinergic receptor, ASC transporter, 5-hydroxytryptamine receptor, serotonin receptor, Cannabinoid receptor, Dopamine receptor and norepinephrine transporter, including any tautomer, any stereoisomer (e.g. an enantiomer, or a diastereomer), an ester, an amide, or any combination thereof.

In some embodiments, the targeting moiety comprises any of: Cotinine, GABA, Caffeine, Aspartic acid (D or L), N-Boc D-aspartic acid 1-tert-butyl ester, Ritalinic acid, Ketamine, Serotonin, Memantine, D-glucuronic acid, theophylline-7-acetic acid, trans-4-cotininecarboxylic acid, and Cocaine, including any combination thereof.

In some embodiments, the targeting moiety is devoid of insulin, glucose, glucosamine, transferrin, Mannose-6-phosphate, and folic acid.

Exemplary conjugates of the invention are represented below:

| Compound name | Structure |
|---|---|
| DSPE-PEG1000-Cotinine | 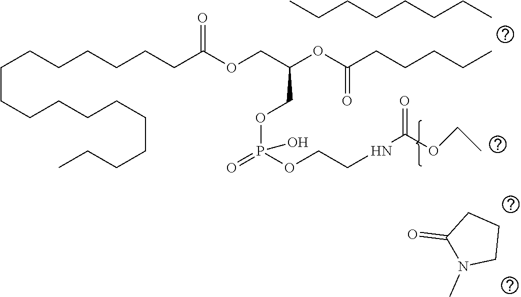 |
| DSPE-PRG1000-γ-Aminobutyric acid (GABA) | 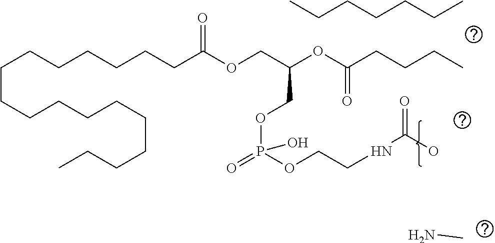 |
| DSPE-PEG1000-Caffeine | 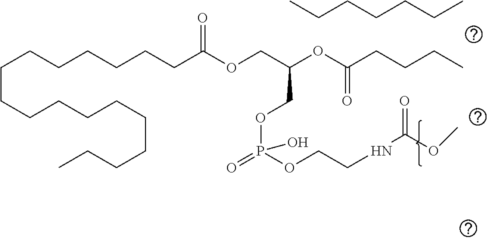 |
| DSPE-PEG1000-D-Aminosuccinic acid (aspartic acid) | 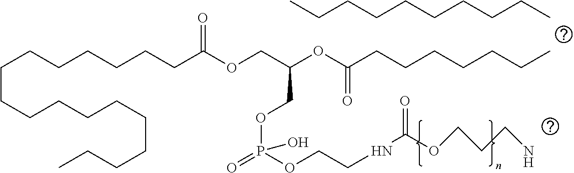 |
| DSPE-PEG1000-Glucose | 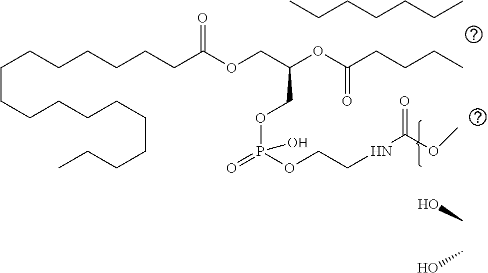 |
| DSPE-PEG1000-GlutarylChloride- Ketamine | 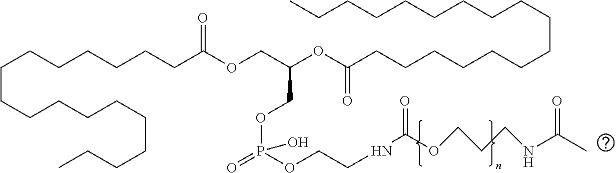 |
| DSPE-PEG1000-GlutarylChloride- Serotonin | 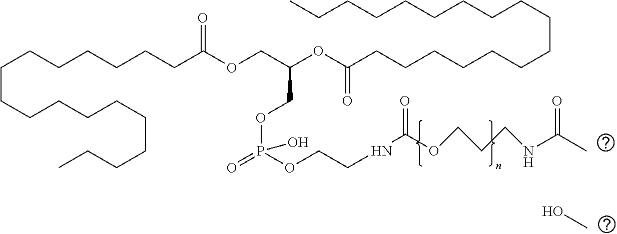 |
| DSPE-PEG1000-GlutarylChloride- Memantine | 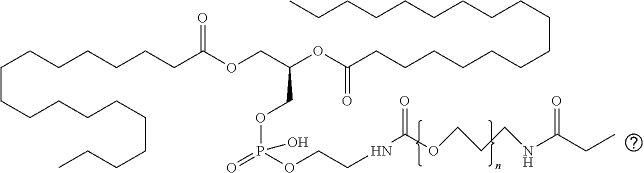 |
| DSPE-PEG1000-Cocaine | 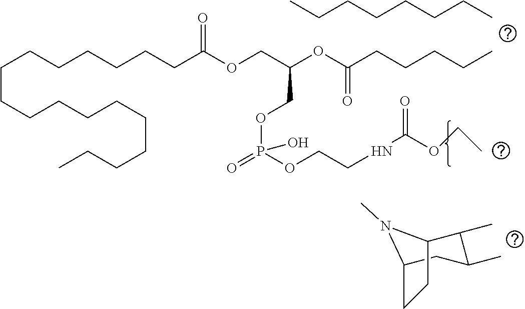 |
| DSPE-PEG1000-Ritalinic acid | 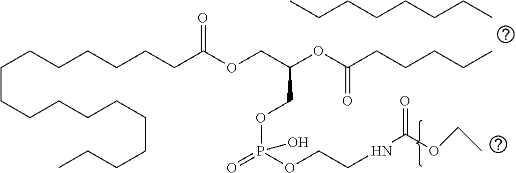 |

*n is an integer ranging between 1 and 100, or between 20 and 50, including any range between, or about 20

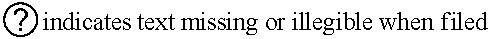

### Nanoparticles

In another aspect. there is provided a carrier for an active agent(s), wherein the carrier is in a form of a core-shell nanoparticle. In some embodiments, the carrier encapsulates the active agent within the core. In some embodiments, the active agent is a small molecule and/or a biologic molecule, such as polypeptide, a polynucleotide, etc. In some embodiments, the shell of the carrier comprises a conjugate of the invention.

In another aspect, there is provided a nanoparticle comprising a core facing or in contact with a shell, wherein: the core comprises a bioactive molecule (the terms bioactive molecule and active agent are used herein interchangeably); and the shell comprises a lipid layer, wherein the lipid layer comprises a phospholipid, a first modified lipid, and an additional lipid; the first modified lipid comprises a lipid-bound to a spacer (e.g., a polymer); and wherein the first modified lipid is covalently bound to a targeting moiety via the spacer, wherein the targeting moiety has a binding affinity to a CNS receptor. In some embodiments, the additional lipid is selected from a helper lipid, a sterol, a PEG-lipid, or any combination thereof.

In another aspect, there is provided a nanoparticle comprising a core facing or in contact with a shell, wherein: the core comprises a bioactive molecule (the terms bioactive molecule and active agent are used herein interchangeably); and the shell comprises a lipid layer, wherein the lipid layer comprises a phospholipid, a first modified lipid, a additional lipid, and a sterol; each of the first modified lipid and the additional lipid independently comprises a lipid-bound to a spacer (e.g., a polymer); and wherein the first modified lipid is covalently bound to a targeting moiety via the spacer, wherein the targeting moiety has a binding affinity to a CNS receptor.

In some embodiments, the first modified lipid is the conjugate of the invention. In some embodiments, the at least one compound of the invention (and optionally the additional lipid), under suitable conditions spontaneously undergo self-assembly in an aqueous solution, so as to form the nanoparticle disclosed herein. In some embodiments, the nanoparticle is a lipid nanoparticle.

In some embodiments, the term “lipid nanoparticle” refers to a nanoparticle (e.g. substantially spherical particle), wherein the shell of the nanoparticle comprises one or more compounds of the invention and optionally one or more lipids (e.g., a helper lipid, such as a cationic lipid, non-cationic lipid; and optionally a sterol, and/or a PEG-modified lipid). Preferably, the lipid nanoparticles are formulated to deliver one or more agents to one or more target cells.

In some embodiments, the nanoparticle has a spherical geometry or shape. In some embodiments, the nanoparticle has an inflated or a deflated shape. In some embodiments, a plurality of core-shell particles is devoid of any characteristic geometry or shape. In some embodiments, the nanoparticle has a spherical shape, a quasi-spherical shape, a quasi-elliptical sphere, a deflated shape, a concave shape, an irregular shape, or any combination thereof.

In some embodiments, the nanoparticles are substantially spherically shaped, wherein substantially is as described herein. In some embodiments, the nanoparticles are substantially elliptically shaped, wherein substantially is as described herein. One skilled in the art will appreciate that the exact shape of each of the nanoparticles may differ from one particle to another. Moreover, the exact shape of the nanoparticle may be derived from any of the geometric forms listed above, so that the shape of the particle does not perfectly fit a specific geometrical form. One skilled in the art will appreciate that the exact shape of the nanoparticle may have substantial deviations (such as at least 5%, at least 10%, at least 20% deviation) from a specific geometrical shape (e.g., a sphere or an ellipse).

In some embodiments, the nanoparticle is characterized by a particle size between 80 and 200 nm, between 80 and 100 nm, between 80 and 130 nm, between 100 and 200 nm, between 100 and 150 nm, between 90 and 150 nm, between 100 and 130 nm, between 130 and 150 nm, between 150 and 200 nm, between 200 and 300 nm, between 80 and 300 nm, between 50 and 300 nm, between 50 and 80 nm, between 80 and 150 nm, including any range between. Each possibility represents a separate embodiment.

In some embodiments, the nanoparticle has a diameter of at least 50 nm, at least 60 nm, at least 70 nm, at least 80 nm, at least 90 nm, and at least 100 nm. Each possibility represents a separate embodiment. In some embodiments, the nanoparticle has a diameter at most 130 nm, at most 150 nm, at most 170 nm, at most 180 nm, at most 200 nm, at most 250 nm, and at most 300 nm. Each possibility represents a separate embodiment.

In some embodiments, the nano-particles of the invention are characterized by a polydispersity index of between 1.03 and 1.3, between 1.03 and 1.05, between 1.05 and 1.1, between 1.1 and 1.15, and between 1.15 and 1.2, between 1.2 and 1.3, including any value and range therebetween. In some embodiments, the nano-particles of the invention are characterized by a median size, as described hereinabove, and are further characterized by polydispersity index of between 1.03 and 1.3, between 1.03 and 1.05, between 1.05 and 1.1, between 1.1 and 1.15, between 1.15 and 1.2, between 1.2 and 1.3, including any value and range therebetween.

In some embodiments, at least 80%, at least 85%, at least 90%, at least 95%, and at least 97% by weight of the nanoparticles of the invention have a particle size in a range of between 80 and 200 nm, between 80 and 100 nm, between 80 and 150 nm, between 80 and 130 nm, between 100 and 150 nm, between 150 and 200 nm, between 200 and 250 nm, between 250 and 300 nm, including any value and range therebetween. In some embodiments, at least 80%, at least 85%, at least 90%, at least 95%, and at least 97% by weight of the nanoparticles of the invention have a particle size in a range of between 80 and 150 nm.

The terms “particle size” and “particle diameter” are used herein interchangeably and refer to an average cross-section size of the nanoparticles (e.g., the largest linear distance between two points on the surface of the nanoparticle) within a liquid composition. In some embodiments, the term “average cross-section size” may refer to either the average of at least, e.g., 70%, 80%, 90%, or 95% of the particles, or in some embodiments, to the median size of the plurality of nanoparticles. In some embodiments, the term “average cross-section size” refers to a number average of the plurality of nanoparticles. In some embodiments, the term “average cross-section size” may refer to an average diameter of substantially spherical nanoparticles.

A person of ordinary skill in the art would be familiar with techniques for measuring particle size, such as dynamic light scattering (DLS).

In some embodiments, the nanoparticle is a lipid nanoparticle. In some embodiments, the nanoparticle is in the form of a vesicle, wherein the vesicle forms a complex/particulate with the carried materials (e.g., biologically active agent) with or without an additional agent such as a polymer, protein, or salt. In some embodiments, the nanoparticle forms a dendrimer-like structure, in which the components of the dendrimer-like structure are conjugated to the polymeric backbone or complexed via Van Der Waals or hydrophobic interactions.

In some embodiments, “vesicle” and “carrier” are synonymous and refer to a particle (e.g., the nanoparticle of the invention) comprising a core and a shell encapsulating or enclosing the core. In some embodiments, the nanoparticle of the invention comprises a core and a shell encapsulating or enclosing the core. In some embodiments, the core is a hollow core or a core filled with a solid or liquid material. In some embodiments, the nanoparticle of the invention may have a spherical or any other geometrical shape. In some embodiments, the nanoparticle of the invention comprises a unilamellar or multilamellar membrane (or lipid layer).

Unstable nanoparticles (e.g., lipid nanoparticles, or LNPs) are disadvantageous for the instantly disclosed invention, wherein unstable refer to nanoparticles that don't retain their shape and/or size and/or decompose within a liquid composition so as to release the biologically active agent therefrom.

In some embodiments, the nanoparticle of the invention is or comprises a lipid-based particle. In some embodiments, the nanoparticle of the invention is or comprises a liposome. In some embodiments, liposomes refer to vesicles with an internal core surrounded by a lipid bilayer/s and are widely used as drug carriers. This is greatly due to their unique characteristics, such as good biocompatibility, low toxicity, lack of immune system activation, and the ability to incorporate both hydrophobic and hydrophilic compounds. As described herein, liposomes are known in the art as artificial vesicles composed of a substantially spherical lipid bilayer which typically, but not exclusively, comprises phospholipids, sterol, e.g., cholesterol, and other lipids.

In some embodiments, the liposomes disclosed herein can be any one or combination of vesicles selected from the group consisting of small unilamellar vesicles (SUV), large unilamellar vesicles (LUV), multilamellar vesicles (MLV), multivesicular vesicles (MVV), large multivesicular vesicles (LMVV, also referred to, at times, by the term giant multivesicular vesicles, “GMV”), oligolamellar vesicles (OLV), and others.

In some embodiments, the liposomes are large unilamellar vesicles (LUV).

In some embodiments, the liposomes are characterized by a proper packing parameter. As used herein and in the art, the packing parameter is a relative measure of given lipid composition and depends on factors such as size relationships between lipid head groups and lipid hydrocarbon chains, charge, and the presence of stabilizers such as cholesterol. It should also be noted that the packing parameter may not be constant. In some embodiments, the parameter is dependent on various conditions which affect the volume of the hydrophobic chain, the cross-sectional area of the hydrophilic head group, and the length of the hydrophobic chain. Factors that can affect these include but are not limited to the properties of the solvent, the solvent temperature, and the ionic strength of the solvent.

In some embodiments, the proper packing parameter is in the range of 0.3 to 1, e.g., 0.3, 0.5, 0.7, 0.9, or 1, including any value and range therebetween.

As used herein, the phrase “lipid nanoparticle” refers to a transfer vehicle, wherein the shell of the carrier comprises one or more lipids (e.g., liposome forming lipids, such as cationic lipids, non-cationic lipids, and PEG-modified lipids) and/or one or more compounds of the invention. Furthermore, the lipid nanoparticles further comprise a non-liposome forming lipid, such as a sterol. Preferably, the lipid nanoparticles are formulated to deliver one or more agents to one or more target cells.

In another embodiment, the carrier is characterized by a negative zeta potential (e.g., measure at a pH between about 6.5 and 7.5). In some embodiments, the carrier is characterized by a negative zeta potential ranging between −0.1 and −30 mV, between −0.5 and −20 mV, between −0.1 and −10 mV, including any range between.

The term “core”, as used herein, refers to the central portion of the particle, with a different composition than the shell. In some embodiments, the core is enclosed by the shell. In some embodiments, the core is bound to the inner portion of the shell.

In some embodiments, the core is a liquid. In some embodiments, the core comprises an aqueous solution. In some embodiments, the core comprises an aqueous solution of the bioactive molecule. In some embodiments, the core comprises a biologically active agent, substantially located therewithin.

In some embodiments, the carrier is stable for a time period ranging between 1 day and 1 year, or more, including any range between. In some embodiments, the term “stable” refers to physical and chemical stability of the carrier (such as being substantially devoid of phase separation, agglomeration, disintegration, and/or substantially retaining the initial loading of the active agent) under appropriate storage conditions. In some embodiments, the term “stable” refers to physical and chemical stability of the carrier within an aqueous solution (e.g., dispersion stability).

In some embodiments, the biologically active molecule comprises one or more distinct bioactive species. In some embodiments, there is a composition comprising a plurality of nano-particles of the invention, wherein the nano-particles are the same or different. In some embodiments, by “different nanoparticles” it is meant to refer to particles that encapsulate different bioactive agents (e.g., drugs). In some embodiments, the biologically active molecule comprises at least one polymer molecule. In some embodiments, the biologically active molecule comprises a small molecule, a macromolecule, an antibody, an oligonucleotide, an antisense RNA, a peptide or any combination thereof. In some embodiments, the bioactive molecule comprises a pharmaceutically active agent (e.g., a drug) and/or a diagnostic agent (e.g., a labeling agent). In some embodiments, a pharmaceutically active agent and/or a diagnostic agent are as disclosed herein below. In some embodiments, the bioactive molecule is attached to and/or encapsulated within the nano-particle (e.g., liposome).

In some embodiments, the shell is or comprises a lipid layer. In some embodiments, the shell is in the form of a membrane. In some embodiments, the shell comprises one or more lipid layers. In some embodiments, the shell comprises a lipid bilayer. In some embodiments, the bioactive agent is bound to the membrane. In some embodiments, the bioactive agent is located between the membrane layers. In some embodiments, the bioactive agent is located within the membrane (e.g., within the lipid bilayer).

The term ‘shell,’ as used herein, refers to the outer portion of the particle, with a different composition than the core. In some embodiments, the shell comprises a targeting moiety.

In some embodiments, the lipid layer comprises a phospholipid (also termed as a “helper lipid). In some embodiments, additional lipid, as used herein, encompasses inter alia a helper lipid. In some embodiments, the phospholipid encompasses a single phospholipid specie or a plurality of chemically distinct phospholipids. In some embodiments, the phospholipid is or comprises a liposome forming lipid, wherein the liposome forming lipid is as described herein above.

In some embodiments, the phospholipids are selected from glycerophospholipids and sphingomyelins. The glycerophospholipids have a glycerol backbone wherein at least one, preferably two, of the hydroxyl groups at the head group is substituted by one or two hydrocarbon tails (chains), typically an acyl, alkyl, or alkenyl tails, and the third hydroxyl group is substituted by a phosphate (phosphatidic acid) or a phospho-ester such as a phosphocholine group (as exemplified in phosphatidylcholine), being the polar head group of the glycerophospholipid or combination of any of the above, and/or derivatives of same and may contain a chemically reactive group (such as an amine, acid, ester, aldehyde or alcohol). The sphingomyelins consist of a ceramide (N-acyl sphingosine) unit having a phosphocholine moiety attached to position 1 as the polar head group. The term “sphingomyelin” or “SPM” as used herein denotes any N-acetyl sphingosine conjugated to a phosphocholine group, the latter forming the polar head group of the sphingomyelin (N-acyl sphingosyl phospholcholines). The acyl chain bound to the primary amino group of the sphingosine (to form the ceramide) may be saturated or unsaturated, branched, or unbranded.

In some embodiments, at least one of the liposome-forming lipid is a phospholipid having one or two C14 to C24 hydrocarbon tails, typically, acyl, alkyl, or alkenyl chain) and have varying degrees of unsaturation, from being fully saturated to being fully, partially, or non-hydrogenated lipids (the level of saturation may affect the rigidity of the liposome thus formed (typically, liposomes formed from lipids with saturated chains are more rigid than liposomes formed from lipids of same chain length in which there are un-saturated chains, especially having cis double bonds).

Further, the lipid membrane may be of natural source (e.g., naturally occurring phospholipids), semi-synthetic or fully synthetic lipid, as well as electrically neutral, negatively, or positively charged.

Examples of liposome forming glycerophospholipids include, without being limited thereto, phosphatidylglycerols (PG) including dimyristoyl phosphatidylglycerol (DMPG); phosphatidylcholine (PC), including egg yolk phosphatidylcholine, soybean PC, sunflower PC, rapeseed PC, krill PC, canola PC, flax seed lecithin, wheat lecithin, dimyristoyl phosphatidylcholine (DMPC, Tm 24° C.), 1-palmitoyl-2-oleoylphosphatidyl choline (POPC), hydrogenated soy phosphatidylcholine (HSPC), distearoylphosphatidylcholine (DSPC, Tm 55° C.); di-lauroyl-sn-glycero-2phosphocholine (DLPC); 1,2-dipalmitoyl-sn-glycero-3-phosphocholine (DPPC, Tm 41° C.); 1,2-dinonadecanoyl-sn-glycero-3-phosphocholine; 1,2-diarachidoyl-sn-glycero-3-phosphocholine (DBPC); 1,2-dihenarachidoyl-sn-glycero-3-phosphocholine; 1,2-dibehenoyl-sn-glycero-3-phosphocholine 1,2-ditricosanoyl-sn-glycero-3-phosphocholine 1,2-dilignoceroyl-sn-glycero-3-phosphocholine; 1-myristoyl-2-stearoyl-sn-glycero-3-phosphocholine; 1-palmitoyl-2-stearoyl-sn-glycero-3-phosphocholine (PSPC); 1-stearoyl-2-palmitoyl-sn-glycero-3-phosphocholine (SPPC); 1,2-di-oleoyl-sn-glycero-3-phosphocholine (DOPC Tm −17° C.); phosphatidic acid (PA), phosphatidylinositol (PI), phosphatidylserine (PS), phosphatidylethanolamine (PE).

The liposome forming lipids or helper lipids may comprise a non-cationic lipid. As used herein, the term “non-cationic lipid” refers to any neutral, or zwitterionic lipid. Non-cationic lipids include, but are not limited to, dioleoylphosphatidylethanolamine (DOPE), distearoylphosphatidylcholine (DSPC), hydrogenated soy phosphatidylcholine (HSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

Further, the liposome forming lipids or helper lipids may be of natural source (e.g., naturally occurring phospholipids), semi-synthetic or fully synthetic lipid, as well as electrically neutral (e.g. zwitterionic), negatively, or positively charged.

Non-limiting examples of neutral phospholipids include but are not limited to diacylphosphatidylcholines, dialkylphosphatidylcholines, sphingomyelins, and diacylphosphatidylethanolamines. Phosphatidylcholines (PC), including those obtained from egg, soybeans or other plant sources or those that are partially or wholly synthetic, or of variable lipid chain length and unsaturation are suitable for use in the present compositions. Synthetic, semisynthetic and natural product phosphatidylcholines including, but not limited to, POPC, DOPC, DMPC, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), and dipalmitoylphosphatidylcholine (DPPC) are suitable phosphatidylcholines for use in the preparation of liposomes. Charged phospholipids can include phosphatidylglycerols, cardiolipins, or headgroup modified lipids such as N-succinyl-phosphatidylethanolamines, N-glutaryl-phosphatidylethanolamines, and PEG-derivatized phosphatidylethanolamines.

Non-limiting examples of cationic lipids include but are not limited to 5-carboxyspermylglycinedioctadecylamide or “DOGS,” N-[1-(2,3-dioleyloxy)propyl]-N,N,N-trimethylammonium chloride or “DOTMA”, 2,3-dioleyloxy-N-[2(spermine-carboxamido)ethyl]-N,N-dimethyl-1-pr-opanaminium or “DOSPA”, 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis, cis-9,12-oc-tadecadienoxy)propane or “CLinDMA”, 2-[5′-(cholest-5-en-3-beta-oxy)-3′-oxapentoxy)-3-dimethyl-1-(cis,cis-9′, 1-2′-octadecadienoxy)propane or “CpLinDMA”, N,N-dimethyl-3,4-dioleyloxybenzylamine or “DMOBA”, 1,2-N,N′-diolcylcarbamyl-3-dimethylaminopropane or “DOcarbDAP”, 2,3-Dilinoleoyloxy-N, N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-di-methylethanamine (DLin-KC2-DMA)).

In some embodiments, the helper lipid is or comprises a non-cationic lipid. As used herein, the term “non-cationic lipid” refers to any neutral, or zwitterionic lipid. Non-cationic lipids include, but are not limited to, dioleoylphosphatidylethanolamine (DOPE), distearoylphosphatidylcholine (DSPC), hydrogenated soy phosphatidylcholine (HSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

In some embodiments, the helper lipid is or comprises DOPE, DSPC, POPE, or any combination thereof.

In some embodiments, the liposome forming lipid is characterized by a Tm less than about 50° C., less than about 47° C., less than about 45° C., less than about 43° C., including any range between.

Non-limiting examples of liposome forming lipids having a Tm of less than 45° C. include, without being limited thereto, POPC (16:0/18:1, Tm=−2° C.), DMPC (14:0, Tm=25° C.) and DPPC (16:0, Tm=41° C.), or a combination thereof.

In some embodiments, the liposome forming lipid has a choline head group.

In some embodiments, the liposome forming lipid comprises one or more saturated and/or unsaturated hydrocarbon tails. In some embodiments, each hydrocarbon tail independently comprises between 14 and 24, between 16 and 24, between 17 and 24, between 17 and 20, between 14 and 17, between 20 and 24, between 18 and 20 carbon atoms, including any range between. In some embodiments, the hydrocarbon tails of the lipid have the same or different chemical composition.

In some embodiments, the liposome forming lipid is a phosphatidylcholine (PC) carrying one or two saturated or unsaturated C14 to C24 hydrocarbon tails. In some embodiments, each of the hydrocarbon tails comprises an unsaturated hydrocarbon. In some embodiments, the liposome forming lipid comprises at least one unsaturated C18 PC. In some embodiments, each of the hydrocarbon tail is independently selected from C18:1, C18:2, and C18:3. In some embodiments, the liposome forming lipid comprises C18:1 and/or C18:2 hydrocarbon tails.

In some embodiments, the liposome forming lipid comprising C18:1 and/or C18:2 hydrocarbon tail is beneficial for the stabilization of bioactive molecules (e.g., one or more antibodies).

In some embodiments, the liposome forming lipid is characterized by a Tm below 45°.

In some embodiments, the additional lipid a non-liposome forming lipid. When referring to a non-liposome forming lipid it is to be understood as referring to a lipid that does not spontaneously form into a vesicle when brought into an aqueous medium (also known as “structural lipid”).

There are various types of lipids that do not spontaneously vesiculate and yet are used or can be incorporated into vesicles. In some embodiments, the non-liposome forming lipid is or comprises a sterol.

In some embodiments, the additional lipid is a sterol. In some embodiments, the lipid layer comprises one or more sterols. Non-limiting examples of sterols include but are not limited to β-sitosterol, β-sitostanol, stigmasterol, stigmastanol, campesterol, campestanol, ergosterol, avenasterol, brassicasterol, fucosterol, cholesterol (CHOL), cholesteryl hemisuccinate, and cholesteryl sulfate, or any combination thereof.

Additional non-limiting examples of structural lipids include but are not limited to: Calcipotriol, campesterol, cholesterol, Daucosterol, DC-cholesterol, Dehydroergosterol, DMAPC-Chol, DMHAPC-Chol, ergosterol, Fucosterol, HAPC-Chol, Lupeol, MHAPC-Chol, OH-C-Chol, OH-Chol, Oleanolic acid, stigmastanol, stigmasterol, Ursolic acid, a hydrophobic vitamin (e.g. Vitamin D2, Vitamin D3, vitamin E, etc.), β-sitosterol, β-Sitosterol-Acetate, β-sitosterol-arginine, β-sitosterol-cysteine, β-sitosterol-glycine, β-sitosterol-histidine, β-sitosterol-serine, or a steroid, including any salt or any combination thereof

In some embodiments, the sterol is a plant-derived sterol, namely, a phytosterol. In accordance with this embodiment, the sterol is selected from the group consisting of β-sitosterol, β-sitostanol, stigmasterol, stigmastanol, campesterol, campestanol, ergosterol, avenasterol, brassicasterol, and any combination thereof.

In some embodiments, a molar concentration of the liposome forming lipid (e.g. of a helper lipid, such as phospholipid) within the shell is between 40 and 95 mol %, between 40 and 50 mol %, between 50 and 70 mol %, between 40 and 60 mol %, between 60 and 70 mol %, between 70 and 80 mol %, between 80 and 95 mol %, including any range between.

In some embodiments, a molar concentration of the liposome forming lipid within the nanoparticle is between 5 and 40 mol %, between 5 and 10 mol %, between 10 and 40 mol %, between 10 and 30 mol %, between 5 and 20 mol %, between 20 and 40 mol %, including any range between.

In some embodiments, a concentration of the non-liposome forming lipid (e.g. sterol) within the nanoparticle is between 20 and 50 mol %, between 20 and 25 mol %, between 25 and 30 mol %, between 30 and 35 mol %, between 35 and 40 mol %, between 40 and 50 mol %, including any range between.

In some embodiments, a molar concentration of the sterol within the nanoparticle is between 20 and 60 mol %, between 20 and 30 mol %, between 20 and 50 mol %, between 30 and 60 mol %, between 20 and 30 mol %, between 30 and 50 mol %, between 50 and 60 mol %, including any range between.

In some embodiments, a weight ratio between the sterol and the phospholipid within the shell is between 1:1 and 1:10, between 1:1 and 1:2, between 1:2 and 1:3, between 1:3 and 1:4, between 1:4 and 1:5, between 1:5 and 1:7, between 1:7 and 1:10, including any range between.

In some embodiments, a molar ratio between the sterol and the phospholipid within the shell is between 1:1 and 1:10, between 1:1 and 1:2, between 1:2 and 1:3, between 1:3 and 1:4, between 1:4 and 1:5, between 1:5 and 1:7, between 1:7 and 1:10, including any range between.

In some embodiments, the shell comprises a first modified lipid and an additional lipid, wherein the first modified lipid is covalently bound to a targeting moiety. In some embodiments, the first modified lipid is covalently bound to a targeting moiety via a spacer, wherein the spacer is as described herein above. In some embodiments, the first modified lipid comprises a lipid-bound to a spacer. In some embodiments, the spacer is a polymer (e.g., a biocompatible polymer such as PEG), wherein the polymer is as described herein. In some embodiments, the first modified lipid comprises a lipid-bound to a polymer, wherein the polymer is further bound to the targeting moiety. In some embodiments, the first modified lipid comprises a lipid-bound to the targeting moiety via a polymer. In some embodiments, the polymer is PEG or a PEG derivative.

In some embodiments, the targeting moiety is as described herein above (e.g., comprising a small molecule or a protein having a binding affinity to a CNS receptor). In some embodiments, the first modified lipid is or comprises one or more conjugates of the invention.

In some embodiments, the additional lipid comprises a chemically modified lipid (e.g., a chemically modified phospholipid). In some embodiments, the additional lipid comprises a lipid covalently bound to a polymer, wherein the polymer is as described herein. In some embodiments, the polymer is alkylated PEG, such as methoxy polyethylene glycol (mPEG) or a derivative thereof.

In some embodiments, the additional lipid comprises a PEG-ylated lipid. In some embodiments, the PEG-ylated lipid comprises a polyethyleneglycol (PEG) moiety covalently bound to the lipid molecule and/or to a derivative thereof. In some embodiments, the PEG-ylated lipid comprises a PEG-modified lipid. In some embodiments, the terms “PEG-ylated lipid” and “PEG-modified lipid” are used herein interchangeably.

In some embodiments, the PEG-ylated lipid comprises PEG-moiety covalently bound to the polar group of the lipid. In some embodiments, the polar group of the lipid (e.g., PE) comprises an amine. In some embodiments, the additional lipid comprises a PEG-ylated PE. In some embodiments, the PEG-moiety is covalently bound to the amine group via one or more linkers, such as the C1-C10alkyl linker or any other linker or functional group capable of covalently binding the PEG moiety to the lipid (e.g., PE). In some embodiments, the PEG-moiety is covalently bound to the amine group (e.g., ethanolamine group of PE) via an amide bond. In some embodiments, the PEG-moiety comprises PEG and/or a derivative thereof. In some embodiments, the PEG derivative comprises a PEG-modified with an alkyl (e.g., C1-C10 alkyl, for example, methyl) or an alkyl derivative at the terminal end of the PEG chain. In some embodiments, the PEG-moiety comprises an alkylated PEG (e.g., comprising C1-C10 alkyl modified PEG). In some embodiments, the additional lipid comprises a lipid (e.g., PE) covalently bound to m-PEG.

In some embodiments, the additional lipid and the first modified lipid are pegylated lipids, wherein the first modified lipid is further bound to the targeting moiety via the PEG chain. In some embodiments, the terms “second modified lipid” and “additional lipid” are used herein interchangeably.

In some embodiments, the PEG-moiety of the first modified lipid is characterized by a molecular weight (MW) ranging between 750Da to about 20,000Da, between 1000 and 10.000 Da, between 1000 and 5000 Da, between 5000 and 10,000 Da, between 1000 and 1500 Da, between 1500 and 2000 Da, between 2000 and 2500 Da, between 2500 and 3000 Da, between 3000 and 5000 Da, including any range between.

In some embodiments, the PEG-moiety of the first modified lipid is characterized by MW of at least 1000 Da, at least 1500 Da, at least 2000 Da, at least 2500 Da, or more, including any range between.

In some embodiments, the PEG-moiety of the additional lipid is characterized by a MW ranging between 300 and 1000 Da, between 300 and 400 Da, between 400 and 500 Da, between 500 and 700 Da, between 700 and 1000 Da, between 500 and 900 Da, between 700 and 900 Da, between 300 and 800 Da, between 300 and 900 Da, between 900 and 950 Da, between 950 and 990 Da, between 300 and 990 Da, including any range between.

In some embodiments, the PEG-moiety of the additional lipid is characterized by a MW of less than 1000, less than 990, less than 950, less than 900, or less than 800, including any range between.

In some embodiments, the MW of the PEG-moiety of the first modified lipid is greater than the MW of the PEG-moiety of the additional lipid. In some embodiments, a MW ratio between the PEG-moiety of the first modified lipid and the PEG-moiety of the additional lipid is between 3:1 and 1.5:1, between 3:1 and 2.5:1, between 2.5:1 and 2:1, between 2:1 and 1.5:1, including any range between.

The targeting moiety needs to be maintained at a predetermined distance from the lipid layer in order to allow binding between the targeting moiety and the CNS receptor within the brain, so as to induce internalization of the nanoparticle into the brain via the BBB. Moreover, it is postulated, that upon crossing the BBB, the nanoparticle will internalize into a nerve cell of interest within the brain (e.g., a neuron or a glia cell). In some embodiments, the cell is an abnormal cell (e.g., an inflamed cell, a cancerous cell, and/or a cell characterized by an abnormal biological activity). In some embodiments, the cell of interest is located within damaged tissue, wherein a damaged tissue refers to any tissue damage caused by a disease and/or disorder, as described herein.

The PEG-moiety of the additional lipid (functioning inter alia as a spacer) is configured to induce steric hindrance, and as a consequence, it is expected to provide a distance between the neighboring targeting moieties on top of the nanoparticle. It is presumed that PEG-moieties of the additional lipid prevent the targeting moieties from collapsing into the lipid layer, thus forming aggregates. It is postulated that such aggregates would be less effective in terms of receptor binding and, as a consequence, would result in poor BBB penetration of the nanoparticles. The inventors presume that in order to facilitate BBB crossing, the targeting moieties need to be distanced from each other and from the lipid layer and/or from the spacers (e.g., PEG-moieties) of the additional lipids. Accordingly, it is presumed that the MW of the spacer (e.g., PEG) of the first modified lipid has to be greater than the MW of the spacer (e.g., PEG) of the additional lipid. In some embodiments, a MW ratio between the PEG-moiety of the first modified lipid and the PEG-moiety of the additional lipid between 3:1 and 1.5:1 is beneficial for the ability of the nanoparticles of the invention to cross BBB, thereby allowing targeted delivery of the bioactive molecule into the brain of a subject in need thereof.

In some embodiments, the nanoparticles of the invention are for use in a targeted delivery of the active agent to a predetermined site (i.e. the target site) within the body of the subject (e.g. CNS, such as brain). In some embodiments, the predetermined site is a tissue, and/or an organ of interest. In some embodiments, the nanoparticles of the invention are characterized by an enhanced accumulation within the predetermined site, wherein enhanced is by at least 50%, at least 2, 3, 4, 5, 6, 8, 10, 50, 100, 1000 times greater concentration of the nanoparticles at the target site, compared to a control location. In some embodiments, the control location is any tissue which is not the target site, the liver, and/or kidney.

Furthermore, it is presumed that the PEG-moieties contribute to the increased blood circulation time of the nanoparticle of the invention and promote internalization within one or more cells of interest.

In some embodiments, a molar concentration of the first modified lipid or of the additional lipid within the shell is between 0.1 and 5 mol %, between 0.1 and 0.5 mol %, between 0.5 and 5 mol %, between 0.5 and 1 mol %, between 1 and 5 mol %, between 1 and 2 mol %, between 2 and 3 mol %, between 3 and 5 mol %, between 1 and 3 mol %, between 2 and 5 mol %, including any range between.

In some embodiments, a molar concentration of the PEG-ylated lipid within the nanoparticle is between 0.1 and 10 mol %, between 0.1 and 0.5 mol %, between 0.5 and 5 mol %, between 0.5 and 1 mol %, between 1 and 5 mol %, between 1 and 2 mol %, between 2 and 3 mol %, between 3 and 5 mol %, between 1 and 3 mol %, between 2 and 5 mol %, between 0.5 and 10 mol %, between 0.1 and 10 mol %, between 0.1 and 0.5 mol %, between 0.5 and 1 mol %, between 1 and 5 mol %, between 5 and 10 mol %, between 5 and 7 mol %, between 7 and 10 mol %, including any range between.

In some embodiments, a molar concentration of the first modified lipid within the shell is at most 5%, at most 3%, at most 4%, at most 2.5%, including any range between.

In some embodiments, a molar concentration of the first modified lipid and/or of the additional lipid within the shell is at most 5%, at most 3%, at most 4%, at most 2.5%, including any range between.

In some embodiments, the molar concentration is calculated based on the total lipid content of the nanoparticle (e.g., of the shell), wherein the lipid content refers to the total amount of the phospholipid, the first modified lipid, the additional lipid, and sterol within the nanoparticle of the invention, as described herein.

In some embodiments, a molar concentration of the conjugate of the invention within the nanoparticle is between 0.1 and 10 mol %, between 0.1 and 0.5 mol %, between 0.5 and 5 mol %, between 0.5 and 1 mol %, between 1 and 5 mol %, between 1 and 2 mol %, between 2 and 3 mol %, between 3 and 5 mol %, between 1 and 3 mol %, between 2 and 5 mol %, between 0.5 and 10 mol %, between 0.1 and 10 mol %, between 0.1 and 0.5 mol %, between 0.5 and 1 mol %, between 1 and 5 mol %, between 5 and 10 mol %, between 5 and 7 mol %, between 7 and 10 mol %, including any range between.

In some embodiments, a molar concentration of the conjugate of the invention within the nanoparticle is between10 and 80 mol %, between 10 and 20 mol %, between 20 and 60 mol %, between 10 and 60 mol %, between 20 and 40 mol %, between 40 and 60 mol %, between 60 and 80 mol %, between 0.1 and 80%, between 0.5 and 80%, between 1 and 80%, between 1 and 50%, including any range between.

In some embodiments, a weight ratio between the first modified lipid and the additional lipid within the shell between 2:1 and 1:2, between 3:1 and 2:1, between 2:1 and 1:1, between 1:1 and 1:2, between 1:2 and 1:3, between 1:1.5 and 1.5:1, including any range between.

In some embodiments, a molar ratio between the first modified lipid and the additional lipid within the shell between 2:1 and 1:2, between 3:1 and 2:1, between 2:1 and 1:1, between 1:1 and 1:2, between 1:2 and 1:3, between 1:1.5 and 1.5:1, including any range between.

In some embodiments, a molar ratio between the first modified lipid and the phospholipid within the shell is between 1:200 and 1:8, between 1:200 and 1:8, between 1:100 and 1:8, between 1:100 and 1:50, between 1:50 and 1:20, between 1:20 and 1:10, between 1:10 and 1:8, including any range between.

In some embodiments, the nanoparticles of the invention encapsulate an effective amount (e.g., therapeutically effective amount) of the biologically active agent. In some embodiments, the nanoparticles of the invention are characterized by loading of the biologically active agent (also referred to herein as the drug loading) sufficient for utilizing thereof in the treatment or prevention of disease. In some embodiments, the biologically active agent is or comprises an antibody, and the loading of the antibody within the nanoparticle of the invention is at least 5, at least 10, at least 20, at least 30, at least 40, at least 50, at least 100, at least 200 antibody units per a single nanoparticle (e.g., liposome), including any range between.

In some embodiments, the biologically active agent comprises a pharmaceutically active agent (e.g., a drug) and/or a diagnostic agent (e.g., a labeling agent). In some embodiments, the composition of the invention comprises an effective amount (e.g., therapeutically effective amount) of the biologically active agent. In some embodiments, the composition of the invention is a pharmaceutical composition comprising a therapeutically effective amount of biologically active agent, as described herein.

As used herein, the term “a therapeutically active agent” describes a chemical substance, which exhibit a therapeutic activity when administered to a subject. As used herein, the term “biologically active agent”, or “bioactive agent”, describes a chemical or a biological substance, which exhibits a biological or physiological activity in an organism.

As used herein, a “therapeutically effective amount” or “an amount effective” refers to an amount effective, at dosages and for periods of time necessary, to achieve the desired therapeutic result. The therapeutically effective amount of the therapeutic agent will depend on the nature of the disorder or condition and on the particular agent and can be determined by standard clinical techniques known to a person skilled in the art.

As used herein, the term “labeling agent” refers to a detectable moiety or a probe and includes, for example, chromophores, fluorescent compounds, phosphorescent compounds, heavy metal clusters, and radioactive labeling compounds, as well as any other known detectable moieties. Also included are contrast agents, e.g., a magnetic resonance imaging (MRI) contrast agent, a computed tomography (CT) contrast agent, a single photon emission computed tomography (SPECT) contrast agent, a positron emission tomography (PET) contrast agent, a bioluminescence (BL) contrast agent, an optical contrast agent, an X-ray contrast agent, and an ultrasonic contrast agent.

The term “radioactive agent” describes a substance (i.e., radionuclide or radioisotope) which loses energy (decays) by emitting ionizing particles and radiation. When the substance decays, its presence can be determined by detecting the radiation emitted by it. For these purposes, a particularly useful type of radioactive decay is positron emission. Exemplary radioactive agents include 99mTc, 18F, 67Ga, 131I and 125I.

In some embodiments, the biologically active agent is a hydrophobic and/or a hydrophilic agent. In some embodiments, the biologically active agent comprises a medicament suitable for treating a disease.

Non-limiting examples of therapeutically active agents that can be beneficially used in embodiments of the present invention include, without limitation, one or more of an anti-inflammatory drug, an anti-proliferative drug, polynucleotide, an antisense oligonucleotide, RNA (e.g. oligo RNA, siRNA, micro-RNA, mRNA and modified RNA), DNA, a chemotherapeutic drug, a terpene, a cannabinoid, an agonist agent, an amino acid agent, an analgesic agent, an antagonist agent, an antibiotic agent, an antibody agent, an antidepressant agent, an antigen agent, an antihistamine agent, an anti-hypertensive agent, an anti-metabolic agent, an antimicrobial agent, an antioxidant agent, a radical (or ROS) scavenging agent, a co-factor, a cytokine, a drug, an enzyme, a growth factor, a heparin, a hormone, an immunoglobulin, an inhibitor, a ligand, a nucleic acid, an oligonucleotide, a peptide, a phospholipid, a prostaglandin, a protein, a toxin, a vitamin and any combination thereof. In some embodiments, the biologically active agent comprises a radical (or ROS) scavenging agent, specifically one or more ionizing radiation protecting agents (e.g., ascorbic acid, cinnamic acid, polyphenols, polyunsaturated compounds, carotenoids, etc.).

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, the term “polynucleic acid” and the term “polynucleotide” are used herein interchangeably. In some embodiments, the polynucleotide comprises 60 to 15000 nucleobases, 15000 to 10000, 10000 to 4700, 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 20 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, a specific polynucleotide comprises a plurality of polynucleotide molecules harboring the same or an identical nucleic acid sequence. In some embodiments, a specific polynucleotide comprises a plurality of polynucleotide molecules harboring essentially the same nucleic acid sequence.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, at least 3 nucleobase, at least 5 nucleobase, at least 7 nucleobase, or at least 10 nucleobases, or any value and range therebetween. Each possibility represents a separate embodiment of the invention.

In some embodiments, a polynucleotide comprises RNA, DNA, a synthetic analog of RNA, a synthetic analog of DNA, DNA/RNA hybrid, or any combination thereof. In some embodiments, a nanoparticle of the invention comprises a polynucleotide selected from: RNA, DNA, a synthetic analog of RNA, a synthetic analog of DNA, DNA/RNA hybrid, or any combination thereof.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest. In some embodiments, a RNA polynucleotide of an mRNA encodes 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5- 6, 6-10, 6-9, 6-8, 6-7, 7-10, 7-9, 7-8, 8-10, 8-9 or 9-10 polypeptides. In some embodiments, a RNA polynucleotide of an mRNA encodes at least 10, 20, 30, 40, 50, 60, 70, 80, 90 or 100 polypeptides. In some embodiments, a RNA polynucleotide of an mRNA encodes at least 100 or at least 200 polypeptides.

In some embodiments, the nucleic acids are therapeutic mRNAs. As used herein, the term “therapeutic mRNA” refers to an mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

Thus, the carrier of the invention can be used as therapeutic or prophylactic agent. They are provided for use in medicine. For example, the polynucleotide encapsulated within the carrier described herein (e.g. LNP) can be administered to a subject, wherein the polynucleotide is translated in vivo to produce a therapeutic peptide.

In some embodiments, the polynucleotide comprises an inhibitory nucleic acid.

In some embodiments, the polynucleotide comprises an antisense oligonucleotide.

As used herein, an “antisense oligonucleotide” refers to a nucleic acid sequence that is reversed and complementary to a DNA or RNA sequence. It is assumed that, antisense oligonucleotides sterically block a specific DNA or RNA sequence, thereby prevent or at least partially inhibit transcription and/or translation of the specific DNA or RNA sequence, respectively. Exemplary antisense oligonucleotides include a DNA and/or RNA sequence, or comprises a chemically modified backbone/and or base modification within the sequence. Exemplary chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid (PNA) backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone, tricyclo-DNA (tcDNA) nucleic acid backbone, ligand-conjugated antisense, and a combination thereof.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory nucleic acid has one or more chemical modifications to the backbone or side chains. In some embodiments, the inhibitory nucleic acid has at least one chemically modified nucleotide (e.g. LNA, and/or a phosphorothioate).

Non-limiting examples of inhibitory nucleic acids useful according to the herein disclosed invention include, but are not limited to: ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)-of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein.

In some embodiments, the biologically active agent comprises a therapeutic agent for the treatment of one or more brain disease(s). In some embodiments, the brain disease is as described herein.

The term “cancer”, as used herein, refers to a disease or disorder resulting from the proliferation of ontogenically transformed cells. Examples of particular cancers that may be treated according to the method of the present invention include oral cancer, such as oral squamous cell carcinoma, and oral pharyngeal cancer.

The phrase “anticancer agent” or “anticancer drug”, as used herein, describes a therapeutically active agent that directly or indirectly kills cancer cells or directly or indirectly inhibits, stops, or reduces the proliferation of cancer cells. Anti-cancer agents include those that result in cell death and those that inhibit cell growth, proliferation and/or differentiation. In some embodiments, the anti-cancer agent is selectively toxic against certain types of cancer cells but does not affect or is less effective against normal cells. In some embodiments, the anti-cancer agent is a cytotoxic agent.

Examples of cancer therapeutic agents include, e.g., but are not limited to Abiraterone, Acitretin, Aldesleukin, Alemtuzumab, Amifostine, Amsacrine, Anagrelide, Anastrozole, Arsenic, Asparaginase, Asparaginase Erwinia, Axitinib, azaCITItidine, BCG, Bendamustine, Bevacizumab, Bexarotene, Bicalutamide, Bleomycin, Bortezomib, Brentuximab, Bromocriptine, Buserelin, Busulfan, Cabazitaxel, Cabergoline, Capecitabine, CARBOplatin, Carmustine, Cetuximab, Chlorambucil, CISplatin, Cladribine, Clodronate, Crizotinib, Cyclophosphamide, CycloSPORINE, Cytarabine, Dacarbazine, Dactinomycin, Dasatinib, DAUNOrubicin, Degarelix, Denosumab, Dexamethasone, Dexrazoxane, DOCEtaxel, DOXOrubicin, DOXOrubicin pegylated liposomal, Enzalutamide, Epirubicin, Eribulin, Erlotinib, Estramustine, Etoposide, Everolimus, Exemestane, Filgrastim, Fludarabine, Fluorouracil, Flutamide, Fulvestrant, Gefitinib, Gemcitabine, Goserelin, Hydroxyurea, IDArubicin, Ifosfamide, Imatinib, Iniparib, Interferon alfa-2b, Ipilimumab, Irinotecan, Ixabepilone, Lambrolizumab, Lanreotide, Lapatinib, Lenalidomide, Letrozole, Leucovorin, Leuprolide, Lomustine, Mechlorethamine, medroxyPROGESTERone, Megestrol, Melphalan, Mercaptopurine, Mesna, Methotrexate, mitoMYCIN, Mitotane, mitoXANTRONE, Nilotinib, Nilutamide, Octreotide, Ofatumumab, Oxaliplatin, PACLitaxel, ACLitaxel nanoparticle, albumin-bound (nab), Pamidronate, Panitumumab, Pazopanib Pemetrexed, Pertuzumab, Porfimer, Procarbazine, Quinagolide, Raltitrexed, Reovirus Serotype 3—Dearing Strain, riTUXimab, Romidepsin, Ruxolitinib, SORAfenib, Streptozocin, SUNItinib, Tamoxifen, Temozolomide, Temsirolimus, Teniposide, Testosterone, Thalidomide, Thioguanine, Thiotepa, Thyrotropin alfa, Tocilizumab, Topotecan, Trastuzumab (HERCEPTIN®), Trastuzumab, Emtansine (KADCYLA®), Treosulfan, Tretinoin, Vemurafenib, vinBLAstine, vinCRIstine and Vinorelbine.

Examples of chemotherapeutic agents used as a therapeutic agent include, e.g., but are not limited to, e.g., alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolites (e.g., methotrexate, 5-fluorouracil, capecitabine, cytarabine, gemcitabine, fludarabine), topoisomerase interactive agents (e.g., camptothecin, irinotecan, topotecan, ctoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, Casodex), kinase and tyrosine inhibitors (e.g., imatinib (STI571 or Gleevac); gefitinib (Iressa); and erlotinib (Tarceva), etc. See, e.g., Cancer: Principles and Practice of Oncology, 7th Edition, Devita, et al., Lippincott Williams & Wilkins, 2005, Chapters 15, 16, 17, and 63).

Examples of therapeutic antibodies that are FDA approved or in review in the EU or US included, e.g., but are not limited to Lecanemab (target—amyloid beta, indication—Alzheimer's disease, review), Omburtamab (target—B7-H3, indication—CNS/leptomeningeal metastasis from neuroblastoma, review), Aducanumab (target—amyloid beta, indication—Alzheimer's disease, review), Naxitamab (target—GD2, indication—neuroblastoma), Satralizumab (target—IL-6R, indication—neuromyelitis optica and neuromyelitis Optica spectrum disorders), Inebilizumab (target—CD19, indication—neuromyelitis Optica and neuromyelitis Optica spectrum disorders, review), Ocrelizumab (target—CD20, indication—Multiple sclerosis), Dinutuximab (target—GD2, indication—neuroblastoma), and Natalizumab (target—a4 integrin, indication—Multiple sclerosis), etc.

In some embodiments, the liposomes of the invention can be manufactured according to any of the methods known in the art. In some embodiments, a method of manufacturing the liposomes of the invention comprises (a) mixing the components of the lipid layer in an organic solvent such as chloroform, thereby obtaining an organic phase. In some embodiments, the molar ratios between the components of the lipid layer are as described herein above. In some embodiments, a total concertation of the components of the lipid layer within the organic phase is between 50 and 150 mM, between 70 and 150 mM, between 70 and 100 mM, between 90 and 150 mM, between 100 and 150 mM, including any range between. In some embodiments, a concertation of the phospholipid within the organic phase is at most 150 mM, at most 100 mM, at most 140 mM, at most 130 mM, at most 120 mM, including any range between. In some embodiments, the mixing is performed at a temperature above the Tm of the phospholipid.

In some embodiments, the method further comprises (b) removing the organic solvent (e.g., by evaporation), thereby obtaining a solid (e.g., in the form of a thin lipid film). In some embodiments, the method further comprises (c) combining the solid with an aqueous solution comprising the bioactive compound (e.g., an antibody), thereby obtaining an aqueous dispersion. In some embodiments, the method further comprises (d) extruding the aqueous dispersion to obtain a liquid composition comprising a plurality of nanoparticles (e.g., liposomes) of the invention. In some embodiments, the nanoparticles have an average particle size and/or PDI as described hereinabove (e.g., between 80 and 150 nm). In some embodiments, the step d is performed at a temperature below 50° C., below 47° C., below 45° C., below 46° C., including any range between.

In some embodiments, the liposomes of the invention are extruded liposomes. In some embodiments, the liposomes of the invention are extrudable at a temperature below 50° C., below 47° C., below 45° C., below 46° C., including any range between.

### Pharmaceutical Compositions

In another aspect, there is provided a liquid composition comprising a plurality of nanoparticles (e.g., liposomes) of the invention. In some embodiments, the liquid composition is an aqueous solution comprising a plurality of nanoparticles dispersed therewithin. In some embodiments, the nanoparticles are stably dispersed within the liquid composition (e.g., being substantially devoid of: aggregates and/or disintegration of the nanoparticles). In some embodiments, the nanoparticles within the liquid composition are stably encapsulating the bioactive compound (e.g., being substantially devoid of disintegration of the nanoparticles and leakage of the bioactive compound, and/or the bioactive compound substantially maintains its biological activity, as compared to a non-encapsulated bioactive compound). In some embodiments, the liquid composition is stable for at least 24 h, at least 48 h, at least 3 days (d), at least 7 d, at least 30 d, at least 60 d, at least 150 d, at least 1 year (y) including any range between, when stored under normal (or appropriate) storage conditions. In some embodiments, the normal (or appropriate) storage conditions comprise an ambient atmosphere and a temperature between 5 and 45°, between 5 and 15°, between 15 and 45°, between 20 and 45°, including any range between.

In some embodiments, a concertation of the nanoparticles of the invention within the liquid composition (e.g. effective amount) is between 50 and 150 mM, between 70 and 150 mM, between 70 and 100 mM, between 90 and 150 mM, between 100 and 150 mM, including any range between.

In some embodiments, a therapeutically effective amount of the nanoparticles of the invention within the liquid composition is between 1 and 500 nM, between 1 and 10 nM, between 10 and 100 nM, between 100 and 300 nM, between 300 and 500 nM, including any range between.

In some embodiments, the liquid composition is a pharmaceutical composition, comprising a pharmaceutically effective amount of the nanoparticles of the invention and/or a pharmaceutically effective amount of the bioactive compound. In some embodiments, the pharmaceutical composition comprises a plurality of the nanoparticles of the invention and a pharmaceutically acceptable carrier.

As used herein, a “pharmaceutically acceptable formulation,” “pharmaceutical composition,” or “pharmaceutically acceptable composition” may include any of a number of carriers such as solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Pharmaceutical compositions containing the presently described nanoparticles as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

A composition may comprise different types of carriers depending on whether it is to be administered in solid, liquid, or aerosol form and whether it needs to be sterile for such routes of administration as injection. A person of ordinary skill in the art would be familiar with techniques for generating sterile solutions for injection or application by any other route. Sterile injectable solutions are prepared by incorporating the active compounds in the required amount in an appropriate solvent with various other ingredients familiar to a person of skill in the art.

The carrier may comprise, in total, from about 0.1% to about 99.99999% by weight of the pharmaceutical compositions presented herein.

According to some embodiments, the pharmaceutical composition is formulated for systemic administration. According to some embodiments, the pharmaceutical composition is formulated for topical administration.

The compositions contemplated herein may take the form of solutions, suspensions, emulsions, combinations thereof, or any other pharmaceutical acceptable composition as would commonly be known in the art.

In some embodiments, the carrier is a solvent. For a non-limiting example, the composition may be disposed of in the solvent. Such a solvent includes any suitable solvent known in the art, such as water, saline, or phosphate-buffered saline.

The formulation of the composition may vary depending upon the route of administration. For parenteral administration in an aqueous solution, for example, the solution should be suitably buffered if necessary, and the liquid diluent first rendered isotonic with sufficient saline or glucose. Sterile aqueous media which can be employed will be known to those of skill in the art in light of the present disclosure.

Supplementary active ingredients can also be incorporated into the compositions. For human administration, preparations should meet sterility and general safety and purity standards as required by FDA Office of Biologics standards. Administration may be by any known route.

In certain embodiments, a pharmaceutical composition includes at least about 0.01 g to about 5 g of the particle disclosed herein per kilogram of a subject.

The pharmaceutical composition may comprise various antioxidants to retard the oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition must be stable under the conditions of manufacture and storage and preserved against the contaminating action of microorganisms, such as bacteria and fungi.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride, or combinations thereof.

In other embodiments, nasal solutions or sprays, aerosols, or inhalants may be used. Nasal solutions are usually aqueous solutions designed to be administered to the nasal passages in drops or sprays.

Solid compositions for oral administration are also contemplated. In these embodiments, the solid composition may comprise, for example, solutions, suspensions, emulsions, tablets, pills, capsules, sustained release formulations, buccal compositions, troches, elixirs, suspensions, syrups, or combinations thereof.

Sterile injectable solutions are prepared by incorporating the active compounds (e.g., nanoparticles) in the required amount in the appropriate solvent with various other ingredients enumerated above. The liquid medium should be suitably buffered if necessary, and the liquid diluent first rendered isotonic prior to injection with sufficient saline or glucose.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art.

### Therapeutic and Diagnostic Use of the Composition

According to some embodiments, the pharmaceutical composition is for use in the prevention of a disease in a subject in need thereof. According to some embodiments, the pharmaceutical composition is for use in the treatment of a disease in a subject in need thereof. In some embodiments, there is provided a method for treating or reducing at least one symptom associated with the disease in the subject, the method comprising administering a therapeutically effective amount of the pharmaceutical composition of the invention to the subject. In some embodiments, the method is for a targeted delivery of the active agent to the target site within the body of the subject, as disclosed herein. In some embodiments, the targeted delivery is so as to induce an enhanced accumulation of the active agent within the target site, wherein enhanced is as described herein.

In some embodiments, the therapeutically effective amount (or dose) comprises between 0.1 and 100 mg, between 1 and 100 mg, between 1 and 50 mg per day, by dry weight of the nanoparticles of the invention, including any range between.

In some embodiments, the disease is a central nervous system disease. According to some embodiments, the disorder is a brain disorder. In some embodiments, the disease is a neurodegenerative disease and/or a neuroinflammatory disorder. In some embodiments, the disease is Alzheimer's disease. In some embodiments, the disease is selected from Alzheimer's Disease, multiple sclerosis, dementia, Parkinson's disease (PD), Huntington's disease, Down syndrome, Amyotrophic lateral sclerosis (ALS), and prion disease or any combination thereof. According to some embodiments, the disease is Huntington's disease, spinocerebellar ataxia, amyotrophic lateral sclerosis, Friedreich's ataxia, and motor neuron disease (Lou Gehrig's disease) or spinal muscular atrophy. According to some embodiments, the disease is a prion disease. In some embodiments, the disease is a proliferative disease. In some embodiments, the disease is cancer.

In some embodiments, the pharmaceutical composition is for use in the prevention or treatment of a cognitive disorder. In some embodiments, the pharmaceutical composition is for use in the amelioration of a condition associated with a cognitive disorder. In some embodiments, the pharmaceutical composition is for use in improving cognitive function or inhibiting cognitive disfunction of the subject.

As used herein, the term “cognitive function” is well-known in art and refers to multiple mental abilities, including learning, thinking, reasoning, remembering, problem-solving, decision making, and attention.

In some embodiments, the disease is selected from brain cancer, epilepsy, and other seizure disorders, mental disorders, stroke and Transient Ischemic Attack (TIA) and central nervous system (CNS) diseases, and a neurodegenerative disease

As used herein, the term “subject” refers to any animal (e.g., a mammal), including, but not limited to, humans, non-human primates, rodents, and the like (e.g., which is to be the recipient of a particular treatment). Typically, the terms “subject” and “patient” are used interchangeably unless indicated otherwise herein.

In some embodiments, the subject is a human subject. In some embodiments, the subject is at risk of being afflicted with a disease, a disorder, or a medical condition. In some embodiments, the subject is diagnosed with a disease, a disorder, or a medical condition. In some embodiments, the subject is diagnosed with a genetic disorder. In some embodiments, the subject is at risk of being afflicted with a neurodegenerative disease. In some embodiments, the subject is diagnosed with a neurodegenerative disease. In some embodiments, the subject is diagnosed with Alzheimer's disease. In some embodiments, the subject is diagnosed with Parkinson's disease.

As used herein, a subject at risk of being afflicted with a disease, a disorder, or a medical condition, is a subject that presents one or more signs or symptoms indicative of a disease, a disorder, or a medical condition or is being screened for a disease, a disorder, or a medical condition (e.g., during a routine physical). A subject at risk of being afflicted with a disease, a disorder, or a medical condition, may also have one or more risk factors. A subject at risk of being afflicted with a disease, a disorder, or a medical condition encompasses an individual that has not been previously tested for the disease, disorder, or medical condition. However, a subject at risk of being afflicted with a disease, a disorder, or a medical condition, also encompasses an individual who has received a preliminary diagnosis but for whom a confirmatory test (e.g., biopsy and/or histology) has not been done or for whom the stage of the disease, disorder, or medical condition is not known. The term further includes people who once had the disease, disorder, or medical condition (e.g., an individual in remission).

A subject at risk of being afflicted with a disease, disorder, or medical condition may be diagnosed as having or alternatively found not to have the disease, disorder, or medical condition.

As used herein, a subject diagnosed with a disease, disorder, or medical condition, may be diagnosed using any suitable method, including but not limited to biopsy, x-ray, blood test, and the diagnostic methods of the present invention. A “preliminary diagnosis” is one based only on visual (e.g., CT scan or the presence of a lump) and antigen tests.

In some embodiments, the subject is afflicted with a disease, disorder, or medical condition, and the imaging method is used for determining the stage of the disease, disorder, or medical condition. In some embodiments, the subject afflicted with a disease, disorder, or medical condition, was treated with a drug, and the imaging method is used for follow-up of the treatment.

As used herein, the terms “treatment”, “treating”, or “ameliorating” of a disease, disorder, or condition, refer to the alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the present invention provides for a method of administering a biologically active agent for the prevention or treatment of a disease in a subject in need thereof, the method comprising administering to the subject the pharmaceutical composition described herein.

In some embodiments, the present invention provides a theranostic method. The method comprises the steps of administering to a subject in need thereof the pharmaceutical composition of the invention and imaging a target site of the subject to determine whether the nanoparticles accumulated in the target site of the subject. In some embodiments, the target site is a site in the brain of the subject.

In some embodiments, administering the pharmaceutical composition to the subject can be done by using any method known to those of ordinary skill in the art. The mode of administering may vary based on the application. For example, the mode of administration may vary depending on the particular cell, tissue, organ, portion of the body, or subject to be imaged. For example, administering the composition may be done intravenously, intracerebrally, intracranially, intrathecally, intracerebroventricular, into the substantia nigra or the region of the substantia nigra, intradermally, intraarterially, intraperitoneally, intralesionally, intratracheally, intranasally, intramuscularly, intraperitoneally, subcutaneously, orally, topically, locally, by inhalation (e.g., aerosol inhalation), by injection, by infusion, by continuous infusion, by localized perfusion bathing target cells directly, via a catheter, via a lavage, or by other method or any combination of the forgoing as would be known to one of ordinary skill in the art.

In some embodiments, the pharmaceutical composition is administered intravenously.

Upon formulation, compositions will be administered in a manner compatible with the dosage formulation and in such amount as is effective. For example, the nanoparticles may be administered in such an amount that is effective for a particular imaging application desired.

An effective amount of the pharmaceutical composition is determined based on the intended goal, for example, based on the imaging method and the subject or portion of a subject to be imaged. The quantity to be administered may also vary based on the particular route of administration to be used. The composition is preferably administered in a “safe and effective amount.” As used herein, the term “safe and effective amount” refers to the quantity of a composition which is sufficient for the intended goal (e.g., imaging) without undue adverse side effects (such as toxicity, irritation, or allergic response).

In some embodiments, imaging of the target site is performed by an imaging technique that utilizes penetrating radiation. According to some embodiments, the imaging technique is selected from the group comprising of magnetic resonance imaging (MRI), computed tomography imaging (CT), X-ray imaging, positron emission tomography (PET), single-photon emission computed tomography (SPECT), and ultrasound (US).

In some embodiments, the imaging step is performed 0.5 to 96 hours post the administering step.

In some embodiments, the method comprises the step of determining whether the nanoparticles accumulated in the target site of the subject. In some embodiments, treatment decision may be not to administer a therapy. In some embodiments, the analysis of the imaging data is used for deciding on a route of treatment adequate to the patient. In some embodiments, deciding on a route of treatment adequate to the patient depends, for example, on the stage of the disease, disorder, or medical condition, as well as on the health state of the patient. In some embodiments, the route of treatment includes one or more protocols of treatment selected from the group comprising of: intravenous, intranasal, intraperitoneal, intramuscular and subcutaneous, and any other biological or inorganic product intended for treatment. In some embodiments, a treatment is administered subsequent to the imaging. In some embodiments, a treatment is administered to the subject in real-time while imaging the subject.

In some embodiments, imaging and treating the subject are performed simultaneously. In some embodiments, the biologically active molecule may be activated in the subject target site subsequent to imaging.

### Kits

In some embodiments, the invention provides kits comprising one or more compositions disclosed herein. In some embodiments, the invention provides kits useful for methods disclosed herein. For example, a kit may include a container having a sterile reservoir that houses any composition disclosed herein. In some embodiments, the kit further includes instructions. For example, a kit may include the instructions for administering the composition to a subject (e.g., indication, dosage, methods, etc.). In yet another example the kit may include instructions of to apply the compositions and methods of the invention to imaging systems e.g., computed tomography (CT), ultrasound (US), magnetic resonance imaging (MRI).

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Any concentration ranges, percentage range, or ratio range recited herein are to be understood to include concentrations, percentages or ratios of any integer within that range and fractions thereof, such as one-tenth and one-hundredth of an integer, unless otherwise indicated.

Any number range recited herein relating to any physical feature, such as polymer subunits, size, or thickness, are to be understood to include any integer within the recited range unless otherwise indicated.

As used herein, the term “about,” when combined with a value, refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, a reference to “a polynucleotide” includes a plurality of such polynucleotides, and a reference to “the polypeptide” includes a reference to one or more polypeptides and equivalents thereof known to those skilled in the art, and so forth. It is further noted that the claims may be drafted to exclude any optional element. As such, this statement is intended to serve as an antecedent basis for the use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### General

In some embodiments, the term “substituted” comprises more or more (e.g. 2, 3, 4, 5, 6, or more) substituents, wherein the substituent(s) is as described herein. The term “substituent”, as used herein comprises one or more substituents (e.g. 1, 2, 3, 4, 5, or 6), each independently selected from the group consisting of: C₁-C₆ alkyl, halo, —NO₂, —CN, —OH, —NH₂, carbonyl, —CONH₂, —CONR′₂, —CNNR₂, —CSNR₂, —CONH—OH, —CONH—NH₂, —NHCOR′, —NHCSR′, —NHCNR′, —NC(—O)OR′, —NC(—O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R′, —OCOR′, —OCOR′, —OC(═O)OR′, —OC(═O)NR′, —OC(═S)OR′, —OC(═S)NR′, a heteroatom, an optionally substituted cycloalkyl, an optionally substituted heterocyclyl, or a combination thereof, wherein each R′ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom).

As used herein, the term “alkyl” describes an aliphatic hydrocarbon including straight chain and branched chain groups. In some embodiments, the alkyl group has 1 to 20 carbon atoms, between 1 and 10, between 1 and 5, between 5 and 10, between 10 and 15, between 15 and 20, including any range between.

In some embodiments, the alkyl encompasses a short alkyl and/or a long alkyl. In some embodiments, the alkyl has from 21 to 100 carbon atoms, or more. In the context of the present invention, a “long alkyl” is an alkyl having at least 20 carbon atoms in its main chain (the longest path of continuous covalently attached atoms). A short alkyl therefore has 20 or less (e.g. 2, 3, 4, 5, 6, 8, 10, 15, or 20) main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

In some embodiments, R′ and R″ are the same or different, wherein each of R′ and R″ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N)NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O-C1-6-alkyl, C1-6-alkyl, hydroxy-C1-6-alkyl and amino-C1-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun, but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion, unless otherwise stated, adjectives such as “substantially” and “about” modifying a condition or relationship characteristic of a feature or features of an embodiment of the invention are understood to mean that the condition or characteristic is defined within tolerances that are acceptable for the operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Various embodiments and aspects of the present invention, as delineated herein above and as claimed in the claims section below, find experimental support in the following examples.

### EXAMPLES

Generally, the nomenclature used herein and the laboratory procedures utilized in the present invention include molecular, biochemical, microbiological, and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Synthetic Procedures of the Exemplary Conjugates of the Invention

### Synthesis of DSPE-PEG1000-Caffeine:

### Synthesis of Theophylline-7-Acetic Acid (Derivate of Caffeine)-NHS:

To 32 mg of trans-4-cotininecarboxylic acid (0.145 mmol) 7 ml of dimethylformamide (DMF) (6 ml) and chloroform (1 ml) were added followed by addition of 63.4 mg N-Hydroxysuccinimide (NHS) (0.551 mmol). To the reaction solution, 105.5 mg of 1-Ethyl-3-(3-dimethylaminopropyl)carbodiimide (EDC) (0.550 mmol) was added, and the reaction was stirred for 4 hours at ambient temperature before completion. The reaction was tested by TLC (TLC mobile phase: 75% chloroform: 25% methanol; exposure by UV light). In order to clean the product from the other reagents, the reaction solution was purified by a flash chromatography system. The product was eluted with 21% methanol: 79% chloroform. The pure product fractions were combined and evaporated to dryness. 23 mg pure product was obtained and confirmed by TLC.

### Synthesis of DSPE-PEG1000-Caffeine:

To 23 mg of Theophylline-7-acetic acid (derivate of caffeine)-NHS (0.0686 mmol) 2 ml dichloromethane and 1 ml dimethylformamide were added, followed by the addition of 200 μl triethylamine. To the reaction solution, 60 mg of DSPE-PEG1000 (0.0338 mmol) was added, and the reaction was stirred overnight at ambient temperature till completion, as was observed by TLC (TLC mobile phase: 75% chloroform, 25% methanol; Staining ninhydrin). To the reaction solution, 50 ml of heptane were added (dimethylformamide forms an azeotrope with heptane), and the reaction was evaporated under reduced pressure to dryness by rotovapor followed by purification by a flash chromatography system. The product was eluted with 33% methanol: 67% chloroform. The pure fractions were combined and evaporated to dryness. The obtained product—45.2 mg was identified by H-Nuclear Magnetic Resonance (H-NMR) and TLC. The product was stored in a −80° C. freezer.

### Synthesis of DSPE-PEG1000-GlutarylChloride-Ketamine:

To 1 gr of ketamine hydrochloride (3.65 mmol), 10 ml of water, and 386 mg of anhydrous sodium carbonate (3.65 mmol) were added. To the solution, 200 ml isopropanol was added (water forms an azeotrope with isopropanol), and the solution was evaporated under reduced pressure to dryness by rotovapor. According to TLC, the purity of the ketamine-free base was about 90%. Therefore, the product was used in the next step.

To 50 mg of DSPE-PEG1000 (0.0282 mmol), 50 ml of chloroform was added, followed by the addition of 1 ml triethylamine. The reaction was stirred, and 4.8 mg of glutaryl chloride (0.0283 mmol) was added gradually. The reaction was stirred for 4 hours until completion according to TLC (TLC mobile phase: 20% methanol, 80% chloroform; Staining ninhydrin). Then 32 mg (0.134 mmol) ketamine-free base (see step A) was added to the reaction solution, and all the reagents were stirred overnight at ambient temperature. The reaction solution was tested by Thin-Layer Chromatography—TLC (TLC mobile phase: 20% methanol, 80% chloroform; Staining ninhydrin). The reaction was complete (there was an excess of ketamine-free base and no reactant left after the reaction). The reaction solution was filtered and evaporated to dryness by rotovapor under reduced pressure, followed by purification by a flash chromatography system. The product was eluted with a 12-30% gradient of methanol in chloroform. The pure fractions were combined and evaporated to dryness. The obtained product—59.4 mg was confirmed by H-NMR and TLC. The product was stored in a −80° C. freezer.

### Synthesis of DSPE-PEG1000-Glucose:

Synthesis of D-Glucuronic Acid (Derivate of glucose)-NHS:

To 500 mg of D-Glucuronic acid (2.58 mmol), 5 ml of dimethylformamide was added, followed by the addition of 360 μl triethylamine. To the reaction solution, 661 mg of Di(N-succinimidyl) carbonate (DSC) (2.58 mmol) were added, and the reaction was stirred overnight at 4° C. till completion as was observed by TLC (TLC mobile phase: 50% methanol, 50% chloroform; Staining Phosphomolybdic Acid (PMA)). The reaction solution was diluted with 50 ml of chloroform and filtered. The precipitate was dissolved in 50% methanol and 50% chloroform solution. Then more chloroform solvent was added to get a 10% chloroform, 90% methanol final solution. The new precipitate was filtered, and the solution was evaporated to dryness by rotovapor under reduced pressure. 568 mg of product was obtained, and it contained some traces of regents from the evaporation. Then 5 ml of dimethylformamide were added for dissolving. The product was stored in a −20° C. freezer.

### Synthesis of DSPE-PEG1000-Glucose:

To 100 mg of DSPE-PEG1000 (0.0564 mmol), 7.5 ml of D-Glucuronic acid (derivate of glucose)-NHS (see step A) was added, followed by the addition of 90 μl triethylamine. The reaction was stirred overnight at ambient temperature till completion according to TLC (TLC mobile phase: 20% methanol, 80% chloroform; Staining ninhydrin). 50 ml of heptane were added (dimethylformamide forms an azeotrope with heptane), and the reaction solution was evaporated under reduced pressure to dryness by rotovapor followed by purification by a flash chromatography system. The product was eluted by a 15-50% gradient of methanol in chloroform. The pure fractions were combined and evaporated to dryness. The obtained product—121.6 mg was identified by H-NMR and TLC. The product was stored in a −80° C. freezer.

### Synthesis of DSPE-PEG1000-γ-Aminobutyric Acid (GABA):

Synthesis of N-tert-butoxycarbonyl-4-aminobutyric Acid (BOC-GABA):

To 1 gr of GABA (9.7 mmol), 20 ml of 50% acetone and 50% water solution were added, followed by the addition of 2.8 ml triethylamine. Then 2.4 gr of di-tert-butyl decarbonate (11.0 mmol) in a solution of 5 ml acetone was added gradually to the reaction solution that was stirred. All the reagents in the solution were stirred overnight at ambient temperature till completion according to TLC (TLC mobile phase: 15% methanol, 85% chloroform; Staining bromocresol green). The reaction solution was evaporated by rotovapor under reduced pressure until removing all the organic component solvent. To the aqueous residue, dilute hydrochloric acid (HCl) solution was added until pH was changed to a 4-5 value. Then the solution was extracted with ethel acetate (3 times). All the organic fractions were combined and washed with NaCl solution. The residue was dried over anhydrous magnesium sulfate. The residue was evaporated to dryness by rotovapor under reduced pressure, filtered with acetonitrile, and finally evaporated again to dryness. The obtained solid—1048.4 mg. According to TLC, the purity was about 94%. Therefore, the product was used in the next step. The product was kept at 4° C.

### Synthesis of BOC-GABA-NHS:

To 1 gr of crude BOC-GABA (4.92 mmol) 25 ml of acetonitrile were added, followed by the addition of 0.680 gr NHS (5.90 mmol). To the reaction solution, 1.23 gr of EDC (6.40 mmol) was added, and the reaction was stirred for 2 hours at ambient temperature before completion. The reaction was tested by TLC (TLC mobile phase: 3% methanol, 97% chloroform; Staining PMA and exposure by UV light). The reaction solution was diluted in 130 ml of diethyl ether. Then the solution was extracted with water (2 times—25 ml). The organic phase was dried over anhydrous magnesium sulfate, filtered, and evaporated to dryness by rotovapor under reduced pressure. The obtained solid—1250 mg. According to TLC, the purity was about 95%. Therefore the product was used in the next step. The product was kept at 4° C.

### Synthesis of DSPE-PEG1000-GABA-BOC:

To 100 mg of DSPE-PEG1000 (0.0564 mmol), 3 ml of chloroform were added, followed by the addition of 200 μl triethylamine. To reaction, 101 mg of crude BOC-GABA-NHS (0.335 mmol) were added, and the reaction was stirred overnight at ambient temperature till completion according to TLC (TLC mobile phase: 22% methanol, 78% chloroform; Staining ninhydrin). The reaction solution was purified by a flash chromatography system. The product was eluted with 15% methanol and 85% chloroform. Pure fractions (>80% purity by TLC) were combined and evaporated to dryness to give 160 mg of DSPE-PEG1000-GABA-BOC that was used as is the next step/steps.

### Synthesis of DSPE-PEG1000-GABA:

To 160 mg of crude DSPE-PEG1000-GABA-BOC, 2.5 ml of chloroform were added, followed by the addition of 200 μl Trifluoroacetic acid (TFA. The reaction solution was evaporated under reduced pressure to dryness by rotovapor followed by purification by a flash chromatography system. The product was eluted with 25% methanol: and 75% chloroform. The pure fractions were combined and evaporated to dryness. The obtained product—77.3 mg was identified by H-NMR and TLC. The product was stored in a −80° C. freezer.

### Synthesis of DSPE-PEG1000-Cotinine (Derivate of Nicotine):

Synthesis of trans-4-cotininecarboxylic Acid (Derivate of Cotinine)-NHS:

To 500 mg of trans-4-cotininecarboxylic acid (2.27 mmol) 20 ml of dimethylformamide were added, followed by the addition of 950 μl triethylamine. To the reaction solution, 640 mg of DSC (2.50 mmol) were added, and the reaction was stirred overnight at ambient temperature in an inert environment till completion, as was observed by TLC (TLC mobile phase: 25% methanol, 75% chloroform; Staining ninhydrin). The reaction solution was kept in the freezer and used in the next step.

### Synthesis of DSPE-PEG1000-Cotinine

To 100 mg of DSPE-PEG1000 (0.0564 mmol), 1 ml of chloroform was added, followed by the addition of 3 ml trans-4-cotininecarboxylic acid-NHS solution. The reaction was stirred for 1 hour at ambient temperature till completion, as was observed by TLC (TLC mobile phase: 25% methanol, 75% chloroform; Staining ninhydrin). Then 50 ml of heptane were added (dimethylformamide forms an azcotrope with heptane), and the solution was evaporated under reduced pressure to dryness by rotovapor followed by purification by a flash chromatography system. The product was eluted with 15% methanol: 85% chloroform. The pure fractions were combined and evaporated to dryness. To the obtained solid—150.5 mg 5 ml of diethyl ether were added gradually, and the reaction was stirred. Then the solution was centrifugated, and the liquid phase evaporated to dryness. The obtained product—137.2 mg was tested by TLC and H-NMR. The product was stored in a -80° C. freezer.

### Synthesis of DSPE-PEG1000-D-Aminosuccinic Acid (Aspartic Acid):

Synthesis of N-BOC-D-aspartic acid-1-tert-butyl ester-NHS:

To 300 mg of N-BOC-D-aspartic acid-1-tert-butyl ester (1.04 mmol), 4 ml of dichloromethane were added, followed by the addition of 144 μl triethylamine. To the reaction solution, 266 mg of DSC (1.04 mmol) were added, and the reaction was stirred overnight at ambient temperature in an inert environment. The reaction was tested by TLC (TLC mobile phase: 5% methanol, 95% chloroform; Staining ninhydrin). The reaction solution was evaporated under reduced pressure to dryness by rotovapor followed by purification by a flash chromatography system. The product was eluted by a gradient of 0-2% methanol in chloroform. The best product fractions were combined and evaporated to dryness. The obtained product—547 mg was used as is in the next step.

Synthesis of DSPE-PEG1000-N-BOC-D-aspartic acid-1-tert-butyl ester:

To 100 mg of DSPE-PEG1000 (0.0564 mmol), 3 ml chloroform was added, followed by the addition of 150 mg of crude N-BOC-D-aspartic acid-1-tert-butyl ester-NHS (see step A) and 250 μl of triethylamine. The reaction mixture was stirred overnight at ambient temperature till completion as was observed by TLC (TLC mobile phase: 25% methanol, 75% chloroform; Staining ninhydrin). The reaction was evaporated under reduced pressure to dryness by rotovapor followed by purification by a flash chromatography system. The product was eluted by a gradient of 23%-50% methanol in chloroform. The pure fractions were combined and evaporated to dryness. The solid obtained—173.5 mg was tested by TLC and used as is in the next step.

### Synthesis of DSPE-PEG1000-D-Aminosuccinic Acid (Aspartic Acid):

To 135.5 mg of crude DSPE-PEG1000-N-BOC-D-aspartic acid-1-tert-butyl ester (see step B), 2 ml of chloroform was added followed by the addition of 200 TFA. The reaction solution was stirred overnight at 4° C. till completion, as was observed by TLC (TLC mobile phase: 35% methanol, 65% chloroform; Staining ninhydrin). The reaction was evaporated under reduced pressure to dryness. To the obtained solid—152.5 mg 5 ml of diethyl ether were added gradually, and the reaction was stirred. Then the solution was centrifugated, and the liquid phase evaporated to dryness. The obtained product—72.3 mg was tested by TLC and H-NMR. The product was stored in a -80° C. freezer.

### Synthesis of DSPE-PEG1000-GlutarylChloride-Serotonin:

To 80 mg of DSPE-PEG1000 (0.0454 mmol), 80 ml of chloroform were added, followed by the addition of 2 ml triethylamine. The reaction was stirred, and 7.7 mg of glutaryl chloride (0.0454 mmol) was added gradually. The reaction was stirred for 4 hours until completion according to TLC (TLC mobile phase: 20% methanol, 80% chloroform; Staining ninhydrin). Then 3.76 mg (0.0177 mmol) of serotonin hydrochloride was added to crude DSPE-PEG1000-GlutarylChloride in 2 ml of DMF followed by the addition of 500 μl triethylamine, and all the reagents were stirred overnight at ambient temperature. The reaction solution was tested by TLC (TLC mobile phase: 69% chloroform, 26% chloroform, 5% water; Staining ninhydrin). The reaction was complete (there was an excess of serotonin hydrochloride and no reactant left after the reaction). The reaction solution was evaporated to dryness by rotovapor under reduced pressure, followed by purification by a flash chromatography system. The product was eluted by a 7-30% gradient of methanol in chloroform. The pure fractions were combined and evaporated to dryness. The obtained product—131 mg was confirmed by TLC. The product was kept in a −80° C. freezer.

### Synthesis of DSPE-PEG1000-GlutarylChloride-Memantine:

To 80 mg of DSPE-PEG1000 (0.0454 mmol), 80 ml of chloroform were added, followed by the addition of 2 ml triethylamine. The reaction was stirred, and 7.7 mg of glutaryl chloride (0.0454 mmol) was added gradually. The reaction was stirred for 4 hours until completion according to TLC (TLC mobile phase: 20% methanol, 80% chloroform; Staining ninhydrin). Then 11 mg (0.0614 mmol) memantine hydrochloride was added to crude DSPE-PEG1000-GlutarylChloride in 2 ml of DMF followed by 500 μl triethylamine, and all the reagents were stirred overnight at ambient temperature in an inert environment. The reaction solution was tested by TLC (TLC mobile phase: 20 methanol, 80% chloroform; Staining ninhydrin). The reaction was complete (there was an excess of memantine and no reactant left after the reaction). The reaction solution was evaporated to dryness by rotovapor under reduced pressure, followed by purification by a flash chromatography system. The product was eluted by a 20-40% gradient of methanol in chloroform. The pure fractions were combined and evaporated to dryness. The obtained product—44 mg was confirmed by TLC. The product was kept in −80° C. freezer. Synthesis of DSPE-PEG1000-Ritalinic acid:

### Synthesis of BOC-Ritalinic Acid:

To 500 mg of ritalinic acid (2.3 mmol) 50 ml of water were added, followed by the addition of 2 gr sodium carbonate anhydrous. Then 2.5 gr of di-tert-butyl decarbonate (11.0 mmol) was added gradually to the reaction solution that was stirred overnight at 50° C. in reflux. The reaction solution was evaporated by rotovapor under reduced pressure. The reaction was tested by TLC (TLC mobile phase: 10% methanol, 90% chloroform; Staining ninhydrin). 50 ml of 10% methanol, 90% chloroform were added to the reaction, which stirred for 20 min. Then the reaction was filtered and evaporated by rotovapor. The obtained solid—227 mg. For cleaning the product from free ritalinic acid, the obtained solid was purified by a flash chromatography system. The product was eluted with 10% methanol: and 90% chloroform. The pure fractions were combined and evaporated to dryness. The obtained product—is 96 mg. According to TLC, the purity was about 96%. Therefore, the product was used in the next step/steps. The product was kept at −20° C.

### Synthesis of BOC-Ritalinic Acid-NHS:

To 96 mg of crude BOC-ritalinic acid (0.3 mmol) 20 ml of 50% DMF, 50% chloroform were added followed by addition of 51 mg NHS (0.3 mmol). To the reaction solution, 87 mg of EDC (0.3 mmol) were added, and the reaction was stirred for 5 hours at ambient temperature before completion. The reaction was tested by TLC (TLC mobile phase: 9% methanol, 91% chloroform; Staining ninhydrin and exposure by UV light). For cleaning the product from free BOC-ritalinic acid and reagents, the reaction solution was diluted by a mixture of 80% hexane: and 20% chloroform. Then the solution was purified by a manual column. The product was eluted with 80% hexane: 20% chloroform. The pure fractions were combined. According to TLC, the purity was about 95%. Therefore the solution product (in DMF) was used in the next step/steps. The product was kept at −20° C.

### Synthesis of DSPE-PEG1000-Ritalinic Acid-BOC:

To 84 mg of DSPE-PEG1000 (0.0469 mmol), the solution product from the previous step (BOC-ritalinic acid-NHS) was added, followed by the addition of 200 μl triethylamine and 3 ml chloroform. The reaction was stirred for ON at ambient temperature before completion. The reaction was tested by TLC (TLC mobile phase: 10% methanol, 90% chloroform; Staining ninhydrin). Then the reaction was evaporated by rotovapor and filtered by a solution of 50% methanol and 50% chloroform. The obtained solid—134 mg.

### Synthesis of DSPE-PEG1000-Ritalinic Acid:

To 134 mg of crude DSPE-PEG1000-ritalinic acid-BOC, 2.3 ml of chloroform was added, followed by the addition of 200 μl TFA. The reaction solution was stirred for 2 hr. After 2 hr, the reaction was tested by TLC (TLC mobile phase: 10% methanol, 90% chloroform; Staining ninhydrin). According to the TLC, the reaction was completed, and by “dry loading method cleaning,” the final product was purified by flash chromatography. The obtained solid—143 mg. Then the product was purified by a flash chromatography system. The product was eluted with 15% methanol: 85% chloroform. The pure fractions were combined and evaporated to dryness. The obtained product—92 mg was confirmed by TLC. The product was kept at −80° C.

### Synthesis of DSPE-PEG1000-Cocaine:

### Synthesis of Benzoylecgonine:

To 200 mg of Cocaine hydrochloride (0.588 mmol), 9 ml of buffer solution (1.12 gr KH2PO4. 7.3gr K2HPO4) and Tetrahydrofuran (THF) (1 ml) were added and mixed for 3 hr at 80° C. The reaction was tested by TLC (TLC mobile phase: 80% chloroform: 20% methanol; Staining ninhydrin). Then the reaction was evaporated under reduced pressure to dryness by rotovapor. The product was clean by “soxlet extraction”. 162.2 mg pure product was obtained and confirmed by TLC (80% yield). The product was kept at 4° C.

### Synthesis of Benzoylecgonine-NHS:

To 51 mg of benzoylecgonine (0.167 mmol), 4 ml of dimethylformamide (2 ml), and chloroform (2 ml) were added, followed by the addition of 181 mg Sulfo-NHS (0.835 mmol). To the reaction solution, 320 mg of EDC (1.67 mmol) and one drop of 32% HCL were added, and the reaction was stirred for ON hours at ambient temperature before completion. The reaction was tested by TLC (TLC mobile phase: 55% chloroform: 45% methanol; Staining ninhydrin). The reaction was completed.

### Synthesis of DSPE-PEG1000-Cocaine:

Then, the reaction solution was filtered, and 50 μl of triethylamine and 100 mg DSPE-PEG1000 were added. It stirred for three hr. at ambient temperature. Then, it was tested by TLC (TLC mobile phase: 80% chloroform: 20% methanol; Staining ninhydrin). The reaction was completed. It was evaporated under reduced pressure to dryness by rotovapor and purified by a flash chromatography system. The product was eluted with 7% methanol: 91% chloroform. The pure product fractions were combined and evaporated to dryness. The obtained product—91.1 mg was identified by TLC. The product was kept at −80° C.

### Example 2: Small Molecules Targeted Nanoparticles

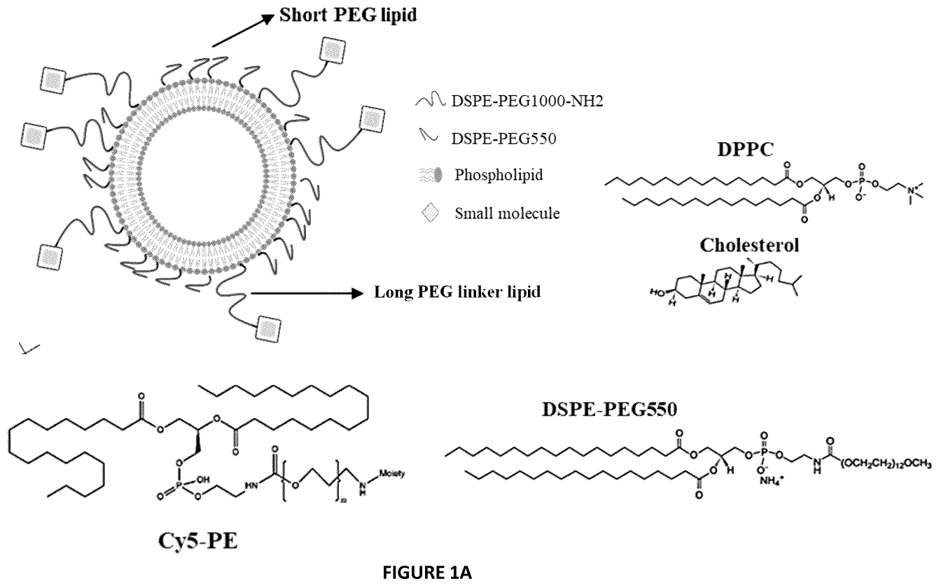

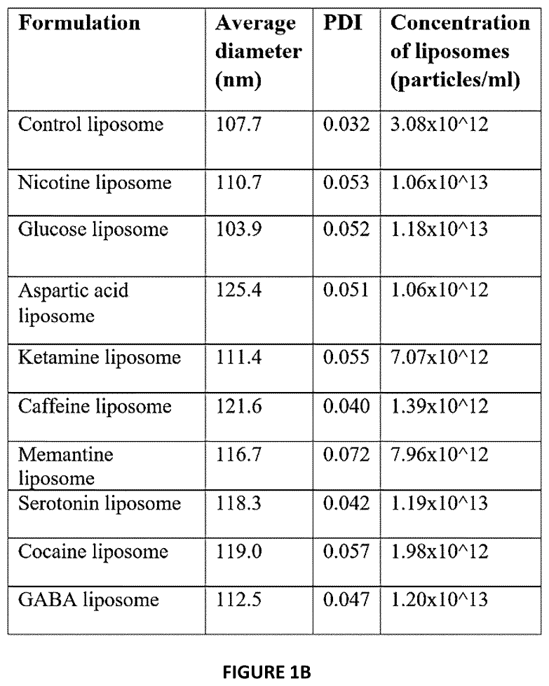

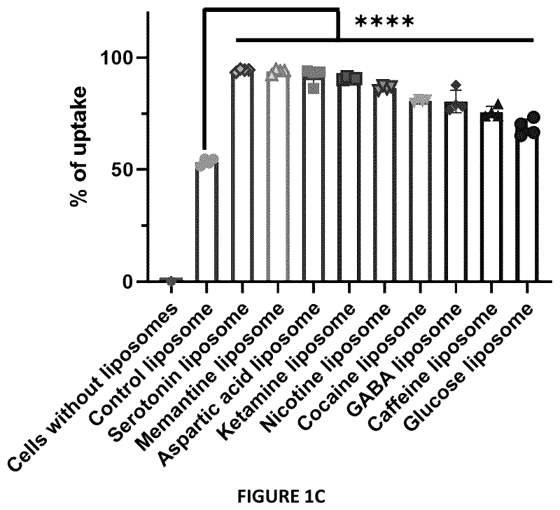

The inventors fabricated different liposomal formulations by the thin film method. The liposomes were composed of (16:0, DPPC), cholesterol, DSPE-PEG1000-small molecule moiety, DSPE-PEG-550, and DSPE-Cy5 (a fluorescent lipid for tracking) in the following molar percentages, respectively 64.4:30:2.5:2.5:0.6. (FIG. 1A). The average diameter (˜120 nm), particle concentration, and the PDI of each formulation were measured by Zetasizer Ultra (FIG. 1B.). The inventors tested the uptake kinetics of all the targeted liposomal formulations in the hCMEC/D3 cell line. The cells were incubated with 0.5 mM of targeted liposomes for 30 minutes and then were analyzed using flow cytometry. After 30 minutes, all the targeted liposomal formulations were taken in the highest quantity (˜2 fold) compared to the control formulation (without a targeting moiety) (FIG. 1C).

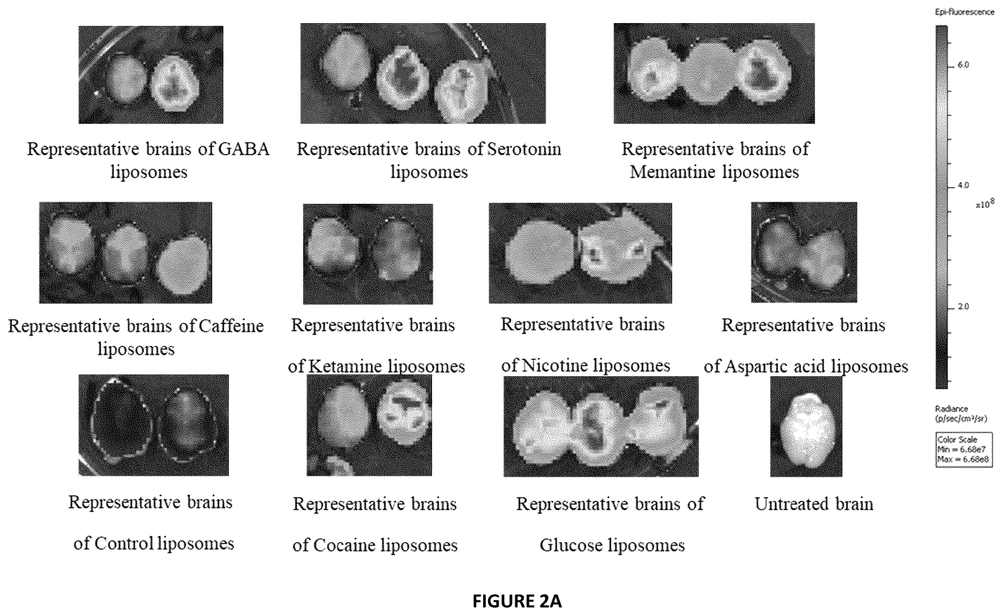

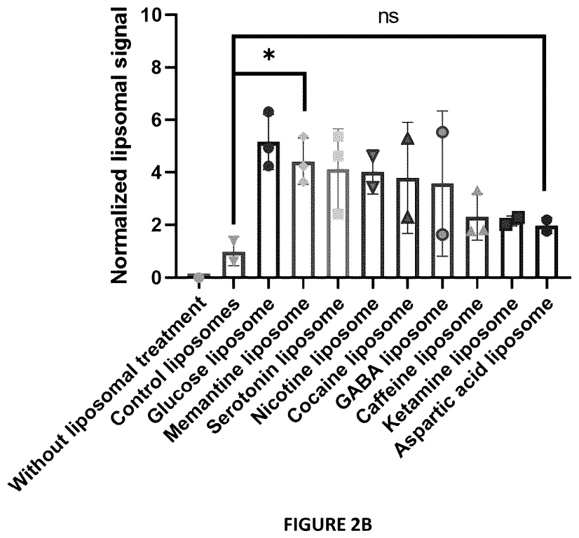

The targeted liposomes formulations and the control formulation (Cy5-labeled) were injected intravenously (150 μl) to healthy 8-10-week-old C57BL/6 black mice. 2 hours after injection, mice were perfused with PBS, and brains, spleens, lungs, and hearts were extracted and imaged by ex-vivo using the SpectrumCT Pre-Clinical In-Vivo Imaging System (IVIS) at an excitation of 640 nm and emission of 680 nm, binning 8, f-number: 2, and 0.5-seconds exposure (FIG. 2A). Quantitative data from all images were obtained using the ROI tool in LivingImage software. A control (non-injected) mouse was used for analysis, and its brain radiance was subtracted from the mean brain radiance of the injected mice. Then, the mean value of each mouse injected with the targeted liposome was normalized to the mean value of the mouse injected by control liposomes (FIG. 2B). All the targeted liposomal formulations demonstrated an upward trend in uptake compared to the control liposomes, with the strongest uptake observed for Glucose liposomes and Memantine liposomes (˜5-fold).

### Example 3: Encapsulation of Antibody in Active Targeting Nanoparticles

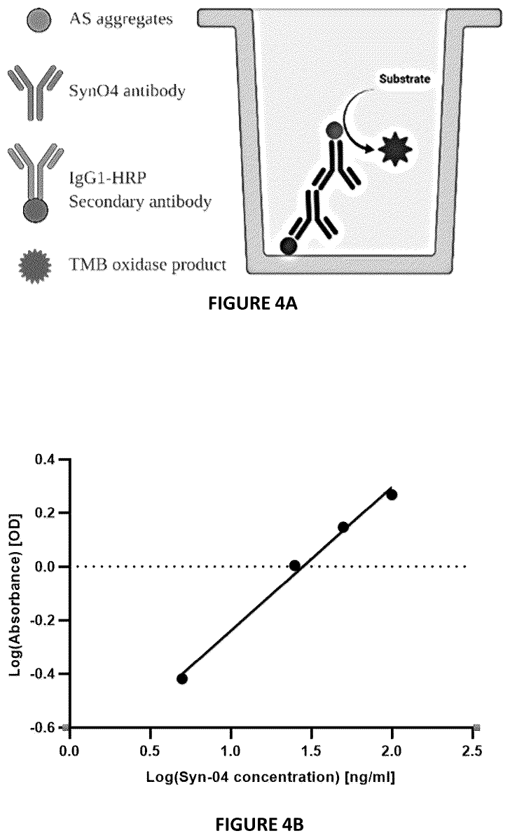

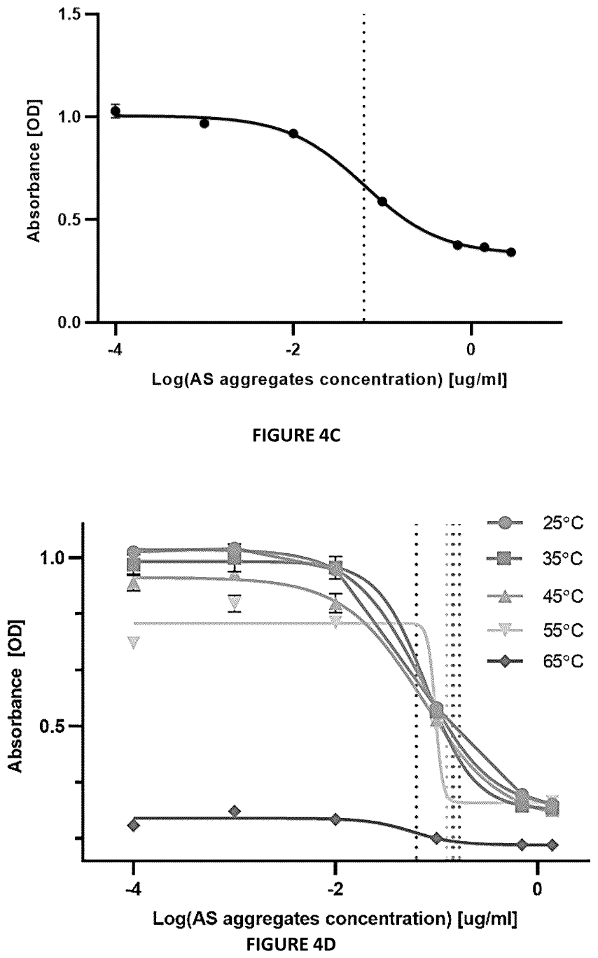

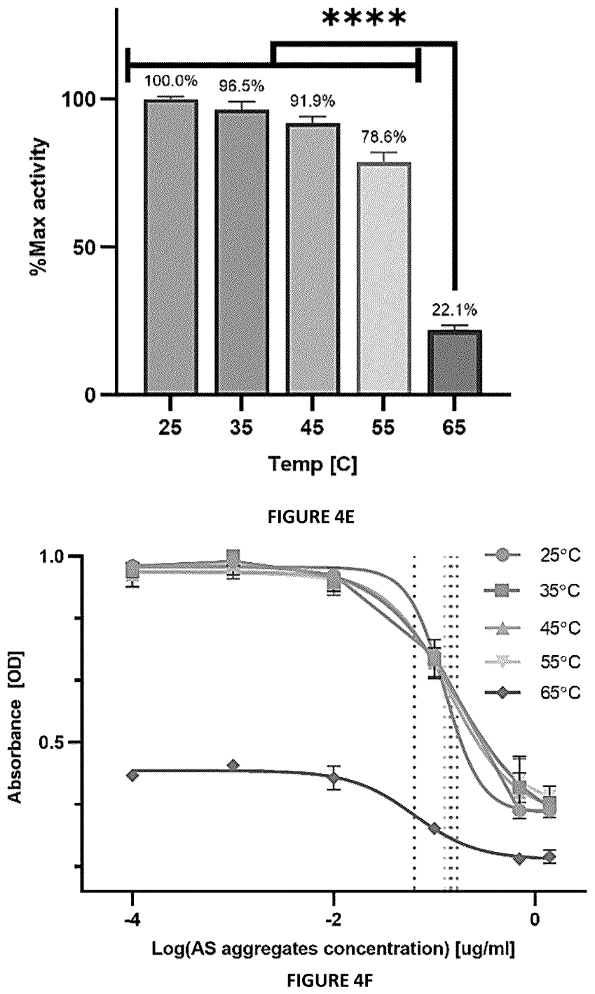

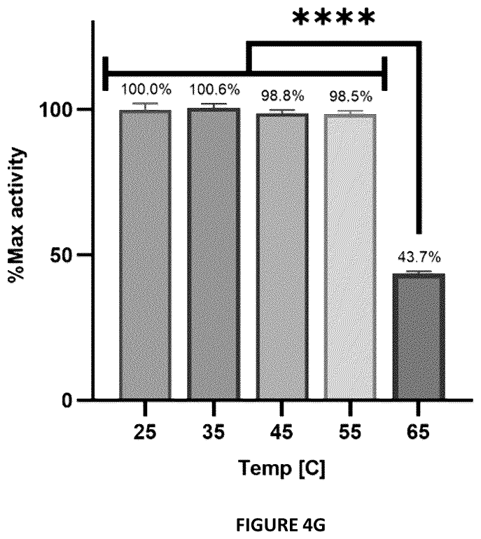

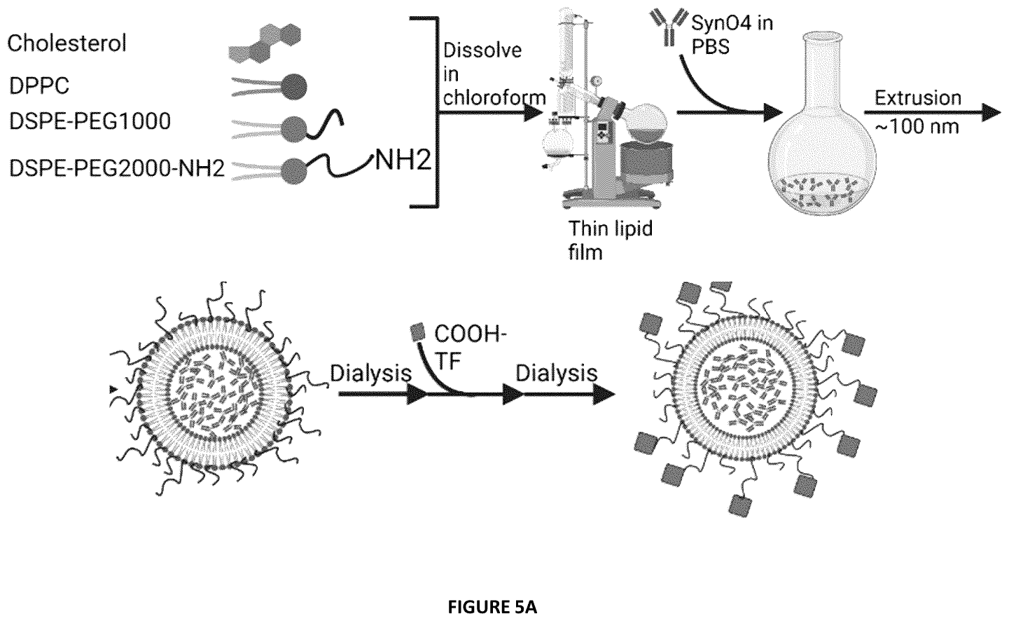

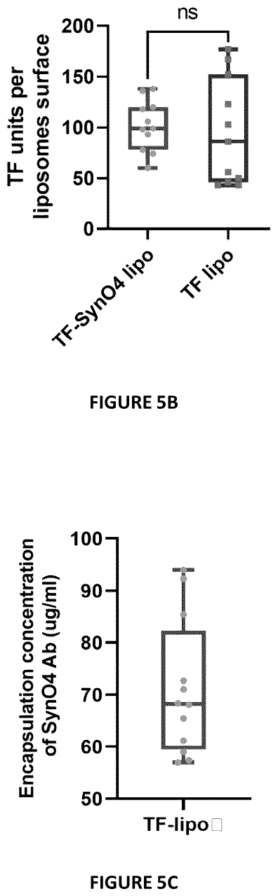

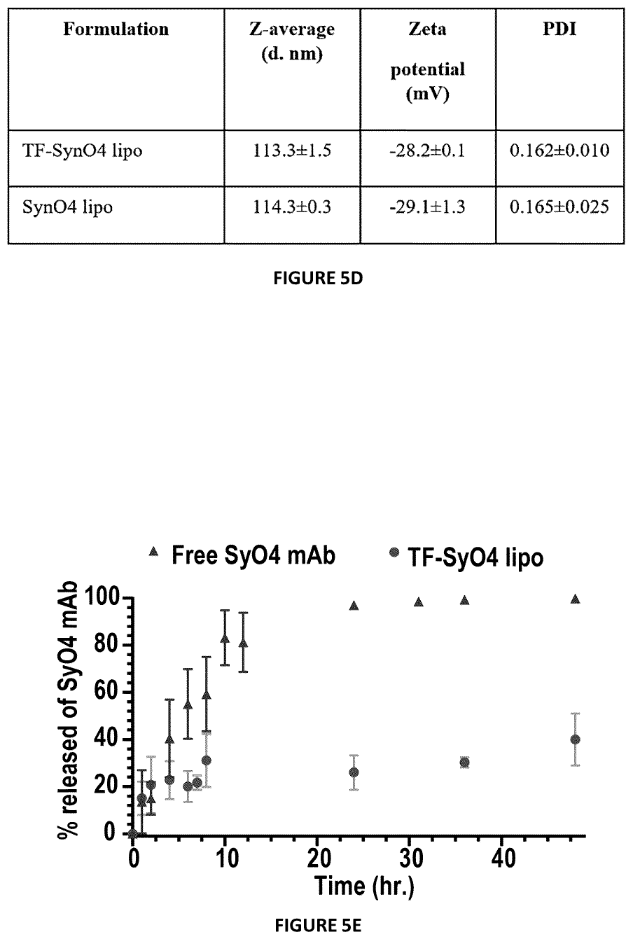

The inventors have successfully encapsulated the SynO4 antibody (Creative Biolabs, USA) inside liposomes and characterized multiple parameters of the developed formulation. In addition, the inventors developed an in-house ELISA assay to determine the optimal encapsulation temperature for choosing a stable lipid composition that enables high encapsulation efficiency (FIG. 4-5). Moreover, the inventors optimized the protein moiety conjugation protocol to promote the highest encapsulation efficiency and to preserve antibody activity inside liposomes (FIG. 4, 5-6).

SynO4 antibody is unique as it solely binds to the oligomeric form of AS and does not bind to monomeric AS. Therefore, it allows beneficial treatment by targeting AS aggregates directly, whereas the monomeric form remains unaffected. In addition, the SynO4 antibody is specific to human AS; its host species is a mouse. Consenqualy, it is not immunogenic when injected intravenously (IV) into mice.

### Targeting Protein—Nanoparticles Engineering:

To target the PD diseased regions in the brain, the inventors modified the active ligands (Insulin, Lactoferrin, and Transferrin) to the liposome surface for targeting specific sites in the BBB and facilitating site-specific delivery.

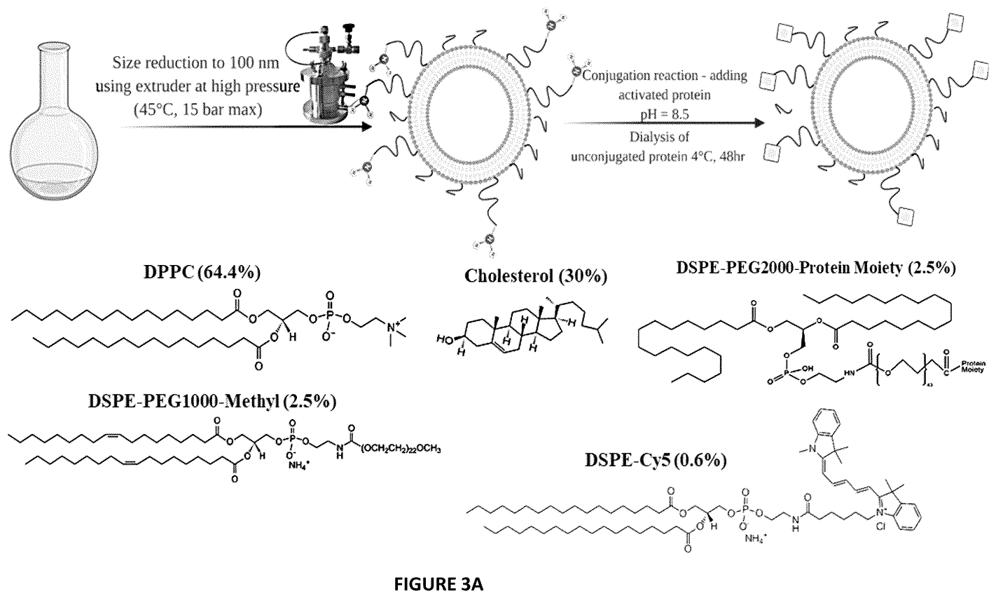

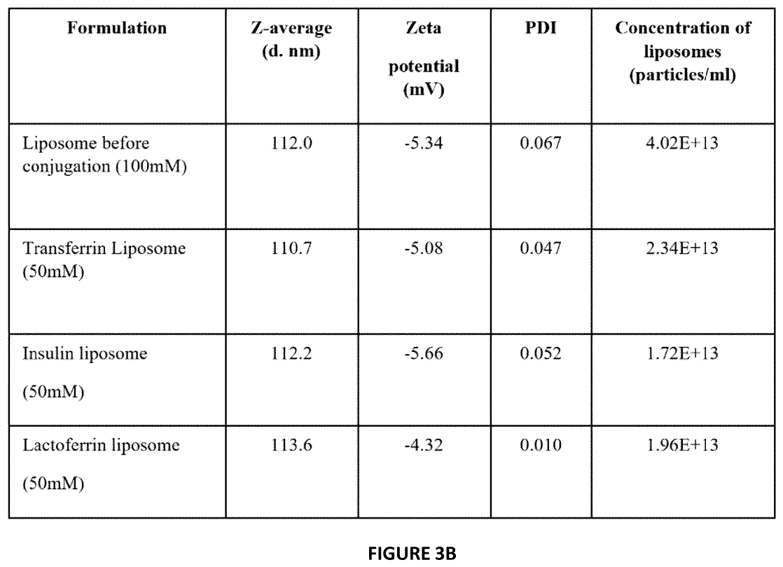

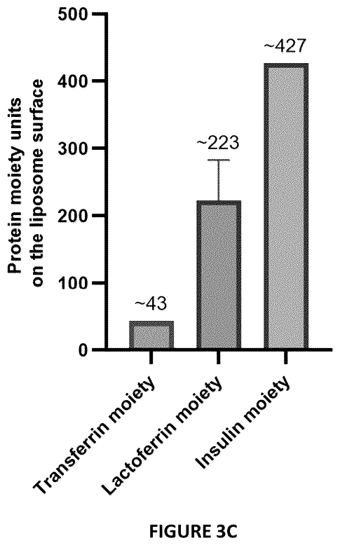

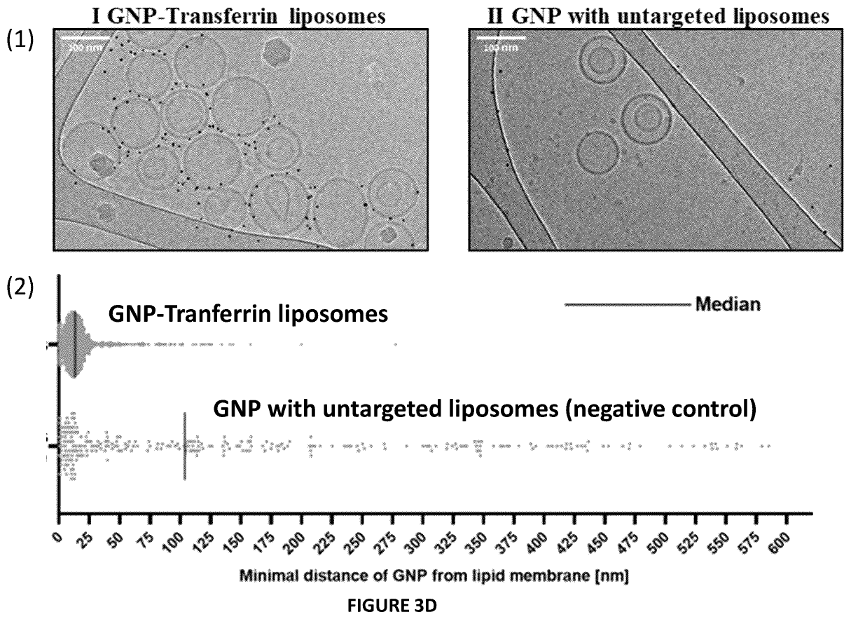

The main lipid that they chose to work with was DPPC. On the one hand, its Tm is below 45 degrees and, therefore, suitable for encapsulating biological drugs. On the other hand, DPPC allows the formation of a stable lipid nanoparticle formulation. The lipid composition was 64.4:30:2.5:2.5:0.6 of DPPC, cholesterol, DSPE-PEG2000-NH2, DSPE-PEG1000-Methyl, and DSPE-Cy5, respectively (FIG. 3A). All the liposomes were prepared using the thin film method and then extruded to reduce the size to 100 nm. The protein ligands were attached to the surface of the prepared liposomes by EDC carbodiimide crosslinking reaction. Their particle size, polydispersity (PDI), zeta potential, and particle concentration were determined by Zetasizer Ultra. (FIG. 3B). A BCA protein assay was used to quantify the protein-ligand conjugated concentration on the liposome surface (FIG. 3C). In addition, the inventors performed imaging by cryo-TEM. 5 nm Gold Nanoparticles (GNP) (10\hat{ }13 particles/ml conc.) were mixed with Transferrin-liposomes and untargeted liposomes (10\hat{ }13 particles/ml conc.) separately for 2 hr. Then, 10\hat{ }10 particles/ml from each sample were imaged by cryo-TEM. GNP-Transferrin liposomes (FIG. 3D 1(I)); GNP with untargeted liposomes (negative control) (FIG. 3D 1(II)). By Fiji imaging software analysis, a Scatterplot of GNP-liposomes was created. The median distance of a gold particle from a bilayer surface of Transferrin-liposome is about 15 nm compared to the negative control, which is about 100 nm, meaning there was no conjugation as expected (FIG. 3D (2)).

### Elisa Establishment:

To determine the working conditions with the SynO4 antibody, the inventors developed an in-house ELISA assay using the direct ELISA method (FIG. 4A). First, the 384-well plate was coated overnight with AS aggregates (Abcam). The next day, blocking was performed, followed by incubation with SynO4 antibody, washes, and secondary antibody (Goat polyclonal to mouse IgG1-HRP, Abcam, Ab6789). TMB substrate was used for signal development detected by a plate reader's absorbance measurement. For the generation of the IC50 curve, the SynO4 antibody was pre-incubated with a 10-fold serial dilution of AS aggregates. Based on the ELISA setup, the inventors generated a linear calibration curve for the SynO4 antibody (FIG. 4B) and a standard IC50 curve (FIG. 4C). Therefore, it confirms that chosen Elisa conditions work well and detect low antibody concentrations (above 0.7 ng/ml).

### Determination of Antibody Working Temperature:

The temperature at which the SynO4 antibody can withstand was tested. The antibody was pre-heated at different temperatures (35, 45, 55, and 65° C.) for 1 hr and followed by incubation with known concentrations of AS aggregates. After the incubation, the samples were measured by the ELISA to generate an IC50 curve. The IC50 curves for the 25° C., 35° C., and 45° C. temperatures were the same. A slight difference in the curve was noticeable for the 55° C. temperature, and a significant difference occurred in the curve for the 65° C. (FIG. 4D). A representation of the IC50 curves results by a percentage of Max activity; at 65° C., the maximal activity was reduced to 22% (FIG. 4E).

An additional activity experiment was conducted. However, after the incubation period, the samples were stored at 4° C., and the ELISA was measured only the next day. These conditions better simulate the process of liposome production because, during the liposomes' fabrication, there is a dialysis step at 4° C. for removing the unencapsulated drug. The IC50 curves for the 25° C., 35° C., 45° C., and 55° C. temperatures were the same. However, for the 65° C., a significant loss at the IC50 curve was evident (FIG. 4F). At 65° C., the maximal activity was reduced to 44%, which means that the antibody underwent recovery overnight at 4° C. (FIG. 4G). In addition, the experiment results indicate that the preferred allowed working temperature with the SynO4 antibody is 45° C. Due to this temperature threshold, three potential phospholipids were considered with a transition temperature (Tm) lower than 45° C.; POPC (16:0/18:1, Tm=−2° C.), DMPC (14:0, Tm=25° C.), and DPPC (16:0, Tm=41° C.). The Tm of the phospholipids is crucial as the unilamellar vesicles (i.e., liposomes) are only flexible enough to be downsized to 100 nm at a temperature above the Tm. Finally, the inventors chose to work with DPPC lipid because, in contrast to POPC and DMPC lipids, it ensures a high level of stability for the liposome structure.

### Characterization of TF-Targeted Lipid Nanoparticles Loaded With SynO4 Antibody:

The liposomes' synthesis is performed by following steps. First, the liposomes are fabricated by the thin film method. The lipids are dissolved in chloroform, and using a rotary evaporator; the chloroform is evaporated to form a thin homogenous lipid film (e.g., 100 mM). The film is hydrated with 2 mg/ml SynO4 antibody in PBS. Second, after the hydration, the liposomes are downsized to 100 nm using an extruder at 45° C. with a maximum working pressure of 15 bar. Finally, after removing the unencapsulated antibody by dialysis (48 hr.), using a 1000 kDa membrane against PBS, the protein-ligand is cross-linked to the surface of the liposomes by EDC and NHS reagents. After 2 hr. of reaction, the unreacted protein is removed by another dialysis (using a 1000 kDa membrane against PBS) for 48 hr (FIG. 5A). When the lipid in the formulation is DSPE-PEG2000-COOH, the reagents EDC, sulfo-NHS, and 2-mercaptoethanol (2-mercaptoethanol is added to quench the unreacted EDC) are added directly to the liposomes' solution. The 2-mercaptoethanol damaged the antibody inside the liposome, and thereby the activity of the antibody is lost. Probably due to the ability of 2-mercaptoethanol to cross the lipid membrane and reduce sulfur bonds in the antibody (data is not shown). Therefore, the inventors changed the conjugation steps using DSPE-PEG2000-NH2 instead of DSPE-PEG2000-COOH. This way, the reagents are added directly to the protein-ligand and removed before the addition of the activated protein to the liposome, preventing toxicity and damage to the encapsulated antibody.

The inventors fabricated two formulations—TF-liposome and TF liposome loaded with SynO4 mAb. TF-SynO4-liposome is composed of the following molar percentages of 64.4% DPPC, 30% cholesterol, 2.5% DSPE-PEG2000-(NH2)-TF, 2.5% DSPE-PEG-1000-Methyl and 0.6% DSPE-Cy5. TF-liposome is composed of the following molar percentages of 64.4% DPPC, 30% cholesterol, 2.5% DSPE-PEG2000-Methyl, 2.5% DSPE-PEG-1000-Methyl and 0.6% DSPE-Cy5. The Transferrin unit amount on the surface of the liposomes was quantified using a BCA assay (FIG. 5B). Approximately ˜100 units of TF moieties were detected on the lipid membrane in both formulations. The antibody's encapsulation concentration and activity were validated using an ELISA assay, demonstrating successful fabrication of targeted protein liposome encapsulating an active antibody (FIG. 5C). The physical parameters of particle size, PDI, and zeta potential were determined using Zeta-sizer Ultra (FIG. 5D). TF-SynO4 lipo and free SynO4 antibody were dialyzed separately against saline (1:10 volume ratio) at 37° C. at 200 rpm. The amount of free SynO4 in the intraliposomal solution was quantified at the desired time intervals by measuring 6 μl of a sample at the ELISA assay. The percentage of SynO4 released was determined by the ratio of free SynO4 released to total encapsulated SynO4 concentration (FIG. 5E).

### Example 4: In Vitro BBB Endothelial Cellular Uptake of Protein-Targeted Nanoparticles:

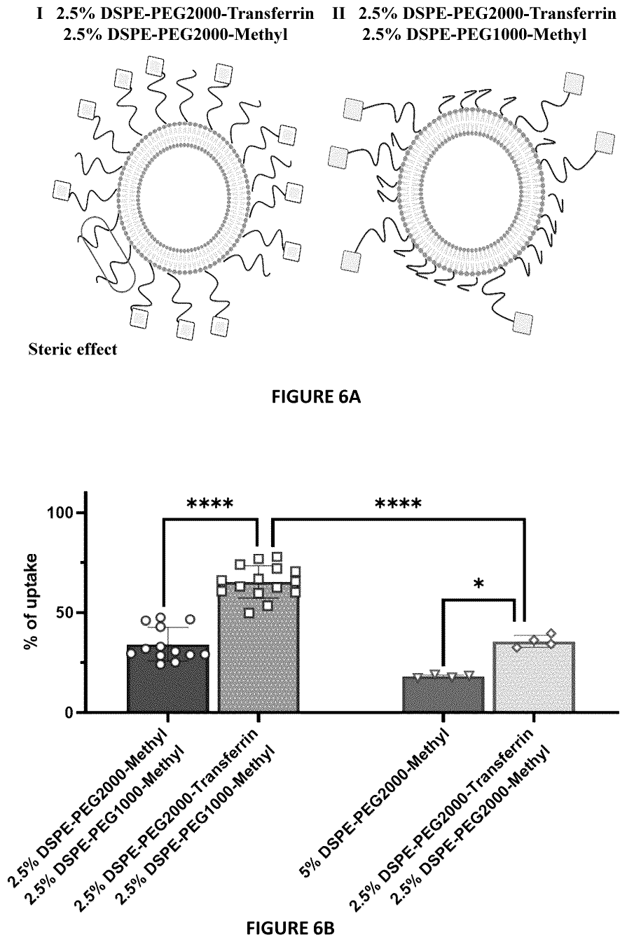

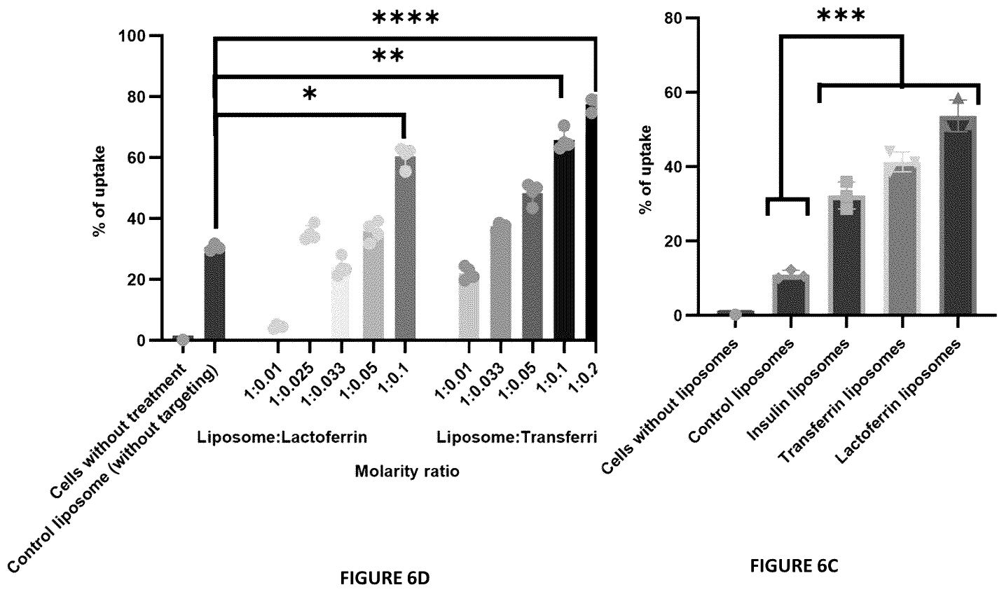

Furthermore, the inventors synthesized different liposomes formulations to test which composition formulation allows the best uptake efficiency in cell culture: I. DPPC (64.4%), cholesterol (30%), DSPE-PEG2000-NH2 (2.5%), DSPE-PEG2000-Methyl (2.5%) and DSPE-Cy5 (0.6%). II. DPPC (64.4%), cholesterol (30%), DSPE-PEG2000-NH2 (2.5%), DSPE-PEG1000-Methyl (2.5%) and DSPE-Cy5 (0.6%) (FIG. 6A). Another control formulations were prepared: DPPC (64.4%), cholesterol (30%), DSPE-PEG2000-Methyl (5%) and DSPE-Cy5 (0.6%); DPPC (64.4%), cholesterol (30%), DSPE-PEG2000-Methyl (2.5%), DSPE-PEG1000-Methyl (2.5%) and DSPE-Cy5 (0.6%). To formulation number I and formulation number II, Transferrin protein was added and conjugated by EDC crosslinking reaction. The liposomal formulations' uptake was tested in vitro in brain microvascular endothelial cell line—hCMEC/D3. The cells were incubated with 0.5 mM liposomes treatment for 30 minutes and then analyzed using flow cytometry. After 30 minutes, formulation number I was shown the highest uptake percentage compared to the other formulations (FIG. 6B). Using 2.5% of a long PEG linker and 2.5% of a short PEG lipid prevents the steric effect between the lipids' tails at formulation number II. Therefore, in all experiments, the ratio of a long PEG lipid linker to a short PEG lipid was maintained at 2:1.

Next, the inventors tested the uptake kinetics of Insulin liposomes, Lactoferrin liposomes, and, Transferrin liposomes compared to control liposomes (untargeted) in hCMEC/D3 cells. All the protein targeted formulations were composed of DPPC (64.4%), cholesterol (30%), DSPE-PEG2000-NH2 (2.5%), DSPE-PEG1000-Methyl (2.5%) and DSPE-Cy5 (0.6%). The three proteins: Insulin, Transferrin, and Lactoferrin, were conjugated to the surface of the liposomes in the same molar ratio. The control liposome was composed of DPPC (64.4%), cholesterol (30%), DSPE-PEG2000-Methyl (2.5%), DSPE-PEG1000-Methyl (2.5%) and DSPE-Cy5 (0.6%). The cells were incubated with 0.5 mM of each formulation for 30 minutes and then were analyzed using flow cytometry. After 30 min, the Lactoferrin-liposomes and Transferrin liposomes were taken in the highest quantity compared to the control formulation (FIG. 6C).

To test the amount of protein on the liposome surface needed for maximum uptake, the inventors conjugated both Transferrin and Lactoferrin proteins in different molar ratios. Similarly, the hCMEC/D3 were incubated with 0.5 mM of liposomes for 30 minutes and finally analyzed using flow cytometry. When the amount of protein on the liposome surface increases, more particles penetrate the cells (FIG. 6D).

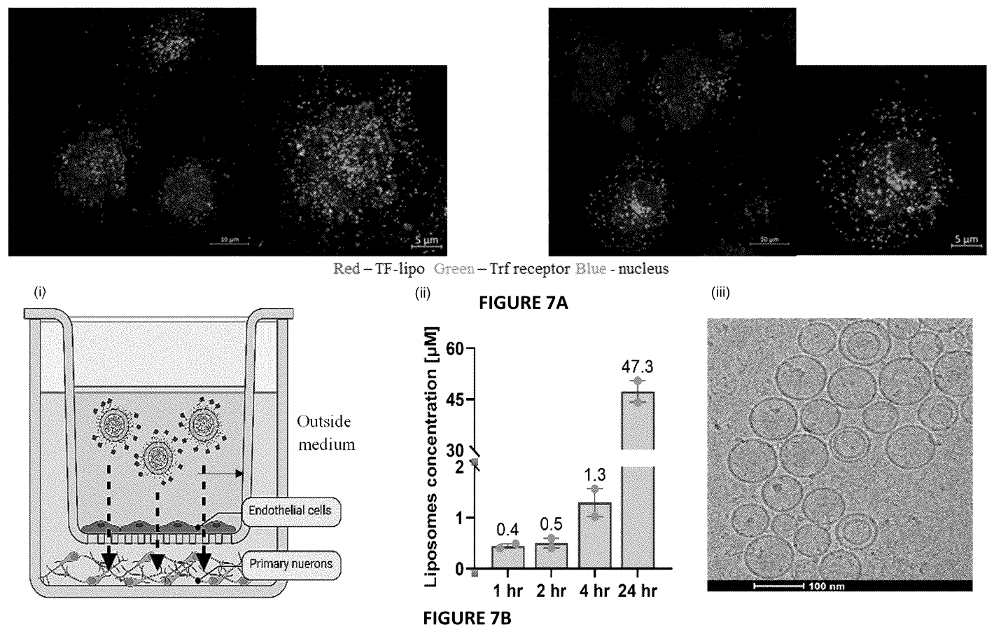

Brain endothelium is known to express many membrane receptors and transporters that specifically control the blood-to-brain transport of nutrients, including Insulin and Transferrin. Accordingly, hCMEC/D3 cells were tested for expression of transferrin receptors by super-resolution imaging. First, the cells were incubated with 2.5 mM of TF-liposomes labeled with Cy5 dye overnight. The following day, the cells were washed, fixed, and stained with anti-Trf-antibody (ab84036) and cell nuclei dye. Finally, they were imaged by super-resolution microscopy (FIG. 7A). The zoom-out and zoom-in images show the expression of Trfs and cellular uptake of TF-liposomes in the cell membrane and cytoplasm.

### Example 5: Crossing of TF-Liposomes in Cell-Based In Vitro BBB Model

The inventors investigated the ability of TF-liposomes to cross the BBB (FIG. 7B(i)). Specifically, the transport of TF-liposomes was evaluated across in vitro BBB model established in a transwell system made of BMECs in co-culture with primary cortical neurons (FIG. 7B(I)). TF-liposomes were labeled with Cy5 fluorescent dye, and 5 mM was added to the donor chamber. 50 μl of samples extracted from the acceptor chamber at 1.0, 2.0, 4.0, and 24.0 hr were analyzed by Plate-Reader (λex=633 nm, λem=685 nm, FIG. 7B(ii)). The 24 hr.-sample was kept for cryo-TEM imaging to further confirm the integrity of liposomal lipid bilayers after crossing the BBB (FIG. 7B(iii)). The liposome concentration (uM) is shown in (FIG. 7B(ii)). It was obtained that the liposomal permeability increases over 24 hours. The cryo-TEM image in (Figure B(iii)) shows that the liposomes crossed the BBB in the form of intact liposomes (˜100 nm).

### Example 6: TF-SynO4-Lipo Uptake and Therapeutic Efficacy in Neurons Cells

### PD In-Vitro Model Establishment:

To investigate PD pathology, the inventors created a variety of models. SH-SY5Y cell line and primary neuron cells are one of the most frequently used cellular models to study PD. Chronic exposure to neurotoxins or overexpression of different types of alpha-synuclein (AS) can mimic a PD phenotype. Alpha-synuclein may form multimers by self-assemblage, which irreversibly produces insoluble aggregates. In particular, the alpha-synuclein gene mutation A53T can form oligomers and aggregates more efficiently and faster than other types of alpha-synuclein. Preventing alpha-synuclein aggregation is one of the options to avoid the loss of dopaminergic neurons, and the inhibition of alpha-synuclein aggregation has become a valid therapeutic target in PD.

### TF-SynO4-Liposomes Uptake and SynO4 Drug Release in Neuron Cells:

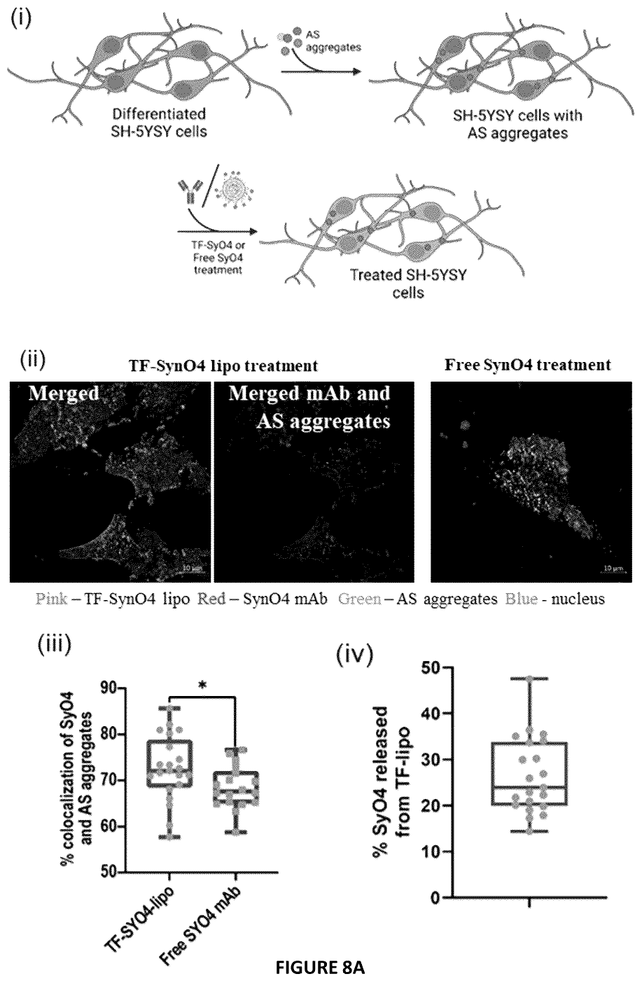

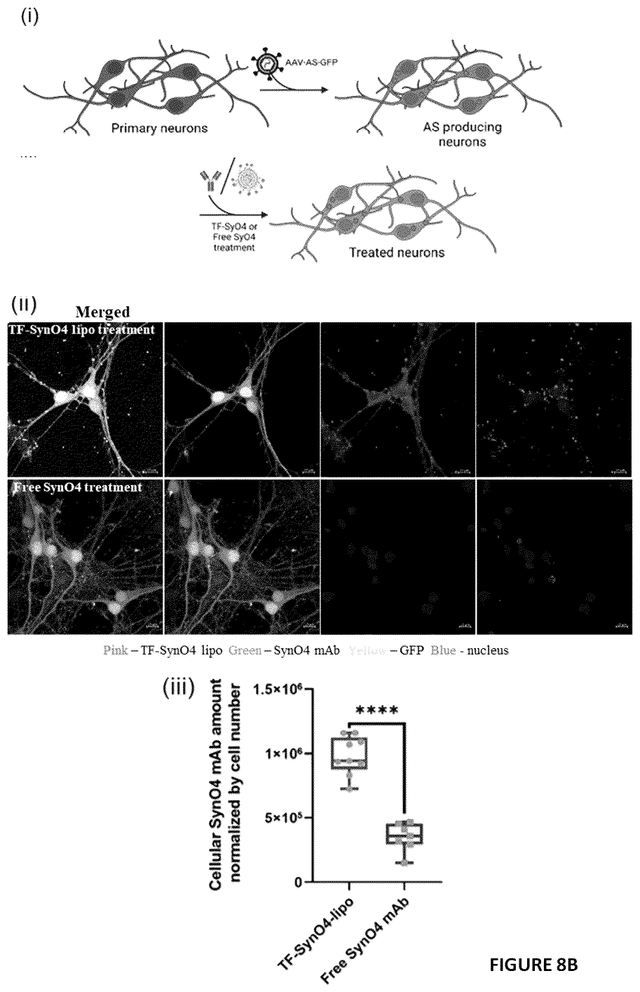

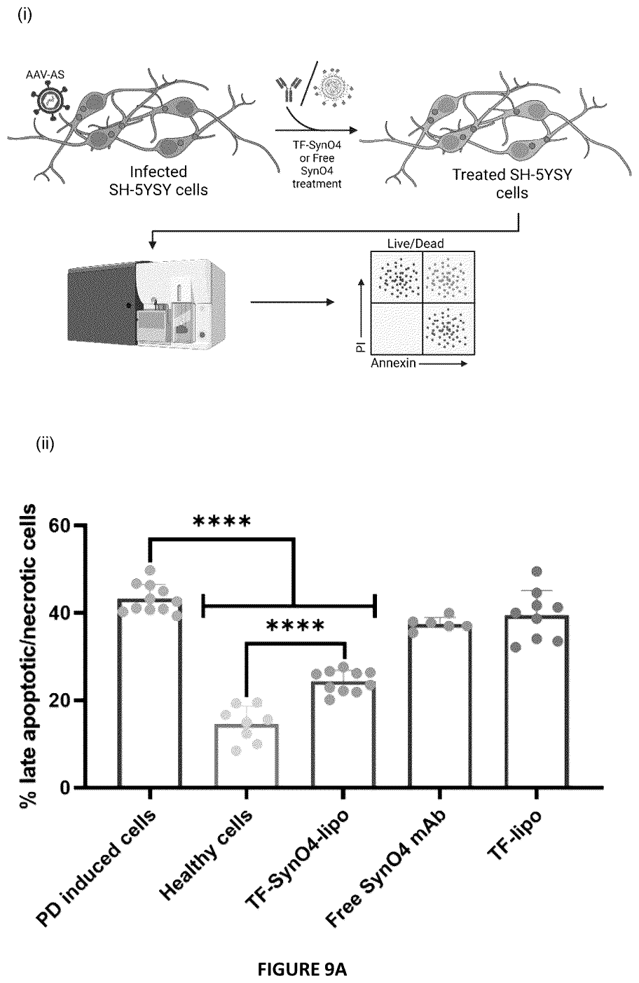

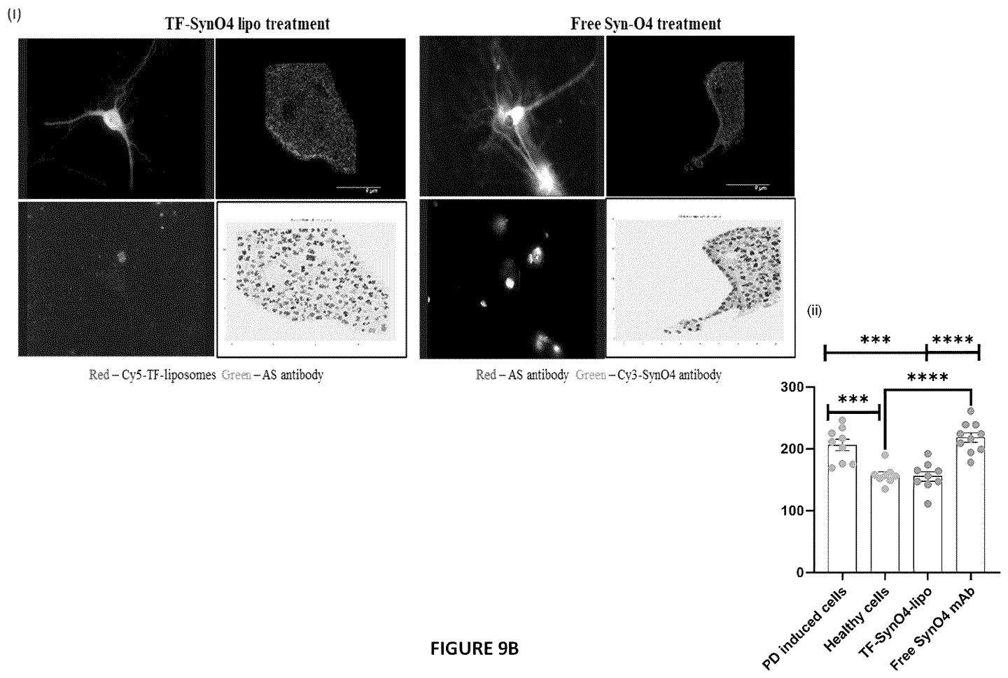

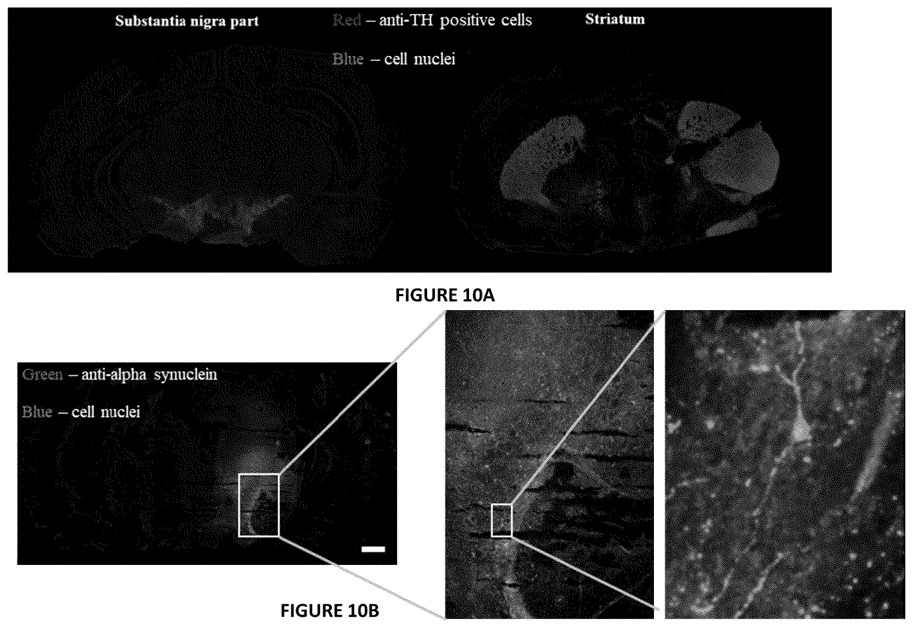

In FIG. 8, the inventors successfully showed a liposomal uptake in PD-differentiated SH-SY5Y cells, which was established by cell uptake of AS aggregates, and in PD-primary neurons, which was established by viral inducing (FIG. 8). In addition, the inventors demonstrated the release of encapsulated SynO4 antibody and its specificity for alpha-synuclein aggregates (FIG. 8A). Furthermore, in the PD-primary neurons, the inventors proved that the SynO4 concentration inside the cells is higher when the liposomes deliver the mAb than when it is free (FIG. 8B). Moreover, the inventors demonstrated a reduction in cell death and an inhibition of the accumulation of alpha-synuclein aggregates in cells by SynO4-liposomes (FIG. 9). On the other hand, free SynO4 mAb failed to reduce cell apoptosis and alpha-synuclein aggregation, correlating with its inability to penetrate cells, as shown in FIG. 8B.

The inventors established a PD model based on the uptake of recombinant human alpha-synuclein aggregates (ab218819) into differentiated SH-SY5Y (FIG. 8A). Cells were incubated with 0.1 uM Alexa-flour-labeled aggregates (Alexa Fluor® 488 Conjugation Kit, ab236553) for 6 hours, followed by extracellular alpha-synuclein aggregates removal and washes. Then, to test the liposomes' uptake and the antibody release, the cells were treated with TF-SynO4-liposomes (2.5 mM) or free SynO4 antibody (FIG. 8A(i)). Using super-resolution microscopy, the inventors demonstrated an uptake and presence of the labeled TF-SynO4 liposomes in the PD cells (FIG. 8A(ii)). The inventors also confirmed alpha-synuclein aggregates' uptake by the colocalization detected between the Cy3-SynO4 antibody and the Alexa Fluor 488-AS aggregates. The images reveal almost 75% colocalization of the SynO4 antibody (red) with the AS-aggregates (green) (FIG. 8A(iii)), demonstrating successful detection of the aggregated and antibody activity. The graph (FIG. 8A(iv)) shows that about ˜25% of antibody is released from the liposomes in the cells overnight.

In addition, the inventors investigated the uptake and release of the TF-SynO4-lipo (Cy5-labeled liposomes encapsulating Cy3-SynO4) in PD-primary neurons. Cells were infected using the pAAV-hSyn1-EGFP-(P2A)-a-Syn A53T-HA vector (the infection was done in Ori Ashery lab); to produce AS aggregation based on the overexpression of human A53T-AS (FIG. 8B(i)). When the human A53T-AS protein is expressed, the GFP protein in the AAV vector is expressed, resulting in the yellow color. After five days of infection, the cells were treated with the TF-SynO4-lipo or free SynO4 antibody treatments overnight (FIG. 8B(i)). Confocal images revealed the presence of liposomes (pink) in the PD-primary-neuron cells and the release of the SynO4 antibody in the cell cytoplasm (green) (FIG. 8B(ii)). No signal was found upon treatment with free Cy5-SynO4 antibody, demonstrating that free SynO4 antibody can't enter PD-primary-neuron cells (FIG. 8B(ii)). A graph (Figure B(iii)) shows that liposomes administer the antibody to cells at a higher level than free administration. The encapsulated SynO4 antibody was successfully delivered and preserved in both the uptake and release experiments (FIG. 8A, B).

### Testing of Therapeutic Effect of TF-SynO4-Lipo in PD-Differentiated SH-SY5Y Cells:

PD-differentiated SH-SY5Y cells were established by infection of AAV1/2-CMV/CBA-Human-A53Talpha-synuclein-WPRE-BGH-polyA (GD1001-RV, Charles River) vector (5.1×10\hat{ }11 VG/ml titer/concentration) to express mutant AS-aggregate. After 24 hours of infection, the cells were washed and incubated with TF-SynO4-lipo (0.05 mM), TF-lipo (0.05 mM) and free SynO4 (0.4214 ug/ml) overnight. Then, MEBCYTO Apoptosis Kit (Annexin V-FITC Kit) was used to detect the late-stage apoptotic/necrotic cells in the samples; the fluorescent signal of the samples was measured by flow cytometry (FIG. 9A(i)). The inventors demonstrate that treatment of TF-SynO4-lipo significantly reduces apoptotic cell levels compared to free SynO4 treatment. No significant effect was seen by TF-liposomes, meaning the therapeutic effect is due to the drug payload—the SynO4 mAb.

### Reduction of Alpha-Synuclein Aggregates Via Formulated TF-Liposomes With SynO4 Antibody in PD Primary Neuron Cells:

PD primary neuron cells infected by the pAAV-hSyn1-EGFP-(P2A)-α-Syn A53T-HA vector were treated with 2.5 mM Cy5-liposomes (SynO4) or free SynO4 parallel for 24 hours. The inventors quantified the alpha-synuclein aggregation level by using super-resolution with Bruker's Vutara 350 dSTORM microscopy and imaging analysis (FIG. 9). The fluorescence intensity of the cytoplasmic alpha-synuclein aggregates was calculated and analyzed for clusters using the Hierarchical Density-Based Spatial Clustering of Application with Noise (HDBSCAN) algorithm. Neurons treated with TF-SynO4-Liposomes have fewer alpha-synuclein aggregates than untreated neurons and neurons treated with free SynO4 mAb. Moreover, the number of alpha-synuclein aggregates didn't differ between the TF-SynO4-Liposomes condition and the healthy neurons. Therefore, it indicates that TF-SynO4-Liposomes treatment decrease alpha-Syn aggregation, while the free SynO4 mAb treatment does not (FIG. 9B (i, ii)).

### Example 5: Establishment of PD In-Vivo Model

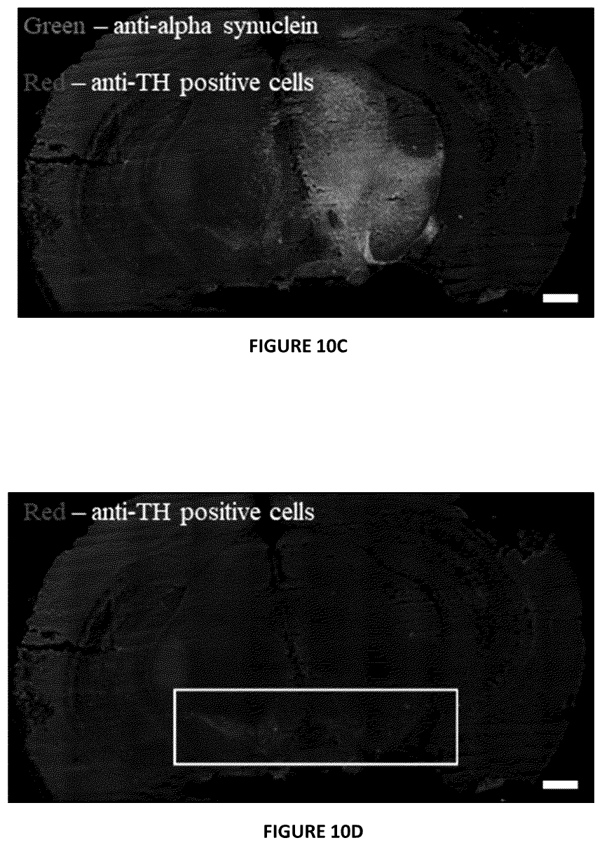

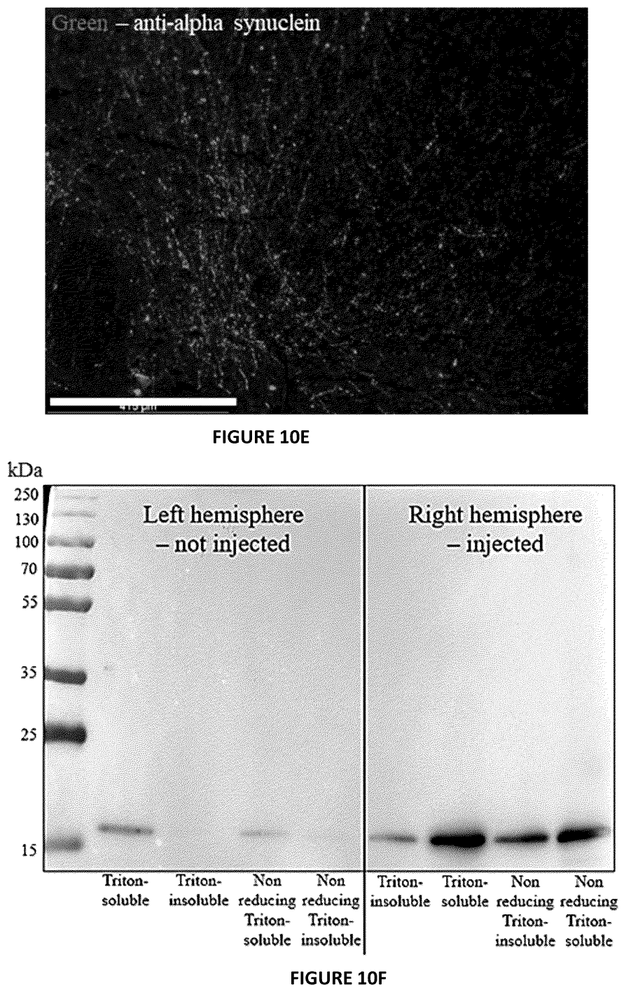

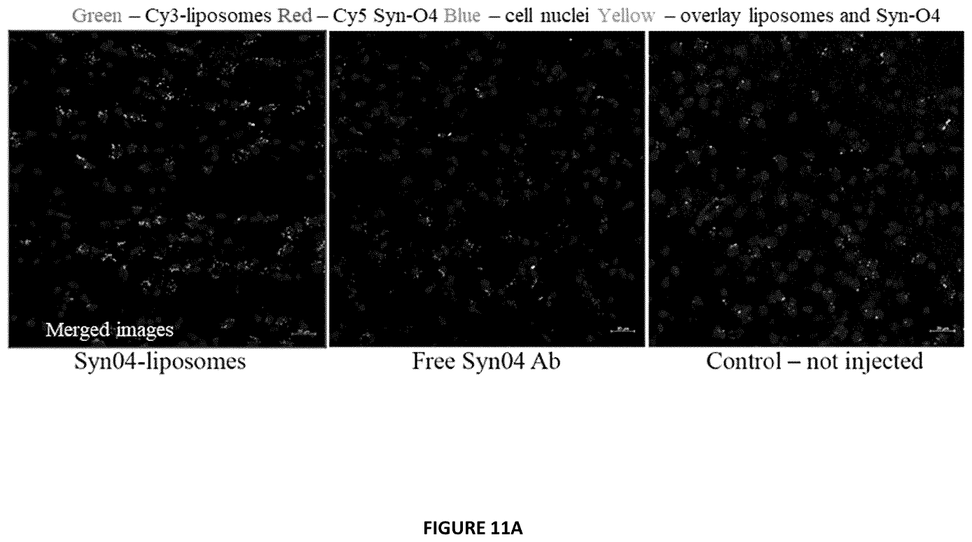

The inventors established and validated a viral-based PD in-vivo mice model by stereotactic injection of a viral vector expressing the human alpha-synuclein protein. The inventors demonstrated reduced TH positive cells, increased AS expression, and localization inside neurons (FIG. 10).

### Viral Model Establishment:

The viral vector was purchased from Sirion Biolabs in Germany, and its construct is—AAV2/6-hSyn1-Human SNCA-WPRE-polyA. Healthy 6-8 weeks c57BC/6JRccHsd male mice were injected with 1.5 μl of rAAV (1.63E13 GC/ml) directly to the right side substantia nigra (SN) using an automated stereotactic injection device equipped with the mouse brain atlas. Injected animals' well-being was monitored, and they did not express signs of motor dysfunction up to 4 weeks after injection. Injected animals were sacrificed 2 and 4 weeks after injection. The brain was collected after perfusion with PBS and immediately frozen in liquid nitrogen. Immunohistochemistry analysis:

The frozen brain was cut into 14 um slices using a cryostat (Leica) fixed with ice-cold acetone. Fixed sections were stained with rabbit monoclonal anti-human alpha-synuclein antibody (anti-AS, Abcam, Ab138501) and chicken polyclonal anti-tyrosine hydroxylase antibody (anti-TH, Abcam, Ab76442) as primary antibodies and goat anti-rabbit IgG alexa488 (Abcam, Ab150077) and goat anti-chicken IgY alexa555 (Abcam, Ab150174) as secondary antibodies, respectively.

In FIG. 10A, TH-positive neurons (red) are found in the SN and striatum of a healthy mouse brain. 2 weeks after injection, there is a clear expression of the human AS (green) in the right side of the mouse brain (FIG. 10B), the injected side, while the left side remains clean. Zoom-in images reveal the aggregation of the AS protein and its localization inside neurons (FIG. 10B). 4 weeks after injection, the spread of the AS expression is vaster (FIG. 10C), almost twice in size compared to the 2-week time point. Furthermore, the aggregates seem also to shift in the left hemisphere. In addition, there is a visible loss of TH-positive neurons in the right SN compared to the left (FIG. 10D), indicating the strong effect of AS aggregates on neuronal loss as expected. Finally, FIG. 10E shows a strong aggregation of AS 4 weeks after injection, with significant fibril-like structures in the SN area.

### Western Blot Analysis:

Western blot analysis demonstrates a robust alpha-synuclein expression in the right hemisphere compared to the left after two weeks (FIG. 10F). Frozen brain sections were divided into right and left hemispheres and homogenized in lysis buffer. First, initial homogenization was performed using a “gentle-MACS” device, followed by sonication on ice. Samples were centrifuged at 15,000×g for 1 hour at 4° C. The supernatant was termed “triton-soluble” fraction, and the pellet was re-suspended in lysis buffer with 2% SDS and termed “triton-insoluble”. Protein concentration was determined using the Bradford assay. 200 μg of protein extract was used to run an SDS-PAGE protein gel. Western blot was performed using an anti-human alpha-synuclein antibody (anti-AS, Abcam, Ab138501) and goat polyclonal anti-rabbit IgG-HRP (Abcam, Ab6721). It is important to note that the anti-AS antibody does not react with mouse alpha-synuclein, which means that the western blot results refer solely to the human alpha-synuclein originating from the viral expression.

### Example 5: Bioavailability In-Vivo Test

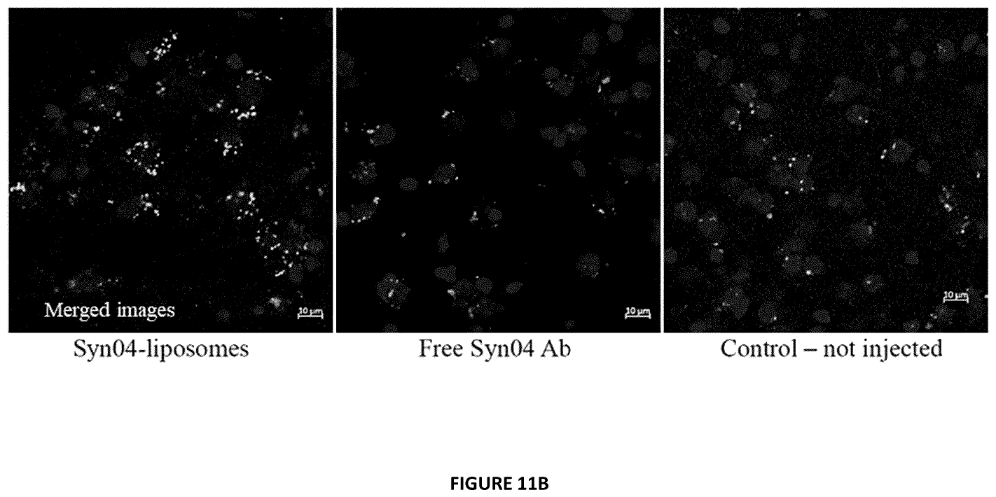

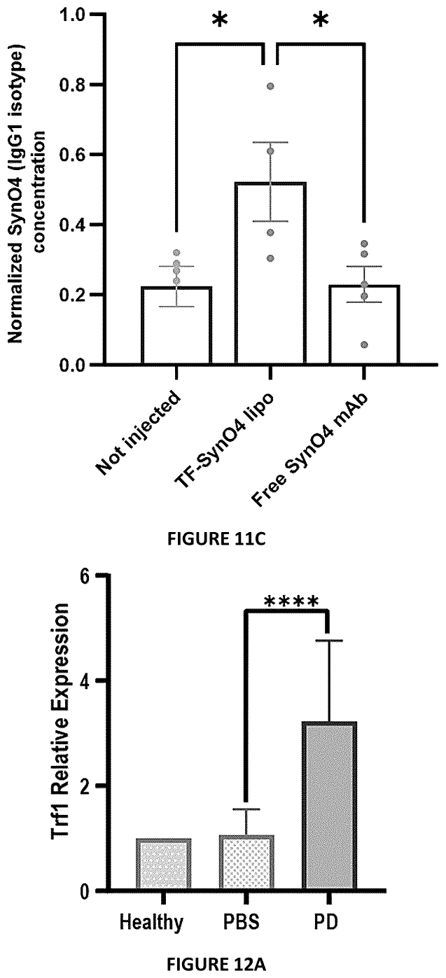

The inventors successfully delivered 1.2-fold more liposomal-originated SynO4 in PD-brain cells compared to the SynO4 antibody that was given by free (FIG. 11).

### Experiment Setup:

Twelve healthy 6-8 weeks c57BC/6JRccHsd male mice were injected with the PD-AAV. 4.5 weeks after viral induction, the PD mice were randomly separated into three treatment groups, n=4 in each group: 3 mg/kg of SynO4-liposomes, 7.5 mg/kg of free SynO4 antibody, and the control group (PD-induced brain without treatment). Mice were intravenously injected with the selected treatment and were sacrificed 24 hours post-injection. Mice were perfused with ice-cold PBS×1, and brains were frozen using liquid nitrogen and kept at −80° C. until further use.

### Fluorescent Histology Imaging:

16-μm frozen sections of fresh frozen brain tissue were cut using a cryostat machine. The frozen sections were kept at −80° C. until further use. Fresh frozen slices were mounted with DAPI-mounting and dried overnight. The sections were imaged the following day using confocal microscopy.

Images of the SynO4-liposomes injected brain show a clear uptake signal of the SynO4-liposomes (FIG. 11A, B). The yellow signal in the image represents the co-localization of liposomes labeled with Cy3 (green) and SynO4 antibody labeled with Cy5 (red). Brain images after injection of free SynO4 antibody show significantly less signal when compared to the control group (PD-induced brain without treatment). These results indicate that the delivery of SynO4-liposomes to the brain was successful compared to the free antibody.

### SynO4 Quantification in PD-Induced Brain:

Fresh frozen brain tissue was crushed into a fine powder using a pestle and mortar submerged in liquid nitrogen. The frozen powder was then transferred to RIPA buffer without SDS supplemented with phosphatase and protease inhibitors. After further homogenization, the lysate was centrifuged for 30 minutes at 12,000×g at 4° C. The supernatant containing the entire brain protein lysate was used for the experiment. Protein concentration was measured using the Bradford assay. Protein lysate was produced from the SynO4-liposomes, free SynO4, and no treatment groups, and 500 ug/ml of total protein from each group was used. The SynO4 antibody is a mouse-origin IgG1 isotype. Therefore, the detection was performed using the anti-mouse IgG1 Elisa assay kit according to the manufacturer's protocol (Abcam, ab133045). Results showed that after SynO4-liposomes injection, there were 1.2-fold (p<0.05, one-way ANOVA) more SynO4 antibodies detected in the brain compared to the brain injected with free SynO4 (FIG. 11C). In summary, the inventors delivered significantly higher antibody concentrations using liposomes than free antibody delivery.

### Example 6: Liposomes Brain Biodistribution In-Vivo Test

### RT-PCR of Trf1 Receptor Level in PD Brain:

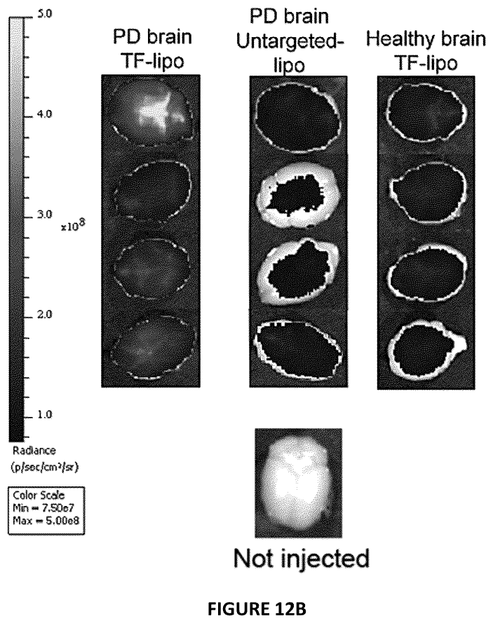

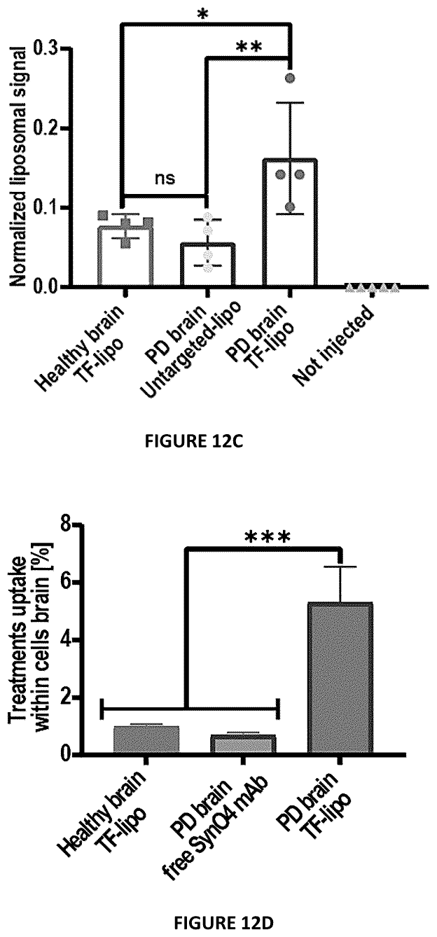

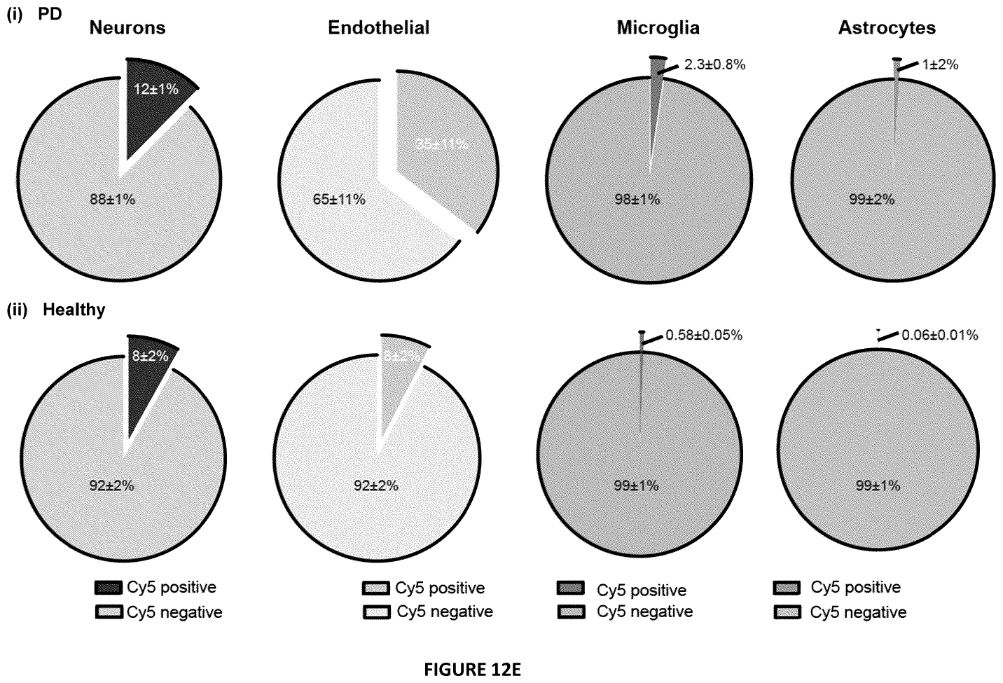

Five healthy 6-8 weeks c57BC/6JRccHsd male mice were injected with the PD-AAV, five 6-8 weeks c57BC/6JRccHsd male mice were used as a healthy control group, and five healthy 6-8 weeks c57BC/6JRccHsd male mice were injected with PBS. 8 weeks after viral induction/PBS, the mice were sacrificed and perfused with ice-cold PBS×1. The brains were frozen using liquid nitrogen and kept at −80° C. until further use. Outer sections of the BBB area of the brain were peeled off using a scalpel and transferred to −80° C. Frozen brain tissue was ground to a powder in liquid nitrogen using a mortar and pestle. Total RNA was extracted using an industrial NucleoSpin RNA Plus kit (Macherey-Nagel) following the manufacturer's instructions. Extracted RNA purity and quantity were evaluated using an Infinite 200PRO multimode reader (TECAN, Switzerland), while its integrity was assessed using gel electrophoresis (2% agarose, 35 minutes at 100V). Next, 400 ng of RNA was converted to cDNA using an industrial qScript cDNA synthesis kit (QuantaBio) following the manufacturer's protocol. Lastly, quantitative real-time PCR (qRT-PCR) was performed using qPCRBIO SyGreen Blue Mix Lo-ROX (PCRBIOSYSTEMS) with cycling conditions implemented according to manufacturer's instructions in QuantStudio1 (Applied Biosystems) real-time PCR thermal cycler. After that, transferrin receptor relative expression was calculated based on the 2^{-CCt} method. Before operating qRT-PCR, specific primers (transferrin forward: AAACACAGACGTGCTCCATCA (SEQ ID NO: 1) reverse: TCCTGCGTCCACTTTTGTCAT (SEQ ID NO: 2), and GAPDH forward: TGGGTGTGAACCACGAGAAA (SEQ ID NO: 3) reverse: GGGCCATCCACAGTCTTCTG (SEQ ID NO: 4)) were tested for their specificity by analyzing dissociation curves ranging from 60° C. to 95° C., optimal concentration, and amplification efficiencies using standard no template and no enzyme controls (FIG. 12A). The graph results indicate overexpression of Trf in PD-brain compared to the control groups.

TF-Lipo Vs. Untargeted-Lipo Brain Delivery:

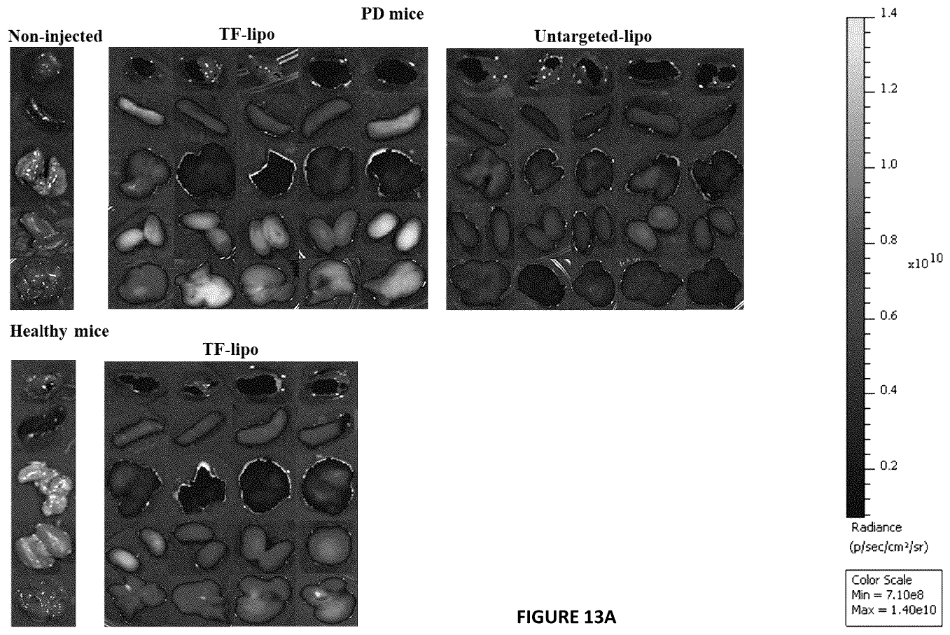

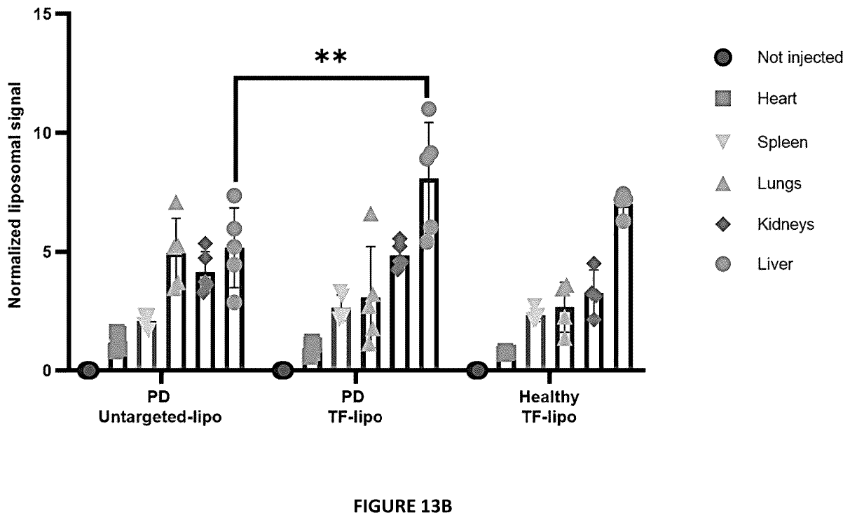

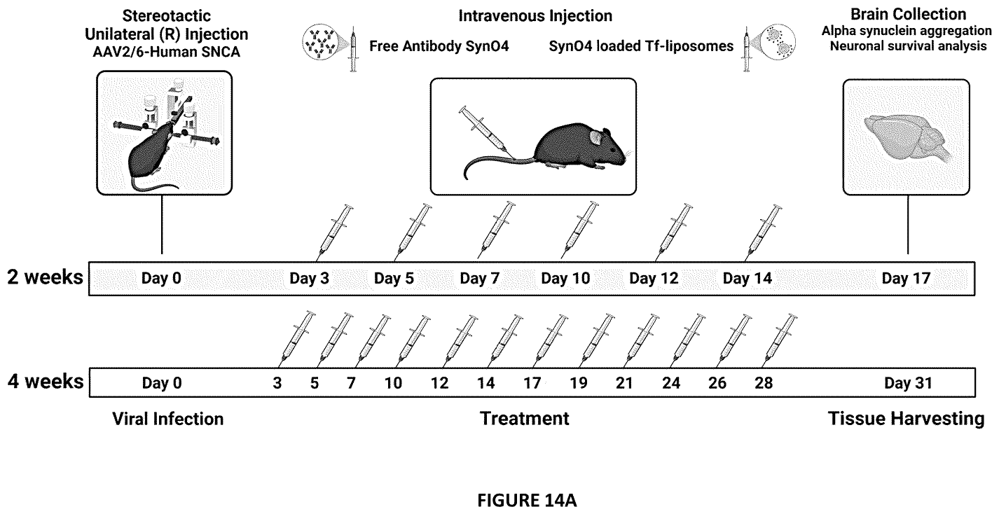

To further confirm the nanoparticles' targeting capacity, the liposomes' biodistribution along the organism was measured by ex vivo microscopy. The inventors explored the targeting capacity of liposomes to overcome the BBB and be accumulated in the brain by modifying the surface with transferrin. Liposome membranes dyed with Cy5 were used to track the biodistribution of targeted and untargeted carriers. The liposomes were intravenously injected into mice after eight weeks of alpha-synuclein viral injection, profiting from the increased expression of transferrin receptors in this situation (FIG. 12A). Twelve hours after administration, the brain, lungs, heart, liver, spleen, and kidneys were harvested. Liposomal biodistribution was assessed using ex vivo IVIS (In Vivo Imaging System) imaging (FIG. 12 B and C and FIG. 13). Quantitative data from all images were obtained using the ROI tool in Living Image software. A control (noninjected) mouse was used for analysis, and the average radiance of each tissue was subtracted from the average radiance of the injected mice. As shown in FIG. 12C, TF liposomes reach the brain and accumulate faster than untargeted liposomes. Since Trf1 receptors have been overexpressed in the BBB during PD, there is a significant increase in TF-lipo levels in PD brains compared to untargeted-lipo levels in healthy brains.

### Brain Cell-Level Liposomes Biodistribution:

Eight healthy 6-8 weeks c57BC/6JRccHsd male mice were injected with the PD-AAV, and four 6-8 weeks c57BC/6JRccHsd male mice were used as a healthy control group. Eight weeks after viral induction, the PD mice were randomly separated into two treatment groups, n=4 in each group: Cy5-TF-liposomes and free Cy5-SynO4 antibody (45.9 ug/ml). Mice were intravenously injected with the selected treatment and were sacrificed 24 hours post-injection. Mice were perfused with ice-cold PBSx1, and brains were homogenized into single cells using Adult Brain Dissociation Kit, mice, and rats (Cat. 130-107-677). To get clean samples of cells, meaning without debris and dead cells, a Dead Cell Removal kit (Cat. 130-090-101) was also used. Then, the cell samples (1×10\hat{ }6 cells/ml) were stained with a panel of antibodies: PE anti-mouse/human CD44 (Cat. BLG-103008), Brilliant Violet 711™ anti-mouse CD45 (Cat. BLG-103147), Brilliant Violet 421™ anti-mouse CD31 (Cat. BLG-102424), PE/Cyanine7 anti-mouse/human CD11b (Cat. BLG-101216), Anti-Mouse CD24 Antibody, Clone M1/69, Alexa Fluor® 488 (Cat. 60099AD,) and ACSA-2 Antibody, anti-mouse, APC-Vio770 REAfinity (Cat. 130-116-247). Finally, the samples were measured by flow cytometry (Cytek Aurora) (FIG. 12D). The inventors demonstrated that PD-brain cells took up significantly more liposomes than healthy brain cells. The difference in the percentage of cellular uptake between healthy and PD cells is due to the active targeting method that the inventors used and the BBB disruption in Parkinson's disease. In addition, the inventors show that more liposomes reach the brain and are taken up by PD cells compared to a free antibody. Specifically, the endothelial cells in both PD and healthy brains took up more liposomes than the other type of cells (FIG. 12E (i, ii)). This result is supported by the RT-PCR analysis graph (FIG. 12A). There is a significant overexpression of Trf in the PD-brain group. Therefore, endothelial cells known to express Trf took up more liposomes with transferrin on their surface. Additionally, the inventors demonstrate that the neuron cells also took a high percentage of liposomes—˜12% in the PD brain and ˜8% in the healthy brain (FIG. 12E (i, ii)).

### Liposomes Biodistribution in PD Mice Compared to Healthy Mice:

By ex-vivo imaging, the PD and healthy mice organs were imaged, and their fluorescence signal was quantified (FIG. 13). In the PD liver, TF liposomes accumulate significantly more than untargeted liposomes. The reason for this is that liver cells express transferrin receptors.

### Example 7: Testing the Therapeutic Effect of TF-SynO4-Lipo in PD In-Vivo Model

As previously discussed, one of the leading causes of Parkinson's disease is the aggregation, accumulation, and consequent toxicity of misfolded alpha-synuclein.

The inventors tested the effectiveness of TF-SynO4-lipo in reducing aggregation and toxicity. First, mice were unilaterally injected with viral vector (AAV2/6-hSyn1-Human SNCA-WPRE-polyA) encoding for human alpha-synuclein, increasing the protein production, and therefore be used as a PD in vivo model. Once the virus was injected, the mice were divided into 4 different groups, untreated (PD), free SynO4 monoclonal antibody (free Ab), transferrin targeted liposomes loaded with SynO4 antibody (liposomes), and healthy, where the AAV viral injection was not performed. As a comparison, we administered the free antibody at the same concentration as the liposomal formulation to assess the efficacy of the liposomes in delivering antibodies to the brain and neurons.

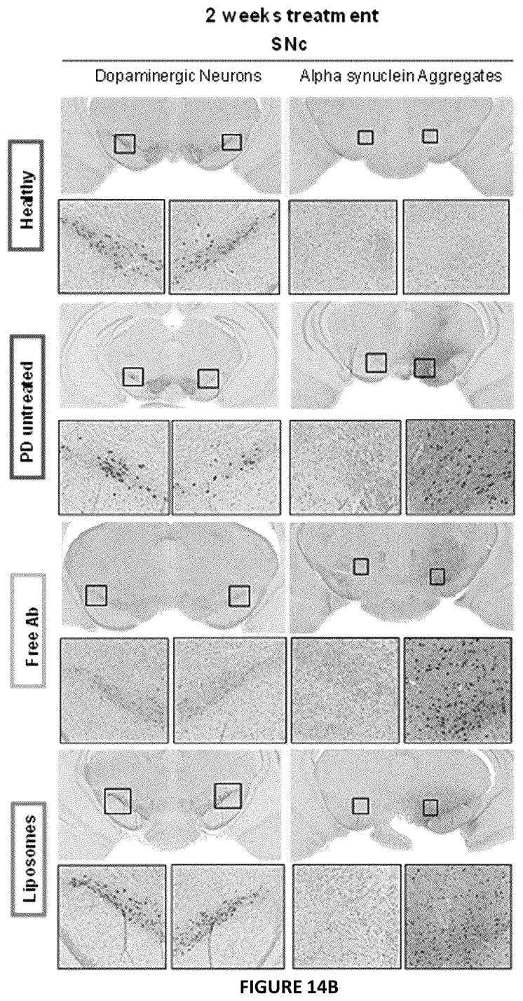

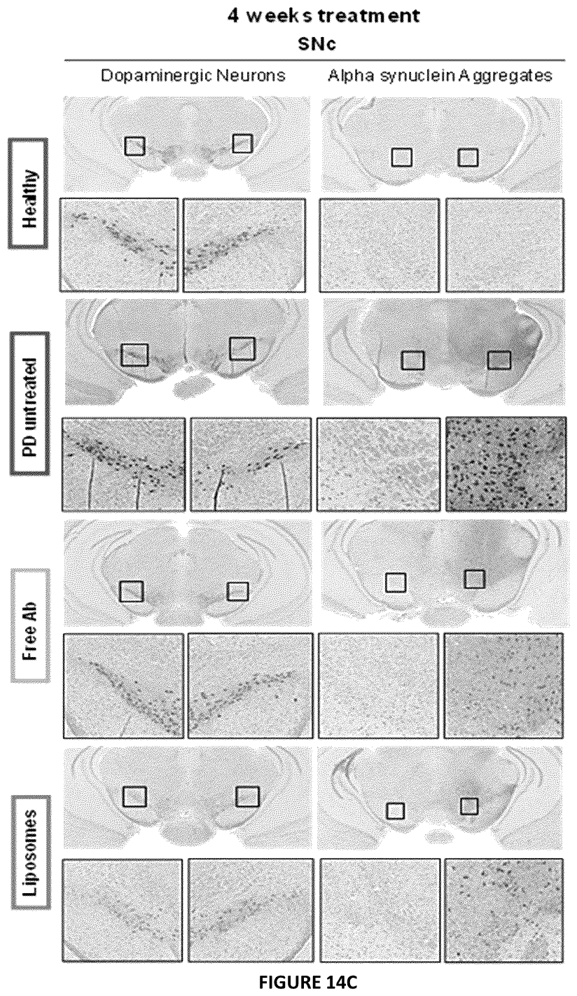

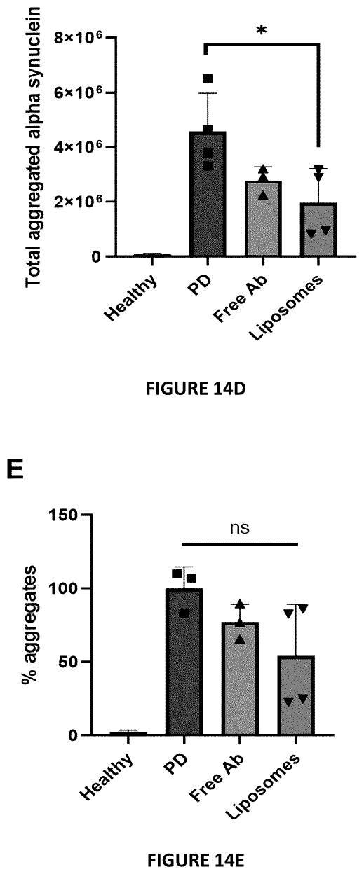

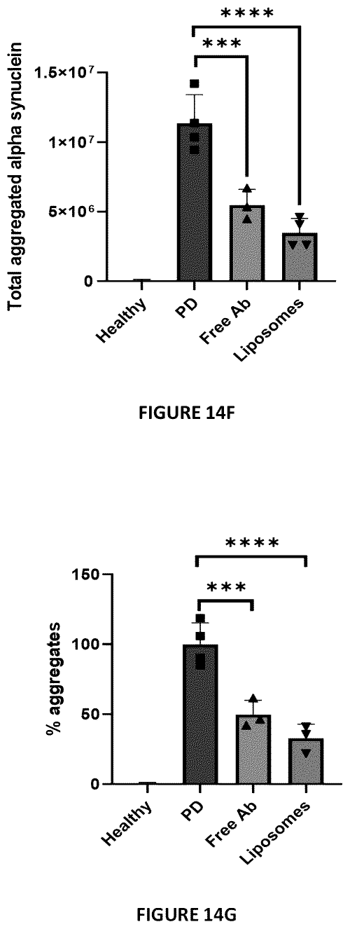

After three days post AAV injection, the different treatments were administered intravenously every other day for 2 and 4 weeks (FIG. 14A). After each time point, the mice were sacrificed, perfused, and the brain was harvested and fixed in PFA 4% for further histological analysis. Once fixed, the brain was embedded in paraffin and sectioned. Immunohistochemical analysis was performed in the substantia nigra (SN), where dopaminergic neurons are mainly located (FIGS. 14B and C). After two weeks of treatment, the total amount of aggregated alpha-synuclein in the SNc was significantly reduced by the liposomes' administration compared with the untreated mice (FIG. 14D). We evaluated the aggregated alpha-synuclein by immunohistochemistry, using the 5G4 clone antibody, which recognized aggregated and misfolded alpha-synuclein. FIG. 14D shows that the extra and intra cytoplasmic protein aggregation was reduced, achieving lower levels of aggregation in the case of the liposomes' administration compared with the free antibody treatment, whose difference compared with the untreated group was not significant.

On the other hand, analyzing only the extracytoplasmic aggregates found in the brain section, the differences between the different groups were insignificant (FIG. 14E). FIG. 4F shows that after 4 weeks of treatment, the disease progressed in the untreated group (PD) with a consequent increase in the total aggregated alpha synuclein compared with two weeks after AAV injection. In this case, the administration of the antibody delivered by liposomes significantly reduced the amount of total alpha synuclein (extra and intra cytoplasmic). On the contrary, the free antibody administration decreased the total aggregates, although its level of significance was lower than the one achieved by the liposomes' treatment. In terms of the number of extra cytoplasmic aggregates, the liposomal treatment reduced aggregation by 65%, as opposed to a 50% reduction with the free antibody.

Immunohistochemical and histological analysis revealed that TF-SynO4-lipo significantly reduced alpha-synuclein aggregation in PD mice.

Furthermore, the effect produced by the administration of our system was even higher than the effect generated by the administration of the free antibody. Consequentially, it proves the advantages of using a delivery system to overcome the different challenges in brain biological therapies, such as overcoming the BBB and penetrating neuron membranes. Therefore, our designed system represents a potential alternative to the current Parkinson's disease treatments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

1. A conjugate comprising a lipid covalently bound to a targeting moiety having a binding affinity to a CNS receptor, wherein:
the targeting moiety is bound to the lipid via a spacer; and
the targeting moiety has a molecular weight (MW) of less than 1000 Da.

2. The conjugate of claim 1, wherein the targeting moiety is a small molecule selected from the group consisting of Cotinine, GABA, Caffeine, Aspartic acid, Ritalinic acid, Ketamine, Serotonin, Memantine, and Cocaine, or any derivative, any isomer or metabolite thereof, and any combination thereof.

3. The conjugate of claim 1, wherein the spacer comprises a biocompatible polymer and wherein the spacer has a molecular weight (MW) between 1000 and 5000 Dalton (Da).

4. The conjugate of claim 1, wherein the biocompatible polymer is PEG.

5. (canceled)

6. The conjugate of claim 1, wherein the targeting moiety is bound to the spacer via an amide bond.

7. The conjugate of claim 1, wherein said lipid comprises a polar group comprising a primary amine.

8. The conjugate of claim 1, wherein the lipid comprises a phosphatidyl ethanolamine.

9. A nanoparticle comprising a core and a shell:
(i) the shell comprises a lipid layer, wherein the lipid layer comprises a phospholipid, a first modified lipid, an additional lipid and a sterol;
(ii) the core comprises a bioactive molecule,
wherein:
each of the first modified lipid and the additional lipid independently comprises a polymer covalently bound to a lipid;
the first modified lipid is covalently bound to a targeting moiety having a binding affinity to a CNS receptor; and wherein an average particle size of the nanoparticle is in the range between about 80 and about 150 nm.

10. The nanoparticle of claim 9, wherein the polymer comprises a biocompatible polymer: wherein MW of the polymer of the first modified lipid is between 1000 and 5000 Da; and MW of the polymer of the additional lipid is between 300 and 1000 Da.

11. The nanoparticle of claim 9, wherein the polymer is a polyether; optionally wherein the polyether is PEG; and wherein the phospholipid is characterized by a Tm of less than about 45° C.

12. The nanoparticle of claim 9, wherein the targeting moiety comprises any one of:
(i) A protein selected from Lactoferrin, Transferrin, and Insulin, or a combination thereof;
(ii) A small molecule selected from Cotinine, GABA, Caffeine, Aspartic acid, Ritalinic acid, Ketamine, Serotonin, Memantine, and Cocaine, or a combination of thereof.

13. (canceled)

14. The nanoparticle of claim 9, wherein a molar ratio between the sterol and the phospholipid is between 1:1 and 1:10.

15. The nanoparticle of claim 9, wherein a MW ratio between the polymer of the first modified lipid and the polymer of the additional lipid is about 2:1; wherein a molar ratio between the first modified lipid and the additional lipid is between 2:1 and 1:2; wherein a concentration of the first modified lipid within the nanoparticle is between 0.5 and 10% mol

16. (canceled)

17. (canceled)

18. (canceled)

19. The nanoparticle of claim 9, wherein said nanoparticle is a liposome.

20. The nanoparticle of claim 9, wherein the first modified lipid is the conjugate of claim 1.

21. A pharmaceutical composition, comprising the nanoparticle of claim 9 and a pharmaceutically acceptable carrier.

22. The pharmaceutical composition of claim 21 is formulated for systemic or local administration.

23. (canceled)

24. (canceled)

25. A method of preventing or treating a disease or disorder in said subject, the method comprising administering to said subject a therapeutically effective amount of the pharmaceutical composition of claim 21.

26. The method of claim 25, wherein the disease or disorder is a brain disease or disorder.

27. The method of claim 26, wherein said brain disease or disorder comprises a neurodegenerative disorder, a neuroinflammatory disorder, a proliferative disease, brain cancer, epilepsy, and other seizure disorders, mental disorders, stroke and Transient Ischemic Attack (TIA), and central nervous system (CNS) diseases, or any combination thereof.


SEQUENCE LISTING (4 sequences)

SEQ ID NO: 1 - DNA, 21 bp, synthetic construct
  source 1..21: mol_type = other DNA
aaacacagacgtgctccatca

SEQ ID NO: 2 - DNA, 21 bp, synthetic construct
  source 1..21: mol_type = other DNA
tcctgcgtccacttttgtcat

SEQ ID NO: 3 - DNA, 20 bp, synthetic construct
  source 1..20: mol_type = other DNA
tgggtgtgaaccacgagaaa

SEQ ID NO: 4 - DNA, 20 bp, synthetic construct
  source 1..20: mol_type = other DNA
gggccatccacagtcttctg